(19) United States
(12) Patent Application Publication
(10) Pub. No.: US 2018/0021315 A1
(43) Pub. Date: Jan. 25, 2018

(54) METHODS OF TREATING HEARING DISORDERS

(71) Applicants: Wayne State University, Detroit, MI (US); University of Arizona, Tucson, AZ (US); University of California Berkeley, Berkeley, CA (US)

(72) Inventors: Jinsheng Zhang, Troy, MI (US); Shaowen Bao, Tucson, AZ (US)

(21) Appl. No.: 15/655,222

(22) Filed: Jul. 20, 2017

Related U.S. Application Data
(60) Provisional application No. 62/364,580, filed on Jul. 20, 2016.

(51) Int. Cl. A61K 31/437; A61K 38/17

(52) U.S. Cl. CPC A61K 31/437; A61K 38/1793

(57) ABSTRACT

The present disclosure provides methods for treating a hearing disorder associated with maladaptive neuroplasticity, reduction of inhibition, shift of excitation-to-inhibition balance, changes in central gain, and/or changes in neural sensitivity in a subject by inhibiting the function and/or production of tumor necrosis factor alpha (TNF-α) in the subject. The present disclosure provides methods of administering to the subject a TNF-α inhibitory agent in an amount effective to treat the subject for a hearing disorder associated with maladaptive neuroplasticity, reduction of inhibition, shift of excitation-to-inhibition balance, changes in central gain, and/or changes in neural sensitivity. TNF-α inhibitory agents of the subject disclosure include agents that inhibit the function TNF-α, inhibit the production of TNF-α, inhibit TNF-α signaling, inhibit TNF-α expression, or inhibit TNF-α signaling pathway genes in the subject. The present disclosure also provides methods for treating a hearing disorder associated with maladaptive neuroplasticity, reduction of inhibition, shift of excitation-to-inhibition balance, changes in central gain, and/or changes in neural sensitivity in a subject by disrupting one or more alleles of a TNF-α signaling pathway gene in a cell of the subject.

### REFERENCE TO RELATED APPLICATION

This application claims priority from U.S. Provisional Patent Application Ser. No. 62/364,580, filed Jul. 20, 2016, the entire content of which is incorporated herein by reference.

### GOVERNMENT SUPPORT

This invention was made with government support under Grant No. W81XWH-15-1-0028 awarded by the Department of Defense and Grant No. DC009259 awarded by National Institute on Deafness and Other Communicative Disorders. The government has certain rights in the invention.

### FIELD OF THE INVENTION

Methods according to general aspects of the present invention relate to the inhibition of tumor necrosis factor alpha (TNF-α) for the treatment of hearing disorders, such as tinnitus, hyperacusis and auditory processing deficit/disorder, associated with maladaptive neuroplasticity, reduction of inhibition, shift of excitation-to-inhibition balance, changes in central gain, and/or changes in neural sensitivity in both auditory and non-auditory (such as limbic) systems. Methods according to specific aspects of the present disclosure relate to the inhibition of TNF-α for the treatment of hearing disorders associated with maladaptive neuroplasticity, reduction of inhibition, shift of excitation-to-inhibition balance, changes in central gain, and/or changes in neural sensitivity in human subjects.

### BACKGROUND OF THE INVENTION

Hearing disorders associated with maladaptive neuroplasticity, reduction of inhibition, shift of excitation-to-inhibition balance, changes in central gain, and/or changes in neural sensitivity can seriously impact affected individuals, at times leading to isolation and withdrawal. Studies have linked untreated hearing disorders to irritability, negativism, anger, fatigue, tension, stress, depression, avoidance of social situations, social rejection, loneliness, reduced job performance, reduced earning power, as well as diminished psychological and overall health.

There is a continuing need for treatments of hearing disorders associated with maladaptive neuroplasticity, reduction of inhibition, shift of excitation-to-inhibition balance, and/or changes in central gain and neural sensitivity.

### SUMMARY OF THE INVENTION

The present disclosure provides methods for treating a hearing disorder associated with maladaptive neuroplasticity, reduction of inhibition, shift of excitation-to-inhibition balance, changes in central gain, and/or changes in neural sensitivity in a subject by inhibiting the function and/or production of tumor necrosis factor alpha (TNF-α) in the subject. Aspects of the present disclosure include methods of administering to the subject a TNF-α inhibitory agent in an amount effective to treat the subject for a hearing disorder associated with maladaptive neuroplasticity, reduction of inhibition, shift of excitation-to-inhibition balance, and/or changes in central gain and neural sensitivity. TNF-α inhibitory agents of the disclosure include agents that inhibit the function of TNF-α, inhibit the production of TNF-α, inhibit TNF-α signaling, inhibit TNF-α expression, or inhibit TNF-α signaling pathway genes in the subject. The present disclosure also provides methods for treating a hearing disorder associated with maladaptive neuroplasticity, reduction of inhibition, shift of excitation-to-inhibition balance, changes in central gain, and/or changes in neural sensitivity in a subject by disrupting one or more alleles of a TNF-α signaling pathway gene in a cell of the subject.

One aspect of the present disclosure provides methods for treating a hearing disorder associated with maladaptive neuroplasticity, reduction of inhibition, shift of excitation-to-inhibition balance, changes in central gain, and/or changes in neural sensitivity in a subject by administering to the subject a TNF-α inhibitory agent that directly binds TNF-α, that directly binds a receptor for TNF-α, that inhibits the expression of TNF-α mRNA, that inhibits the translation of TNF-α protein, that inhibits the release of TNF-α from cells, or that inhibits downstream signaling from a receptor for TNF-α. Aspects of the present disclosure include methods for treating a hearing disorder associated with maladaptive neuroplasticity, reduction of inhibition, shift of excitation-to-inhibition balance, changes in central gain, and/or changes in neural sensitivity in a subject by administering to the subject a TNF-α inhibitory agent that is a small molecule, a polypeptide, or a nucleic acid.

Another aspect of the present disclosure provides methods for treating a hearing disorder associated with maladaptive neuroplasticity, reduction of inhibition, shift of excitation-to-inhibition balance, changes in central gain, and/or changes in neural sensitivity in a subject by administering to the subject a TNF-α inhibitory agent and providing the subject with a therapy in addition to the TNF-α inhibitory agent. Aspects of the present disclosure include methods for treating a hearing disorder associated with maladaptive neuroplasticity, reduction of inhibition, shift of excitation-to-inhibition balance, changes in central gain, and/or changes in neural sensitivity in a subject by administering to the subject a TNF-α inhibitory agent and a second agent useful in treating a hearing disorder associated with maladaptive neuroplasticity, reduction of inhibition, shift of excitation-to-inhibition balance, and/or changes in central gain and neural sensitivity.

Another aspect of the present disclosure provides methods for treating drug-induced tinnitus, injury induced tinnitus, blast induced tinnitus, noise-induced tinnitus, a hearing disorder associated with maladaptive neuroplasticity, reduction of inhibition, shift of excitation-to-inhibition balance, changes in central gain, and/or changes in neural sensitivity that is caused by injury (such as but not limited to noise and/or blast exposure), ototoxic drug/chemical agents (such as but not limited to aminoglycoside, gentamycin, cisplatin, carboplatin, salicylate, quinine), cochlear surgical insertions, aging, genetic factors, infections, autoimmune disease, and for treating conditions where the hearing disorder associated with maladaptive neuroplasticity, reduction of inhibition, shift of excitation-to-inhibition balance, changes in central gain, and/or changes in neural sensitivity is a primary condition, and conditions where the hearing disorder associated with maladaptive neuroplasticity, reduction of inhibition, shift of excitation-to-inhibition balance, changes in central gain, and/or changes in neural sensitivity is a secondary condition in the subject. Aspects of the present disclosure include methods for treating a hearing disorder associated with maladaptive neuroplasticity, reduction of inhibition, shift of excitation-to-inhibition balance, changes in central gain, and/or changes in neural sensitivity resulting from a traumatic brain injury (TBI).

Aspects of the present disclosure include methods for treating noise-induced tinnitus, blast-induced tinnitus, hyperacusis and auditory processing deficit.

Another aspect of the present disclosure provides methods for treating a hearing disorder associated with maladaptive neuroplasticity, reduction of inhibition, shift of excitation-to-inhibition balance, changes in central gain, and/or changes in neural sensitivity in a subject by disrupting one or more alleles of a TNF-α gene, a TNF-α receptor gene, or a downstream gene of the TNF-α signaling pathway in a cell of the subject.

Another aspect of the present disclosure provides methods for treating a hearing disorder associated with maladaptive neuroplasticity, reduction of inhibition, shift of excitation-to-inhibition balance, changes in central gain, and/or changes in neural sensitivity in a subject by administering a TNF-α inhibitory agent or disrupting one or more alleles of a TNF-α signaling pathway gene in a cell of the inner ear of the subject or a cell of the brain of the subject.

### BRIEF DESCRIPTION OF THE DRAWINGS

FIG. 1A is a graph showing impairment of gap detection performance at 7, 10, 14, and 20 kHz in wild type (WT) mice before, 2 days after, and 10 days after noise-induced hearing loss (NIHL);

FIG. 1B is a graph showing gap detection performance at 7, 10, 14, and 20 kHz in TNF-α knockout mice (KO) before, 2 days after, and 10 days after NIHL;

FIG. 1C is a graph showing prepulse inhibition performance at 7, 10, 14, and 20 kHz in WT mice before and 10 days after NIHL;

FIG. 1D is a graph showing prepulse inhibition performance at 7, 10, 14, and 20 kHz in KO mice before and 10 days after NIHL;

FIG. 1E is a graph showing auditory brainstem response (ABR) hearing thresholds at 4, 8, 16, and 32 kHz in WT mice after NIHL;

FIG. 1F is a graph showing ABR hearing thresholds at 4, 8, 16, and 32 kHz in KO mice after NIHL;

FIG. 2 shows contralateral (L) and ipsilateral (R) neuronal recoding maps for WT and KO in naïve and NIHL mice;

FIG. 3A is a graph showing the proportion of neurons responsive to ipsilateral and contralateral sound stimulation in naïve and NIHL WT and KO mice;

FIG. 3B is a graph showing the firing rate of neurons in response to ipsilateral and contralateral sound stimulation in naïve and NIHL WT and KO mice;

FIG. 3C is a graph showing the size of receptive fields measured in response to ipsilateral and contralateral sound stimulation in naïve and NIHL WT and KO mice;

FIG. 4A is a graph showing gap detection performance at 7, 10, 14, and 20 kHz in WT and KO mice before and after auditory cortical infusion of mouse recombinant TNF-α;

FIG. 4B is a graph showing gap detection performance at 7, 10, 14, and 20 kHz in WT and KO mice before and after auditory cortical infusion of albumin as a control;

FIG. 4C is a graph showing prepulse inhibition performance at 7, 10, 14, and 20 kHz in WT and KO mice before and after auditory cortical infusion of mouse recombinant TNF-α;

FIG. 4D is a graph showing prepulse inhibition performance at 7, 10, 14, and 20 kHz in WT and KO mice before and after auditory cortical infusion of albumin as a control;

FIG. 5A is a graph showing gap detection performance at 7, 10, 14, and 20 kHz in WT mice before and after systemic salicylate injection;

FIG. 5B is a graph showing gap detection performance at 7, 10, 14, and 20 kHz in KO mice before and after systemic salicylate injection;

FIG. 5C is a graph showing prepulse inhibition performance at 7, 10, 14, and 20 kHz in WT mice before and after systemic salicylate injection;

FIG. 5D is a graph showing prepulse inhibition performance in KO mice before and after systemic salicylate injection;

FIG. 6A is a graph showing the number of activated astrocytes in rat dorsal cochlear nucleus (DCN), inferior colliculus (IC), and auditory cortex (AC) combined for sham-blast-exposed rats, rats 1 day after blast exposure, rats 1 week after blast exposure, and rats 1 month after blast exposure;

FIG. 6B is a graph showing the number of activated astrocytes in rat DCN for sham-blast-exposed rats, rats 1 day after blast exposure, rats 1 week after blast exposure, and rats 1 month after blast exposure;

FIG. 6C is a graph showing the number of activated astrocytes in rat IC for sham-blast-exposed rats, rats 1 day after blast exposure, rats 1 week after blast exposure, and rats 1 month after blast exposure;

FIG. 6D is a graph showing the number of activated astrocytes in rat AC for sham-blast-exposed rats, rats 1 day after blast exposure, rats 1 week after blast exposure, and rats 1 month after blast exposure;

FIG. 7A is a graph showing gap detection results indicating gap-induced suppression of the startle response (grey bars) relative to the startle-only response (black bars) in a control tinnitus(−) rat at 8, 12, 16, 20, and 28 kHz and with broadband noise (BBN);

FIG. 7B is a graph showing gap detection results indicating a lack of gap-induced suppression of the startle response (grey bars) relative to the startle-only response (black bars) in a blast-exposed tinnitus(+) rat at 8, 12, 16, 20, and 28 kHz and with BBN;

FIG. 7C is a graph showing the relative number of entries and time spent in the open arms of an elevated plus maze for sham-blast-exposed rats and blast exposed rats;

FIG. 8A is a graph showing gap detection results for a pre-blast rat at 8, 12, 16, 20, and 28 kHz and with BBN;

FIG. 8B is a graph showing gap detection results for a post-blast, untreated rat at 8, 12, 16, 20, and 28 kHz and with BBN;

FIG. 8C is a graph showing gap detection results for a post-blast, thalidomide-treated rat at 8, 12, 16, 20, and 28 kHz and with BBN;

FIG. 9 is a graph showing the gap detection performance at 7, 10, 14, 20, and 28 kHz for mice pre-NIHL, post-NIHL, and mice receiving daily intraperitoneal thalidomide injections for three days post-NIHL;

FIG. 10 is a graph depicting blast-induced TNF-α protein expression in rat AC for control rats, vehicle-treated rats, and rats receiving 3,6′-dithiothalidomide;

FIG. 11 is a graph depicting blast-induced TNF-α protein expression in rat DCN for control rats, vehicle-treated rats, and rats receiving 3,6′-dithiothalidomide;

FIG. 12 is a graph depicting blast-induced TNF-α protein expression in rat IC for control rats, vehicle-treated rats, and rats receiving 3,6′-dithiothalidomide;

FIG. 13 is an image of a Western blot of blast-induced TNF-α protein expression in rat AC for control rats, vehicle-treated rats, and rats receiving 3,6′-dithiothalidomide;

FIG. 14A is a graph depicting the startle force of the startle-only condition for gap test performance in pre-blast rats, post-blast control rats, post-blast 3,6′-dithiothalidomide-treated rats, and sham-blast rats as assessed at 4, 8, 12, 16, 20, and 28 kHz and with BBN;

FIG. 14B is a graph depicting the startle force of the startle-only condition for prepulse inhibition (PPI) test performance in pre-blast rats, post-blast control rats, post-blast 3,6′-dithiothalidomide-treated rats, and sham-blast rats as assessed at 4, 8, 12, 16, 20, and 28 kHz and with BBN;

FIG. 15A is a graph depicting the gap ratio values (gap/startle-only response) for pre-blast rats, post-blast control rats, post-blast 3,6′-dithiothalidomide-treated rats, and sham-blast rats as assessed at 4, 8, 12, 16, 20, and 28 kHz and with BBN;

FIG. 15B is a graph depicting the PPI ratio values (PPI/startle-only response) for pre-blast rats, post-blast control rats, post-blast 3,6′-dithiothalidomide-treated rats, and sham-blast rats as assessed at 4, 8, 12, 16, 20, and 28 kHz and with BBN;

FIG. 16A is a graph depicting the tinnitus score for pre-blast rats, post-blast control rats, and post-blast 3,6′-dithiothalidomide-treated rats as assessed at 4, 8, 12, 16, 20, and 28 kHz and with BBN;

FIG. 16B is a graph depicting the tinnitus score for sham-blast rats as assessed at 4, 8, 12, 16, 20, and 28 kHz and with BBN;

FIG. 17A is a recording trace of an AC neuron depicting inhibitory synaptic currents in a sham-blast-exposed rat;

FIG. 17B is a recording trace of an AC neuron depicting inhibitory synaptic currents in a blast-exposed, vehicle-treated rat;

FIG. 17C is a recording trace of an AC neuron depicting inhibitory synaptic currents in a blast-exposed, 3,6′-dithiothalidomide-treated rat;

FIG. 18A is a graph depicting the miniature inhibitory postsynaptic current (mIPSC) amplitude of sham-blast-exposed (Control; Cont), blast-exposed vehicle-treated (Blast), and blast-exposed 3,6′-dithiothalidomide-treated (2-DT) rats;

FIG. 18B is a graph depicting the mIPSC frequency of sham-blast-exposed (Control; Cont), blast-exposed vehicle-treated (Blast), and blast-exposed 3,6′-dithiothalidomide-treated (2-DT) rats;

FIG. 19 is a graph depicting the cumulative distribution of mIPSC frequency of sham-blast-exposed (Sham), blast-exposed vehicle-treated (Blast), and blast-exposed 3,6′-dithiothalidomide-treated rats (3,6′-dithiothalidomide);

FIG. 20A is a graph depicting spontaneous bursting rates of DCN neurons in sham-blast-exposed (Control), blast-exposed vehicle-treated (Vehicle), and blast-exposed 3,6′-dithiothalidomide-treated (2-DT) rats as assessed at <10, 10-20, 20-30, and >30 kHz;

FIG. 20B is a graph depicting spontaneous firing rates of DCN neurons in sham-blast-exposed (Control), blast-exposed vehicle-treated (Vehicle), and blast-exposed 3,6′-dithiothalidomide-treated (2-DT) rats as assessed at <10, 10-20, 20-30, and >30 kHz;

FIG. 20C is a graph depicting a correlation between tinnitus score and the spontaneous bursting rate of DCN neurons from sham-blast-exposed rats (Control), blast-exposed vehicle-treated rats (Vehicle), and blast-exposed 3,6′-dithiothalidomide-treated (2-DT) rats;

FIG. 20D is a graph depicting a correlation between tinnitus score and the spontaneous firing rate of DCN neurons from sham-blast-exposed (Control), blast-exposed vehicle-treated (Vehicle), and blast-exposed 3,6′-dithiothalidomide-treated (2-DT) rats;

FIG. 21A is a graph depicting spontaneous bursting rates of AC neurons in sham-blast-exposed (Control), blast-exposed vehicle-treated (Vehicle), and blast-exposed 3,6′-dithiothalidomide-treated (2-DT) rats as assessed at 2-4, 4-16, and 16-42 kHz;

FIG. 21B is a graph depicting spontaneous firing rates of AC neurons in sham-blast-exposed (Control), blast-exposed vehicle-treated (Vehicle), and blast-exposed 3,6′-dithiothalidomide-treated (2-DT) rats as assessed at 2-4, 4-16, and 16-42 kHz;

FIG. 21C is a graph depicting a correlation between tinnitus score and the spontaneous bursting rate of AC neurons from sham-blast-exposed rats (Control), blast-exposed vehicle-treated rats (Vehicle), and blast-exposed 3,6′-dithiothalidomide-treated (2-DT) rats;

FIG. 21D is a graph depicting a correlation between tinnitus score and the spontaneous firing rate of AC neurons from sham-blast-exposed (Control), blast-exposed vehicle-treated (Vehicle), and blast-exposed 3,6′-dithiothalidomide-treated rats (2-DT);

FIG. 22A is a graph depicting the cross-correlation between recorded DCN neurons as assessed by correlogram ratio from sham-blast-exposed rats (Control), blast-exposed vehicle-treated rats (Vehicle), and blast-exposed 3,6′-dithiothalidomide-treated rats (2-DT) at <10, 10-20, 20-30, and >30 kHz;

FIG. 22B is a graph depicting the cross-correlation between recorded IC neurons as assessed by correlogram ratio from sham-blast-exposed rats (Control), blast-exposed vehicle-treated rats (Vehicle), and blast-exposed 3,6′-dithiothalidomide-treated rats (2-DT) at 2-4, 4-16, and 16-42 kHz;

FIG. 22C is a graph depicting the cross-correlation between recorded AC neurons as assessed by correlogram ratio from sham-blast-exposed rats (Control), blast-exposed vehicle-treated rats (Vehicle), and blast-exposed 3,6′-dithiothalidomide-treated rats (2-DT) at 2-4, 4-16, and 16-42 kHz;

FIG. 23A is a graph depicting the quantification of ionized calcium-binding adapter molecule 1 (Iba-1) expression from immune-stained slides of rat microglia from sham-blast-exposed rats (Control), blast-exposed vehicle-treated rats (Vehicle), and blast-exposed 3,6′-dithiothalidomide-treated rats (2-DT);

FIG. 23B is a graph depicting the quantification of TNF-α expression from immune-stained slides of rat microglia from sham-blast-exposed rats (Control), blast-exposed vehicle-treated rats (Vehicle), and blast-exposed 3,6′-dithiothalidomide-treated rats (2-DT);

FIG. 24A is a graph depicting the quantification of TNF-α expression from immune-stained slides of rat astrocytes from sham-blast-exposed rats (Control), blast-exposed vehicle-treated rats (Vehicle), and blast-exposed 3,6′-dithiothalidomide-treated rats (2-DT);

FIG. 24B is a graph depicting the quantification of glial fibrillary acidic protein (GFAP) expression from immune-stained slides of rat astrocytes from sham-blast-exposed rats (Control), blast-exposed vehicle-treated rats (Vehicle), and blast-exposed 3,6′-dithiothalidomide-treated rats (2-DT);

FIG. 25 is a graph depicting the startle-response ratio at 7, 10, 14, and 21 kHz of 3,6′-dithiothalidomide-treated mice before and after exposure to 123 decibel (dB) noise;

FIG. 26A is a graph depicting the mIPSC frequency of mouse neurons in control WT mice, WT mice exposed to 123 db noise, and 3,6′-dithiothalidomide-treated WT mice exposed to 123 db noise;

FIG. 26B is a graph depicting the mIPSC amplitude of mouse neurons in control WT mice, WT mice exposed to 123 db noise, and 3,6′-dithiothalidomide-treated WT mice exposed to 123 db noise;

FIG. 26C is a graph depicting the miniature excitatory synaptic current (mEPSC) frequency of mouse neurons in control WT mice, WT mice exposed to 123 db noise, and 3,6′-dithiothalidomide-treated WT mice exposed to 123 db noise;

FIG. 26D is a graph depicting the mEPSC amplitude of mouse neurons in control WT mice, WT mice exposed to 123 db noise, and 3,6′-dithiothalidomide-treated WT mice exposed to 123 db noise;

FIG. 27 is a graph depicting the gap ratio values (gap/startle-only response) for pre-blast and sham-blast rats as assessed at 4, 8, 12, 16, 20, and 28 kHz and with BBN;

FIG. 28 is a graph depicting the gap ratio values (gap/startle-only response) for pre-blast and post-blast vehicle-treated rats as assessed at 4, 8, 12, 16, 20, and 28 kHz and with BBN; and

FIG. 29 is a graph depicting the gap ratio values (gap/startle-only response) for pre-blast and post-blast etanercept-treated rats as assessed at 4, 8, 12, 16, 20, and 28 kHz and with BBN.

### DEFINITIONS

As used herein, the term “hearing disorder associated with maladaptive neuroplasticity, reduction of inhibition, shift of excitation-to-inhibition balance, and/or changes in central gain and neural sensitivity” refers to tinnitus, hyperacusis and auditory processing deficit/disorder (APD). The term “maladaptive neuroplasticity, reduction of inhibition, shift of excitation-to-inhibition balance, and/or changes in central gain and neural sensitivity,” as used herein, refers to perceptual correlates of cortical reorganization.

As used herein, the term “tinnitus” refers to a hearing disorder wherein a subject experiences or has the sensation of sound in one or both ears, such as buzzing, ringing, whistling, hissing or booming occurring without an external stimulus. Tinnitus is commonly a secondary condition caused by a specific primary condition and may be referred as secondary tinnitus. Common primary conditions that lead to tinnitus include but are not limited to, e.g., exposure to loud noise, blast exposure, trauma, head trauma, inner ear infection, middle ear infection, allergic reaction, inner ear tumor, middle ear tumor, otosclerosis, aging, Meniere's disease, high blood pressure, low blood pressure, anemia, diabetes, thyroid dysfunction, glucose metabolism abnormalities, vascular disorders, growth on the jugular vein, acoustic tumors, aneurysms, head aneurysms, and neck aneurysms. In some instances, tinnitus may be the result of exposure to particular chemical or drugs and may be referred to as chemical or drug-induced tinnitus. For example, drug exposure that has been associated with tinnitus include but are not limited to, e.g., exposure to aspirin, ibuprofen, nonsteroidal anti-inflammatory drugs (NSAIDs), quinine, sedatives, antidepressants, antibiotics and chemotherapeutic agents.

As used herein, “auditory processing deficit” (APD) refers to a complex hearing disorder characterized by the impairment of a human in understanding spoken language even though the human can hear. APD may be caused by blast trauma and trauma caused, for example, by automobile accidents. Symptoms of APD may include difficulty in understanding speech in noisy environments, following directions, and distinguishing between similar sounds; breakdown in the ability to process auditory input resulting in difficulty listening and communicating, especially in the academic setting; poor performance in sound localization and lateralization, auditory discrimination, auditory pattern recognition, temporal aspects of audition (including temporal integration, temporal discrimination (e.g., temporal gap detection), temporal ordering, and temporal masking), auditory performance in competing acoustic signals (including dichotic listening), and auditory performance with degraded acoustic signals. The APD may be central APD (CAPD). The APD may be noise-related APD, noise-related CAPD, blast trauma-related APD, or blast trauma-related CAPD.

As used herein, “hyperacusis” refers to a hearing disorder characterized by an increased sensitivity to certain frequency and volume ranges of sound. Severe hyperacusis may result in difficulty tolerating everyday sounds, which may seem unpleasantly or painfully loud to a person presenting with hyperacusis but not to others.

As used herein, the terms “inhibit” and “block” are used interchangeably and refer to the function of a particular agent to effectively impede, retard, arrest, suppress, prevent, decrease, or limit the function or action of another agent or agents or cell or cells or cellular process or cellular processes. In such instances, an agent that inhibits is referred to as an “inhibitor,” which term is used interchangeably with “inhibitory agent” and “antagonist.” As used herein, the term “inhibitor” refers to any substance or agent that interferes with or slows or stops a chemical reaction, a signaling reaction, or other biological or physiological activity. An inhibitor may be a direct inhibitor that directly binds the substance or a portion of the substance that it inhibits, or it may be an indirect inhibitor that inhibits through an intermediate function, e.g., through binding of the inhibitor to an intermediate substance or agent that subsequently inhibits a target.

As used herein the term “small molecule” refers to a small organic or inorganic compound having a molecular weight of more than 50 and less than about 2,500 daltons. Small molecule agents may include functional groups necessary for structural interaction with proteins, particularly hydrogen bonding, and may include at least an amine, carbonyl, hydroxyl or carboxyl group, and may contain at least two of the functional chemical groups. The small molecule agents may include cyclical carbon or heterocyclic structures and/or aromatic or polyaromatic structures substituted with one or more functional groups. Small molecule agents are also found among biomolecules including peptides, saccharides, fatty acids, steroids, purines, pyrimidines, derivatives, structural analogs or combinations thereof.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a hearing disorder and/or adverse effect attributable to the hearing disorder. “Treatment,” as used herein, covers any treatment of a hearing disorder associated with maladaptive neuroplasticity, reduction of inhibition, shift of excitation-to-inhibition balance, changes in central gain, and/or changes in neural sensitivity in a mammal, particularly in a human, and includes: (a) preventing the hearing disorder associated with maladaptive neuroplasticity, reduction of inhibition, shift of excitation-to-inhibition balance, changes in central gain, and/or changes in neural sensitivity from occurring in a subject which may be predisposed to the hearing disorder associated with maladaptive neuroplasticity, reduction of inhibition, shift of excitation-to-inhibition balance, changes in central gain, and/or changes in neural sensitivity but has not yet been diagnosed as having it; (b) inhibiting the hearing disorder associated with maladaptive neuroplasticity, reduction of inhibition, shift of excitation-to-inhibition balance, and/or changes in central gain and neural sensitivity, i.e., arresting its progression; and (c) relieving the hearing disorder associated with maladaptive neuroplasticity, reduction of inhibition, shift of excitation-to-inhibition balance, and/or changes in central gain and neural sensitivity, i.e., causing regression of the hearing disorder associated with maladaptive neuroplasticity, reduction of inhibition, shift of excitation-to-inhibition balance, and/or changes in central gain and neural sensitivity.

The terms “individual,” “subject,” “host,” and “patient,” used interchangeably herein, refer to a mammal, including, but not limited to, rodents such as murines, rabbits, simians, humans, mammalian farm animals, mammalian sport animals, and mammalian pets.

A “therapeutically effective amount” or “efficacious amount” means the amount of a compound that, when administered to a mammal or other subject for treating a hearing disorder, is sufficient to effect such treatment for the hearing disorder. The “therapeutically effective amount” will vary depending on the compound, the hearing disorder and its severity and the age, weight, etc., of the subject to be treated.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle.

The terms “sample,” “patient sample” and “biological sample” are used interchangeably and encompass a variety of sample types obtained from an individual and can be used in a diagnostic or monitoring assay. The definition encompasses blood, serum, cerebral spinal fluid and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides or polypeptides. The term “biological sample” encompasses a clinical sample, and also includes cells in culture, cell supernatants, cell lysates, serum, plasma, biological fluid, and tissue samples. In some embodiments, a biological sample will include cells (e.g., blood cells, immune cells, skin cells, etc.)

As used herein, the phrase “pharmaceutically acceptable carrier” refers to a carrier medium that does not interfere with the effectiveness of the biological activity of the active ingredient. Such a carrier medium is essentially chemically inert and nontoxic.

As used herein, the phrase “pharmaceutically acceptable” means approved by a regulatory agency of the Federal government or a state government, or listed in the U.S. Pharmacopeia or other generally recognized pharmacopeia for use in animals, and more particularly for use in humans.

As used herein, the term “carrier” refers to a diluent, adjuvant, excipient, or vehicle with which the therapeutic is administered. Such carriers can be sterile liquids, such as saline solutions in water, or oils, including those of petroleum, animal, vegetable or synthetic origin, such as peanut oil, soybean oil, mineral oil, sesame oil and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The carrier, if desired, can also contain minor amounts of wetting or emulsifying agents, or pH buffering agents. These pharmaceutical compositions can take the form of solutions, suspensions, emulsion, tablets, pills, capsules, powders, sustained-release formulations and the like. The composition can be formulated as a suppository, with traditional binders and carriers such as triglycerides. Examples of suitable pharmaceutical carriers are described in Remington's Pharmaceutical Sciences by E. W. Martin. Examples of suitable pharmaceutical carriers are a variety of cationic polyamines and lipids, including, but not limited to N-(1(2,3-dioleyloxy)propyl)-N,N,N-trimethylammonium chloride (DOTMA) and diolesylphosphotidylethanolamine (DOPE). Liposomes are suitable carriers for gene therapy uses of the present disclosure. Such pharmaceutical compositions should contain a therapeutically effective amount of the compound, together with a suitable amount of carrier so as to provide the form for proper administration to the subject. The formulation should suit the mode of administration.

The terms “polypeptide,” “peptide,” and “protein,” used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like. The term also includes antibodies which are further described herein.

As used herein, the tem “antibody” is intended to refer to immunoglobulin molecules having four polypeptide chains, two heavy (H) chains and two light (L) chains inter-connected by disulfide bonds. Each chain consists of a variable portion, denoted V_{H} and V_{L} for variable heavy and variable light portions, respectively, and a constant region, denoted C_{H} and C_{L} for constant heavy and constant light portions, respectively. The C_{H} portion contains three domains CH1, CH2, and CH3. Each variable portion is composed of three hypervariable complementarity determining regions (CDRs) and four framework regions (FRs).

The term “antibody” also encompasses antibody fragments, such as (i) a Fab fragment, which is a monovalent fragment consisting of the V_{L}, V_{H}, C_{L} and C_{H}1 domains; (ii) a F(ab′)₂ fragment, a bivalent fragment comprising two Fab fragments linked by a disulfide bridge at the hinge region; (iii) an Fd fragment consisting of the V_{H} and C_{H}1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). Furthermore, although the two domains of the Fv fragment, V_{L} and V_{H}, are coded for by separate genes, they can be joined by recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). The term antibody also encompasses antibodies having this scFv format.

The term “human antibody,” as used herein, is intended to include antibodies having variable and constant regions derived from human immunoglobulin sequences. Methods of making human antibodies include, but are not limited to, those described by Duvall et al. (2011) MAbs 3(2): 203-208 and Traggiai et al. (2004) Nat Med 10(8):871-5, the disclosures of which are incorporated herein by reference.

The term “chimeric” antibody as used herein refers to an antibody having variable sequences derived from a non-human immunoglobulin, such as rat or mouse or primate antibody, and human immunoglobulin constant regions, in some instances chosen from a human immunoglobulin template. Methods for producing chimeric antibodies are known in the art. See, e.g., Morrison, 1985, Science 229(4719):1202-7; Oi et al., 1986, BioTechniques 4:214-221; Gillies et al., 1985, J. Immunol. Methods 125:191-202; U.S. Pat. Nos. 5,807,715; 4,816,567; and 4,816,397, which are incorporated herein by reference in their entireties.

The term “humanized antibody” is intended to include antibodies in which one or more of the regions or domains of the antibody is derived from a non-human source, e.g., an antibody in which one of the heavy- or light-chain CDRs is derived from a mouse anti-TNF-α antibody, that is, has the same coding sequence or the same amino acid sequence or a sequence more closely related to a mouse anti-TNF-α than to a human anti-TNF-α antibody, and one or more regions or domains derived from a human source. The relative contribution of the human and the non-human sources in the construction of a humanized antibody will vary, and in some instances the resulting humanized antibody will range from as much as 60% to 99% human, including but not limited to, e.g., as much as 70% human, as much as 80% human, as much as 85% human, as much as 90% human, and as much as 95% human. Methods of making humanized antibodies include, but are not limited to, those described by Jones et al. (1986) Nature 321(6069):522-5 and Chames et al. (2009) Br J Pharmacol 157:220-233, the disclosures of which are incorporated herein by reference.

The term “primatized antibody” refers to an antibody comprising monkey variable regions and human constant regions. Methods for producing primatized antibodies are known in the art. See e.g., U.S. Pat. Nos. 5,658,570; 5,681,722; and 5,693,780, which are incorporated herein by reference in their entireties.

A “neutralizing antibody,” as used herein refers to an antibody whose binding to TNF-α results in the inhibition of the biological activity of TNF-α, as assessed by measuring one or more indicators of TNF-α, such as TNF-α-induced cellular activation or TNF-α binding to one or more TNF-α receptors or TNF-α signaling or the response of a TNF-α reporter, etc. These indicators of biological activity can be assessed by standard in vitro or in vivo assays known in the art.

The term “monoclonal antibody” as used herein is not limited to antibodies produced through hybridoma technology. The term “monoclonal antibody” refers to an antibody that is derived from a single clone, including any eukaryotic, prokaryotic, or phage clone and not the method by which it is produced. Monoclonal antibodies useful in connection with the present disclosure can be prepared using a wide variety of techniques including, but not limited to, the use of hybridoma, recombinant, and phage display technologies or a combination thereof. The anti-TNF-α monoclonal antibodies, as described herein, include, but are not limited to, chimeric, primatized, humanized, or human antibodies.

The terms “recombinant polypeptide,” “recombinant peptide,” “recombinant binding protein” and “recombinant antibody,” as used herein, are intended to include all polypeptides, peptides, binding proteins and antibodies that are prepared, expressed, created or isolated by recombinant means, such as polypeptides, peptides, binding proteins and antibodies expressed using a recombinant expression vector transfected into a host cell.

The terms “isolated polypeptide,” “isolated peptide,” “isolated binding protein” and “isolated antibody,” as used herein, are intended to refer to a polypeptide, peptide, binding protein and/or antibody that is substantially free of other polypeptides, peptides, binding proteins and/or antibodies.

The teens “nucleic acid,” “nucleic acid molecule” and “polynucleotide” are used interchangeably and refer to a polymeric form of nucleotides of any length, either deoxyribonucleotides or ribonucleotides, or analogs thereof. Non-limiting examples of nucleic acids and polynucleotides include linear and circular nucleic acids, messenger RNA (mRNA), cDNA, recombinant polynucleotides, vectors, probes, primers, single-, double-, or multi-stranded DNA or RNA, genomic DNA, DNA-RNA hybrids, chemically or biochemically modified, non-natural or derivatized nucleotide bases, oligonucleotides containing modified or non-natural nucleotide bases (e.g., locked-nucleic acid (LNA) oligonucleotides), and interfering RNAs.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence similarity can be determined in a number of different manners. To determine sequence identity, sequences can be aligned using the methods and computer programs, including BLAST, available over the world wide web at ncbi(dot)nlm(dot)nih(dot)gov/BLAST. See, e.g., Altschul et al. (1990), J. Mol. Biol. 215:403-10. Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

The terms “double stranded RNA,” “dsRNA,” “partial-length dsRNA,” “full-length dsRNA,” “synthetic dsRNA,” “in vitro produced dsRNA,” “in vivo produced dsRNA,” “bacterially produced dsRNA,” “isolated dsRNA,” and “purified dsRNA” as used herein refer to nucleic acid molecules capable of being processed to produce a smaller nucleic acid, e.g., a short interfering RNA (siRNA), capable of inhibiting or down regulating gene expression, for example by mediating RNA interference “RNAi” or gene silencing in a sequence-specific manner. Design of a dsRNA or a construct comprising a dsRNA targeted to a gene of interest is routine in the art, see e.g., Timmons et al. (2001) Gene, 263:103-112; Newmark et al. (2003) Proc Natl Acad Sci USA, 100 Supp 1:11861-5; Reddien et al. (2005) Developmental Cell, 8:635-649; Chuang & Meyerowitz (2000) Proc Natl Acad Sci USA, 97:4985-90; Piccin et al. (2001) Nucleic Acid Res, 29:E55-5; Kondo et al. (2006) Genes Genet Syst, 81:129-34; and Lu et al. (2009) FEBS J, 276:3110-23; the disclosures of which are incorporated herein by reference.

The terms “short interfering RNA,” “siRNA”, and “short interfering nucleic acid” are used interchangeably and may refer to short hairpin RNA (shRNA), short interfering oligonucleotide, short interfering nucleic acid, short interfering modified oligonucleotide, chemically-modified siRNA, post-transcriptional gene silencing RNA (ptgsRNA), and other short oligonucleotides useful in mediating an RNAi response. In some instances, siRNA may be encoded from DNA comprising a siRNA sequence in vitro or in vivo as described herein. When a particular siRNA is described herein, it will be clear to the ordinary skilled artisan as to where and when a different but equivalently effective interfering nucleic acid may be substituted, e.g., the substation of a short interfering oligonucleotide for a described shRNA and the like.

The term “gene” refers to a particular unit of heredity present at a particular locus within the genetic component of an organism. A gene may be a nucleic acid sequence, e.g., a DNA or RNA sequence, present in a nucleic acid genome, a DNA or RNA genome, of an organism and, in some instances, may be present on a chromosome. Typically, a gene will be a DNA sequence that encodes for an mRNA that encodes a protein. A gene may include a single exon and no introns or multiple exons and one or more introns. One of two or more identical or alternative forms of a gene present at a particular locus is referred to as an “allele” and, for example, a diploid organism will typically have two alleles of a particular gene. New alleles of a particular gene may be generated either naturally or artificially through natural or induced mutation and propagated through breeding or cloning. A gene or allele may be isolated from the genome of an organism and replicated and/or manipulated or a gene or allele may be modified in situ through gene therapy methods. The locus of a gene or allele may have associated regulatory elements and gene therapy, in some instances, may include modification of the regulatory elements of a gene or allele while leaving the coding sequences of the gene or allele unmodified.

The terms “tumor necrosis factor,” “tumor necrosis factor alpha,” “TNF-α,” “TNF-alpha,” and “pro-TNF-α” are used interchangeably herein, except where specified, and refer to the cytokine polypeptide encoded from the TNF-α genomic locus in all processed, modified, unprocessed, and unmodified forms. TNF-α is also known as Cachectin and Tumor necrosis factor ligand superfamily member 2 (TNFSF2). The human TNF-α polypeptide and fragments thereof are represented by GenBank Accession: CAA26669.1, AAH28148.1, CAB63905.1, and CAB63904.1; UniProt/Swiss-Prot. ID: P01375.1; and NCBI Ref Seq.: NP_000585.2. “TNF-α receptors” are those cellular receptors to which TNF-α binds and include but are not limited to human TNFRSF1A/TNFR1 and human TNFRSF1B/TNFBR which are represented by GenBank Accession Nos: AAO23979.1, AAH10140.1, EAW88806.1, EAW88805.1, AAM77802.1, ACH57451.1, AAH11844.1, AAH52977.1, EAW71735.1, EAW71734.1, EAW71733.1, EAW71732.1, EAW71731.1, AAP88939.1, AAO89076.1; UniProt/Swiss-Prot. ID: P19438.1, P20333.3; and NCBI Ref. Seq.: NP_001056.1, NP_001057.1.

The term “TNF-α gene” refers to the nucleic acid sequence from which TNF-α is produced. A TNF-α gene may refer to the naturally occurring TNF-α locus present within an organism's genome or may refer to an isolated version of the TNF-α gene, or fragments thereof, that has been removed or copied from the genome of an organism. In some instances, the term may refer to synthetic or modified versions of a TNF-α gene that have been altered in vitro or in vivo, e.g., through recombinant methods. The human TNF-α gene is encoded from the TNF-α gene locus present at genomic location 6p21.3 (NCBI Reference Sequence: NG_007462.1, Gene ID: 7124, Locus Tag: DADB-70P7.1).

The terms “TNF-α signaling,” “TNF-α signaling pathway,” “TNF-α pathway members” and “TNF-α pathway” refer to genes and gene products that are upregulated, down-regulated, or caused to be modified by the signaling of TNF-α through one or more TNF-α receptors. For example, a TNF-α signaling pathway gene or a TNF-α pathway member may be a gene that is upregulated or down-regulated when TNF-α is bound to its receptor relative to the expression level of the gene when TNF-α is not bound to its receptor. Likewise, e.g., a TNF-α pathway member may be a protein that is modified, e.g., phosphorylated, when TNF-α is bound to its receptor relative to the state of modification, e.g., phosphorylation state, of the protein when TNF-α is not bound to its receptor.

TNF-α signaling pathway members include but are not limited to, e.g., TRAF1, CUL1, TNFRSF1A, MAP3K8, TNFRSF1B, PPP2CA, TRADD, RIPK3, TRAF2, CASP9, Pro-CASP8, NFKB1, MAP4K2, CSNK2A1, MAP2K4, CASP3, MAP2K7, NOXO1, MAPK8, BID, MAPK9, SELE, JUN, NFKBIE, MAP3K5, MAP3K3, TXN, CREBBP, MAP2K3, TANK, MAP2K6, BIRC2, MAPK3, OTUD7B, MAPK1, CYBA, GRB2, HSP90AA1, SOS1, IL6, RAF1, BAD, RAS, GLUL, NSMAF, TNF, SMPD2, FBXW11, PSMD2, REL, TRAP1, NFKB2, MADD, BTRC, KSR1, PRKCZ, KSR2, MAP3K1, FADD, TBK1, CASP8, PLK1, CFLAR, TNFAIP3, MAP3K14, BIRC3, RIPK1, DIABLO, MAP3K7, RFFL, TAB1, RFK, TAB2, NOX1, TAB3, RAC1, CHUK, CDC37, IKBKB, RELA, IKBKG, BCL2L1, AKT1, BAX, NFKBIB, APAF1, RELA, CASP7, NFKBIA, PYGL, SKP1, and CCL2. TNF-α signaling is described in Baud & Karin (2001) Trends Cell Biol 11(9):372-7, Olmos & Llado (2014) Mediators Inflamm 2014:861231, Shubayev et al. Chapter 8: Cytokines in Pain (2010) Kruger & Light Translational Pain Research: From Mouse to Man: CRC Press, Boca Raton, Fla., the disclosures of which are incorporated herein by reference.

The term “non-TNF-α gene” and “non-TNF-α pathway genes” as used herein refer to genes and genes which encode gene products that are not generally associated with TNF-α signaling or activation, upregulation, down-regulation, or inhibition upon the binding of TNF-α to one or more TNF-α receptors. Non-TNF-α genes are essentially not affected by TNF-α signaling or are not specifically or directly affected by TNF-α signaling. By “not specifically or directly affected by,” it is meant that the non-TNF-α gene is not more or not significantly more affected by TNF-α signaling than the majority of other genes generally not associated with TNF-α signaling.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a TNFα inhibitor” includes a plurality of such TNFα inhibitors and reference to “the TNFα inhibitor” includes reference to one or more TNFα inhibitors and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION OF THE INVENTION

Provided herein are methods of treating a subject having a hearing disorder associated with maladaptive neuroplasticity, reduction of inhibition, shift of excitation-to-inhibition balance, changes in central gain, and/or changes in neural sensitivity through inhibition of TNF-α, TNF-α signaling, and related molecules of the TNF-α pathway. Also, provided are methods of treating a subject having a hearing disorder associated with maladaptive neuroplasticity, reduction of inhibition, shift of excitation-to-inhibition balance, changes in central gain, and/or changes in neural sensitivity by inhibiting TNF-α and/or TNF-α signaling through endogenous disruption of the an allele of a TNF-α gene or a gene of the TNF-α signaling pathway.

In particular aspects, the inventive methods relate to the treatment of a hearing disorder associated with maladaptive neuroplasticity, reduction of inhibition, shift of excitation-to-inhibition balance, and/or changes in central gain and neural sensitivity, wherein the hearing disorder associated with maladaptive neuroplasticity, reduction of inhibition, shift of excitation-to-inhibition balance, changes in central gain, and/or changes in neural sensitivity is tinnitus, hyperacusis, or adaptive processing deficit (ADP). In particular aspects of the invention, a subject has a hearing disorder associated with maladaptive neuroplasticity, reduction of inhibition, shift of excitation-to-inhibition balance, changes in central gain, and/or changes in neural sensitivity and yet the subject does not present with hearing loss. In particular aspects of the invention, a subject has a hearing disorder associated with maladaptive neuroplasticity, reduction of inhibition, shift of excitation-to-inhibition balance, changes in central gain, and/or changes in neural sensitivity and the subject does not present with cochlear hearing loss. For example, various aspects of the invention relate to methods of treating a subject that has tinnitus, hyperacusis, or ADP, and does not have cochlear hearing loss.

### Methods

Aspects of the disclosure include methods of treating a subject for a hearing disorder associated with maladaptive neuroplasticity, reduction of inhibition, shift of excitation-to-inhibition balance, changes in central gain, and/or changes in neural sensitivity through the administration of one or more TNF-α inhibitory agents.

### TNF-α and TNF-α Signaling

TNF-α and TNF-α signaling are well understood in the art and described in, e.g., Palladino et al. (2003) Nat Rev Drug Discov. 2(9):736-46; Barbara et al. (1996) Immunol Cell Biol. 74(5):434-43; Pickering et al. (1996) Immunol Cell Biol. 74(5):434-43; Pennica et al. (1984) Nature 312:724-729; Davis et al. (1987) Biochemistry 26:1322-1326; and Jones et al. (1989) Nature 338:225-228, the disclosures of which are incorporated herein by reference. Briefly, human TNF-α is translated as a 26-kDa protein that lacks a classic signal peptide. Synthesized pro-TNF-α expressed on the plasma membrane is cleaved through the action of matrix metalloproteinases to release a mature soluble 17-kDa TNF-α. In both its cell-associated and secreted forms, trimerization is required for biological activity. Both the cell-associated 26-kDa and secreted 17-kDa forms are biologically active. Cell-associated TNF-α is processed to a secreted form by TNF-α-converting enzyme (TACE; also referred to as ADAM-17).

The biological response to TNF-α and TNF-α signaling is mediated through receptors. Receptors for TNF-α include transmembrane glycoproteins with multiple cysteine rich repeats in the extracellular N-terminal domains, e.g., type I receptors, e.g., Tumor Necrosis Factor Receptor 1 (TNFR1, a.k.a. p60, p55, CD120a), and type II receptors, e.g., Tumor Necrosis Factor Receptor 2 (TNFR2, a.k.a. p80, p75, CD120b). TNF-α signaling through TNFR1 and TNFR2 may be either overlapping or distinct.

### TNF-α Inhibitory Agents

TNF-α inhibitory agents may be any agent known to inhibit the function, production, or biological availability of TNF-α or the downstream signaling of the TNF-α pathway.

In some instances, TNF-α inhibitory agents are agents that directly bind TNF-α. TNF-α inhibitory agents that directly bind to TNF-α may inhibit various functions of TNF-α including, but not limited to, binding of TNF-α to a TNF-α receptor, binding of TNF-α to TNF-α (e.g., trimerization), binding of TNF-α processing agents thus inhibiting processing of TNF-α (e.g., pro-TNF-α processing, TACE TNF-α processing, etc.), binding of TNF-α cleaving agents thus inhibiting cleaving of TNF-α (e.g., cleaving of TNF-α at the cell membrane, metalloproteinases release of TNF-α, etc.), and the like. In other instances, TNF-α inhibitory agents are agents that directly bind TNF-α may prevent TNF-α from being expressed on the cell surface, e.g., by preventing newly translated TNF-α from being transported to the cell membrane or by preventing modification of TNF-α that allows TNF-α to be expressed on the membrane.

In some instances, a TNF-α inhibitory agent may interfere, directly or indirectly, with proteolytic processing of TNF-α. For example, a TNF-α inhibitory agent may interfere with proteolytic processing, including but not limited to, proteolytic processing of TNF-α by metalloproteinases, proteolytic processing of TNF-α by TACE, proteolytic processing of TNF-α by signal peptide peptidase-like 2A (SPPL2A), proteolytic processing of TNF-α by signal peptide peptidase-like 2B (SPPL2B), etc.

In some instances, a TNF-α inhibitory agent may preferentially target either soluble or membrane tethered TNF-α. For example, in some instances, a TNF-α inhibitory agent may preferentially bind soluble TNF-α. In other instances, a TNF-α inhibitory agent may preferentially bind membrane tethered TNF-α. In other instances, a TNF-α inhibitory agent may preferentially prevent the production of soluble TNF-α. In yet other instances, instances of a TNF-α inhibitory agent may preferentially prevent the production of membrane tethered TNF-α. In certain embodiments, a TNF-α inhibitory agent may preferentially prevent the function of soluble TNF-α. In some embodiments, a TNF-α inhibitory agent may preferentially prevent the function of membrane tethered TNF-α.

In some instances, a TNF-α inhibitory agent may interfere, directly or indirectly, with post-translational modification of TNF-α and thus inhibit TNF-α function. For example, a TNF-α inhibitory agent may interfere with or prevent TNF-α phosphorylation, e.g., phosphorylation on serine residues, including but not limited to preventing phosphorylation of membrane bound TNF-α. A TNF-α inhibitory agent may interfere with or prevent TNF-α dephosphorylation, e.g., dephosphorylation of serine residues, including but not limited to preventing dephosphorylation of membrane bound TNF-α. In other instances, a TNF-α inhibitory agent may interfere with other post-translational modifications of TNF-α or the reversal of other post-translational modifications of TNF-α, including but not limited to, glycosylation, including but not limited to O-linked glycosylation, N-linked glycosylation, fatty acid acylation, the removal of fatty acids, and the like.

In some instances, TNF-α inhibitory agents are agents that directly bind a TNF-α receptor and antagonize binding of TNF-α to a TNF-α receptor. Binding of a TNF-α inhibitory agent to a TNF-α receptor may block TNF-α signaling through means other than preventing TNF-α from binding its receptor including, e.g., preventing signal transduction.

In some instances, a TNF-α inhibitory agent may decrease the effective concentration of soluble TNF-α. For example, in some instances, a TNF-α inhibitory agent may be a soluble form of or a solubilized portion of a TNF-α receptor. Such agents that decrease the effective concentration of soluble TNF-α bind or sequester soluble TNF-α without activating TNF-α signaling thus decreasing the amount of free soluble TNF-α available to bind TNF-α receptors capable of activating TNF-α signaling.

In some instances, a TNF-α inhibitory agent may be an antibody or fragment thereof that directly binds to TNF-α or a TNF-α receptor, including but not limited to, e.g., an isolated antibody, a recombinant antibody, a neutralizing antibody, a humanized antibody, a human antibody, a Fab fragment, a F(ab)₂ fragment, a Fd fragment, a Fv fragment, a scFv antibody, and the like.

In certain embodiments, a TNF-α inhibitory agent useful in the methods presented herein may be a commercially available TNF-α antibody. Any convenient commercially available TNF-α antibody may be employed, including but not limited to, e.g., infliximab (REMICADE®, Janssen Biotech, Horsham, Pa.), a chimeric antibody having murine anti-TNF-α variable domains and human IgG₁ constant domains; adalimumab (HUMIRA®, Abbott Laboratories, Abbott Park, Ill.), a recombinant, fully human anti-TNF-α antibody that binds specifically to TNF-α and blocks its interaction with TNF-α receptors; certolizumab pegol (Cimzia®); folimumab (Simponi®); CDP-571 (Humicade™), D2E7, CDP-870, and the like. In other instances, anti-TNF-α antibodies and TNF-α binding proteins useful in practicing the methods presented herein may include those antibodies and binding proteins described in U.S. Pat. Nos. 8,722,860, 7,981,414 and 6,090,382, U.S. Patent Pub. Nos. 2006/0024308 and 2004/0033228, and PCT Pub. Nos. WO2002080892A1, WO2006014477A1 and WO2013063114A1, the disclosures of which are incorporated herein by reference.

In certain embodiments, a TNF-α inhibitory agent useful in the methods presented herein may be curcumin or a catechin.

In certain embodiments, a TNF-α inhibitory agent useful in the methods presented herein may be a commercially available TNF-α soluble receptor. Any convenient commercially available TNF-α soluble receptor may be employed, including but not limited to, e.g., etanercept (ENBREL®, Amgen Inc., Thousand Oaks, Calif.), a recombinant fusion protein comprising two p75 soluble TNF-receptor domains linked to the Fc portion of a human immunoglobulin; lenercept, pegylated TNF-receptor type I, TBP-1, and the like.

In some embodiments, a TNF-α inhibitory agent useful in the methods presented herein may be an engineered TNF-α molecule. Such engineered TNF-α molecules are known in the art and include, but are not limited to, engineered TNF-α molecules which form trimers with native TNF-α and prevent receptor binding (see, e.g., Steed et al. (2003) Science 301:1895-1898, WO 03/033720, and WO 01/64889, the disclosures of which are incorporated herein by reference).

Such TNF-α inhibitory agents and methods for their use are discussed in, e.g., Weinberg & Buchholz. TNF-α Inhibitors: Milestones in Drug Therapy (2006) Springer Science & Business Media, the disclosure of which is incorporated herein by reference.

In certain embodiments, a TNF-α inhibitory agent useful in the methods presented herein may be a small molecule TNF-α inhibitor. Such small molecule TNF-α inhibitors may be specific or non-specific TNF-α inhibitors and include but are not limited to, e.g., MMP inhibitors (i.e. matrix metalloproteinase inhibitors), TACE inhibitors (i.e. TNF Alpha Converting Enzyme inhibitors), tetracyclines (e.g., doxycycline, lymecycline, oxitetracycline, tetracycline, minocycline and synthetic tetracycline derivatives, such as chemically modified tetracyclines), prinomastat (AG3340), batimastat, marimastat, BB-3644, KB-R7785, quinolones (e.g., norfloxacin, levofloxacin, enoxacin, sparfloxacin, temafioxacin, moxifloxacin, gatifloxacin, gemifloxacin, grepafloxacin, trovafloxacin, ofloxacin, ciprofloxacin, refloxacin, lomefloxacin, temafioxacin etc.), thalidomide, thalidomide derivatives, 3,6′-dithiothalidomide, selective cytokine inhibitors, CC-1088, CDC-501, CDC-801, Linomide (Roquininex®), lazaroids, non-glucocorticoid 21-aminosteroids (e.g., U-74389G (16-desmethyl tirilazad) and U-74500), cyclosporin, pentoxifyllin derivates, hydroxamic acid derivates, napthopyrans, phosphodiesterase I, II, III, IV, and V-inhibitors; CC-1088, Ro 20-1724, rolipram, amrinone, pimobendan, vesnarinone, SB 207499 (Ariflo®), melancortin agonists, HP-228, CT3, ITF-2357, PD-168787, CLX-1100, M-PGA, NCS-700, PMS-601, RDP-58, TNF-484A, PCM-4, CBP-1011, SR-31747, AGT-1, solimastat, CH-3697, NR58-3.14.3, RIP-3, Sch-23863, iloprost, prostacyclin, CDC-801 (Celgene), DPC-333 (Dupont), VX-745 (Vertex), AGIX-4207 (AtheroGenics), ITF-2357 (Italfarmaco), and the like.

In certain embodiments, the TNF-α inhibitory agent is thalidomide or a derivative or analog thereof, including but not limited to, e.g., those described in Muller et al. (1996) J Med Chem 39(17):3238-40, the disclosure of which is incorporated herein by reference. In some instances, the TNF-α inhibitory agent is an immune-modulatory drug or a derivative or analog thereof of which thalidomide is a non-limiting example. Other immune-modulatory drugs useful as a TNF-α inhibitory agent according to the methods described herein include but are not limited to, e.g., lenalidomide and pomalidomide. The mechanisms through which thalidomide, and derivatives or analogs thereof, and immune-modulatory drugs, and derivatives or analogs thereof, inhibit TNF-α and/or TNF-α signaling are described in, e.g., Muller et al. (1996) J Med Chem 39(17):3238-40; Lopez-Girona et al. (2012) Leukemia 26(11): 2326-2335; Zhu et al. (2013) Leuk Lymphoma 54(4):683-7; Majumder et al. (2012) Curr Top Med Chem 12(13):1456-67; and Bodera & Stankiewicz (2011) Recent Pat Endocr Metab Immune Drug Discov 5(3):192-6, the disclosures of which are incorporated herein by reference.

In certain embodiments, a TNF-α inhibitory agent useful in the methods presented herein is etanercept. Etanercept is described, for example, in U.S. Pat. No. 7,276,477 (hereby incorporated by reference in its entirety). Etanercept is a dimer of two polypeptides that each include a portion of the TNF-α receptor TNFR2 and a portion of a human IgG1. The amino acid sequence of the monomeric component of etanercept is disclosed in U.S. Pat. No. 7,276,477.

Other useful TNF-α inhibitory agents include but are not limited to, e.g., SSR150106 (Sanofi, Bridgewater, N.J.), TIMP-1, TIMP-2, adTIMP-1 (i.e., adenoviral delivered TIMP-1), adTIMP-2 (adenoviral delivered TIMP-2), prostaglandins; IL-10, which is known to block TNF-α production via interferon-γ-activated macrophages (Oswald et al., 1992, Proc. Natl. Acad. Sci. USA 89:8676-8680), TNFR-IgG (Ashkenazi et al., 1991, Proc. Natl. Acad. Sci. USA 88:10535-10539); the murine product TBP-1 (Serono/Yeda), the vaccine CytoTAb (Protherics), the peptide RDP-58 (SangStat), antisense molecule 104838 (ISIS), NPI-13021-31 (Nereus), SCIO-469 (Scios), TACE targeter (Immunix/AHP), CLX-120500 (Calyx), Thiazolopyrim (Dynavax), auranofin (Ridaura) (SmithKline Beecham Pharmaceuticals), quinacrine (mepacrine dichlorohydrate), tenidap (Enablex), Melanin (Large Scale Biological), and anti-p38 MAPK agents by Uriach, and those described in, e.g., U.S. Patent Publication No: 2009/0042875 A1 and PCT Publication No: WO 2002080892 A1, the disclosures of which are incorporated herein by reference.

In some instances, a TNF-α inhibitory agent may be a TNF-α interfering nucleic acid or a nucleic acid that interferes with the function or production of TNFα. TNF-α interfering nucleic acids useful in practicing the methods disclosed herein include, but are not limited to, e.g., dsRNA, siRNA, shRNA, ddRNAi, and the like.

TNF-α interfering nucleic acids useful in certain embodiments for practicing methods described herein may be generated using in vitro, in vivo, or synthetic production methods. For example, in vitro production may be achieved by cloning a TNF-α interfering nucleic acid construct in to an appropriate vector, e.g., a plasmid or phage DNA, used to generate the TNF-α interfering nucleic acid, and the TNF-α interfering nucleic acid is generated through the use of an in vitro transcription reaction. Any convenient method for in vitro transcription may find use in generating a TNF-α interfering nucleic acid of the subject disclosure including, but not limited to, an in vitro transcription kit or a dsRNA synthesis kit. Non-limiting examples of commercially available in vitro transcription kits and dsRNA synthesis kits include MEGAscript® RNAi Kits (Life Technologies, Grand Island, N.Y.), Replicator RNAi Kits (Thermo Scientific®, a division of Fisher Scientific®, Pittsburgh, Pa.), T7 RiboMAX™ (Promega Corporation, Madison, Wis.), MAXIscript® (Life Technologies, Grand Island, N.Y.), T7 High Yield RNA Synthesis Kit (New England Biolabs, Ipswich, Mass.), SP6/T7 Transcription Kit (Roche Applied Science, Indianapolis, Ind.), and the like.

In vivo production of TNF-α interfering nucleic acids for use in certain embodiments of the methods described herein include but are not limited to methods of transforming a TNF-α interfering nucleic acid producing construct (e.g., an expression vector comprising a nucleotide sequence encoding a TNF-α interfering nucleic acid) into an organism, e.g., a phage, a virus, a prokaryote, a eukaryote, a bacterium, a yeast, a cell of a cell culture system, a cell of a mammalian cell culture system, a plant, a cell of a plant cell culture system, and the like, for the purpose of generating a TNF-α interfering nucleic acid in vivo. Methods for production of TNF-α interfering nucleic acid in vivo, e.g., by introducing a dsRNA construct or a shRNA construct into a living cell by transformation with dsRNA constructs, are well known in the art, see, e.g., Timmons et al. (2001) Gene, 263:103-112; Newmark et al. (2003) Proc Natl Acad Sci USA, 100 Supp 1:11861-5; Reddien et al. (2005) Developmental Cell, 8:635-649; U.S. Pat. No. 6,506,559; and U.S. Pat. No. 7,282,564, the disclosures of which are incorporated herein by reference. Non-limiting examples of commercially available systems and materials for shRNA production include Knockout™ Inducible RNAi Systems (Clontech, Mountain View, Calif.), psiRNA™ Vectors (InvivoGen, San Diego, Calif.), MISSION® siRNA and shRNA systems (Sigma-Aldrich Co., St. Louis, Mo.), and the like.

In certain embodiments, a TNF-α interfering nucleic acid may be introduced into an organism through the use of a virus vector, e.g., a lentivirus vector. Such methods for introducing interfering nucleic acids using virus vectors and lentivirus vectors are well-known in the art. For example, in some cases, an expression vector including a nucleotide sequence encoding a TNF-α interfering nucleic acid is a virus-based vector, e.g., a lentivirus vector, an adenovirus vector, an adeno-associated virus vector, etc. In some cases, an expression vector including a nucleotide sequence encoding a TNF-α interfering nucleic acid includes a promoter operably linked to the nucleotide sequence encoding the TNF-α interfering nucleic acid. Suitable promoters include constitutive promoters and inducible promoters.

Synthetic production of TNF-α interfering nucleic acids for use in certain embodiments of the methods described herein include but are not limited to methods of synthetic siRNA production. In some embodiments, siRNA is produced by methods not requiring the production of dsRNA, e.g., chemical synthesis or de novo synthesis or direct synthesis. Chemically synthesized siRNA may be synthesized on a custom basis or may be synthesized on a non-custom or stock or pre-designed basis. Custom designed siRNA are routinely available from various manufactures (e.g., Ambion®, a division of Life Technologies®, Grand Island, N.Y.; Thermo Scientific®, a division of Fisher Scientific®, Pittsburgh, Pa.; Sigma-Aldrich®, St. Louis, Mo.; Qiagen®, Hilden, Germany; etc.).

Methods for design and production of siRNAs to a TNF-α target are known in the art, and their application to TNF-α inhibition for the purposes disclosed herein will be readily apparent to the ordinarily skilled artisan, as are methods of production of siRNAs having modifications (e.g., chemical modifications) to provide for, e.g., enhanced stability, bioavailability, and other properties to enhance use as therapeutics. In addition, methods for formulation and delivery of siRNAs to a subject are also well known in the art. See, e.g., US 2005/0282188; US 2005/0239731; US 2005/0234232; US 2005/0176018; US 2005/0059817; US 2005/0020525; US 2004/0192626; US 2003/0073640; US 2002/0150936; US 2002/0142980; and US2002/0120129, each of which are incorporated herein by reference. In vivo and in vitro methods for RNAi targeted at TNF-α are described in, e.g., Salako et al. (2011) Mol Ther 19(3):490-9, Wilson et al. (2010) Nat Mater 9(11):923-8, Jakobsen et al. (2009) Molecular Therapy 17(10):1743-1753, Qin et al. (2011) Artificial Organs 35(7):706-714, the disclosures of which are incorporated herein by reference.

### Methods of Measuring TNF-α and TNF-α Signaling

According to certain embodiments of the present disclosure, TNF-α levels or levels of TNF-α signaling may be assessed, e.g., for a sample obtained from a subject. Any convenient method of measuring TNF-α levels or levels of TNF-α signaling may be employed in making such assessments. In some instances, TNF-α levels or levels of TNF-α signaling may be assessed, either directly or indirectly, by measuring the level of TNF-α protein or the levels of one or more TNF-α pathway proteins present in a sample. For example, in some instances, TNF-α protein levels may be directly measured using any convenient assay for direct measurement of protein levels. In some instances, TNF-α levels may be measured indirectly, e.g., through the measurement of the levels of one or more TNF-α pathway member. For example, the TNF-α level may be assessed through measurement of the protein levels of one or more TNF-α pathway members that are known to increase when TNF-α signaling is active. In other instances, the TNF-α level may be assessed through measurement of the protein levels of one or more TNF-α pathway members that are known to decrease when TNF-α signaling is active.

Methods of measuring protein levels that find use in assaying the protein levels of TNF-α and TNF-α pathway members include but are not limited to Western blot, immunohistochemistry, flow cytometry, enzyme-linked immunosorbent assay (ELISA) based methods, mass spectrometry, immunochromatography, high performance liquid chromatography (HPLC), and the like. Such methods for both experimental and clinical use are well known in the art.

In certain instances, TNF-α levels may be assessed through the measurement of gene expression of one or more TNF-α pathway members known to be affected by TNF-α signaling. For example, in some instances, the gene expression of a TNF-α pathway member known to be upregulated upon activation of TNF-α signaling may be measured to assess TNF-α levels. In other instances, the gene expression of a TNF-α pathway member known to be down-regulated upon activation of TNF-α signaling may be measured to assess TNF-α levels. In some instances, the gene expression of a non-TNF-α pathway gene known to be co-expressed with TNF-α or a TNF-α pathway member upregulated or down-regulated upon TNF-α signaling may be measured to assess TNF-α levels.

Methods of measuring gene expression levels that find use in assaying the levels of TNF-α pathway members and non-TNF-α pathway genes include but are not limited to northern blot, quantitative reverse transcription-polymerase chain reaction (RT-PCR), in situ hybridization, reporter gene assays, quantitative sequencing (e.g., digital sequencing methods), microarrays, and the like. Such methods for both experimental and clinical use are well known in the art.

In certain instances, TNF-α levels may be assessed through the measurement of cellular expression of TNF-α or one or more TNF-α pathway members. As used herein “cellular expression” may refer to gene expression, e.g., mRNA expression, within a cell or protein expression within or on the surface of a cell. In some instances, cellular expression of TNF-α or one or more TNF-α pathway members may be measured in terms of the relative or absolute expression level, e.g., the amount of mRNA or protein, expressed by a particular cell or a population of cells. In other instances, expression of TNF-α or one or more TNF-α pathway members may be measured in terms of the relative or absolute number of cells of a population of cells expressing TNF-α or one or more TNF-α pathway members, e.g., the number of cells or the fraction of cells within a cell population expressing TNF-α or one or more TNF-α pathway members above or below a particular threshold. Such thresholds may vary and in some instances may represent the background or baseline expression level of TNF-α or one or more TNF-α pathway members in a reference cell or cell population for which the level of TNF-α expression is known, including, e.g., control cells known to not express TNF-α or one or more TNF-α pathway members, control cells known to express at a low level, control cells known to express at an average level or control cells known to express at a high level.

Methods of measuring cellular expression of TNF-α or one or more TNF-α pathway members that find use in assaying the levels of TNF-α pathway members and non-TNF-α pathway genes include but are not limited to methods for assaying cellular gene expression, including those methods described herein for measuring gene expression, and methods for measuring cytoplasmic and cell surface protein expression. Methods for measuring cytoplasmic protein expression and methods for measuring cell surface protein expression, including methods for measuring TNF-α and TNF-α pathway members, are well known in the art.

### Methods of Treating Subjects

Methods of treating a hearing disorder associated with maladaptive neuroplasticity, reduction of inhibition, shift of excitation-to-inhibition balance, changes in central gain, and/or changes in neural sensitivity and methods of preventing a hearing disorder associated with maladaptive neuroplasticity, reduction of inhibition, shift of excitation-to-inhibition balance, changes in central gain, and/or changes in neural sensitivity generally include administering to a subject an effective amount of a TNF-α inhibitory agent that reduces a level of TNF-α or TNF-α signaling. An effective amount of an agent reduces the level of TNF-α or TNF-α signaling by at least about 10%, at least about 20%, at least about 30%, at least about 40%, at least about 50%, or more, when compared to a suitable control. An effective amount of an agent reduces the symptoms of the hearing disorder associated with maladaptive neuroplasticity, reduction of inhibition, shift of excitation-to-inhibition balance, changes in central gain, and/or changes in neural sensitivity by at least about 10%, at least about 20%, at least about 30%, at least about 40%, at least about 50%, or more, when compared to a suitable control.

In some embodiments, a single dose of a TNF-α inhibitory agent is administered. In other embodiments, multiple doses of a TNF-α inhibitory agent are administered. Where multiple doses are administered over a period of time, a TNF-α inhibitory agent is administered twice daily (qid), daily (qd), every other day (qod), every third day, three times per week (tiw), or twice per week (biw) over a period of time. For example, a TNF-α inhibitory agent is administered qid, qd, qod, tiw, or biw over a period of from one day to about 2 years or more. For example, a TNF-α inhibitory agent is administered at any of the aforementioned frequencies for one week, two weeks, one month, two months, six months, one year, or two years, or more, depending on various factors.

Subjects in need of treatment include those subjects having a hearing disorder associated with maladaptive neuroplasticity, reduction of inhibition, shift of excitation-to-inhibition balance, changes in central gain, and/or changes in neural sensitivity and those subjects at risk of developing a hearing disorder associated with maladaptive neuroplasticity, reduction of inhibition, shift of excitation-to-inhibition balance, changes in central gain, and/or changes in neural sensitivity; as such, the methods of treating a hearing disorder described herein may be palliative or preventive. Subjects in need of preventative treatments include but are not limited to those subjects that experience chronic or acute exposure to loud noise, constant noise, blasts, explosions, potential head trauma, potential neck trauma, potential ear trauma and the like. In other instances, subjects in need of preventative treatments include but are not limited to those subjects that have risk factors for developing a hearing disorder associated with maladaptive neuroplasticity, reduction of inhibition, shift of excitation-to-inhibition balance, changes in central gain, and/or changes in neural sensitivity including but not limited to head and neck trauma, hypo- or hyperthyroidism, Lyme disease, fibromyalgia, thoracic outlet syndrome, certain tumors, wax build-up, jaw misalignment, cardiovascular disease, ototoxicity, frequent ear infection, and taking certain medications, e.g., including but not limited to aspirin and aspirin containing products, salicylates and methylsalicylates, NSAIDS (e.g., diclofenac (Voltaren), etocolac (Lodine), fenprofen (Nalfon), ibuprofen (Motrin, Advil, Nuprin, etc.), indomethacin (Indocin), naproxen (Naprosyn, Anaprox, Aleve), piroxicam (Feldene), sulindac (Clinoril)), antibiotics (e.g., amikacin (Amakin), gentamycin (Garamycin), kanamycin (Kantrex), neomycin, netilmicin (Netromycin), streptomycin, tobramycin (Nebcin), erythromycin (EES, E-mycin, Ilosone, Eryc, Pediazole, Biaxin, Zithromax), vancomycin (Vancocin), minocycline (Minocin), polymixin B & amphotericin B (Antifungal preparations), capreomycin (Capestat)), diuretics (e.g., bendroflumethazide (Corzide), bumetadine (Bumex), chlor-thalidone (Tenoretic), ethacrynic acid (Edecrin), furosemide (Lasix)), chemotherapeutic agents (e.g., bleomycine (Blenoxane), bromocriptine (Parlodel), carboplatinum (Carboplatin), cisplatin (Platinol), methotrexate (Rheumatrex), nitrogen mustard (Mustargen), vinblastin (Velban), vincristine (Oncovin)), quinine (e.g., chloroquine phosphate (Aralen), quinacrine hydrochloride (Atabrine), quinine sulfate (Quinam)), misoprostol (Cytotec), hydrocodone (Lorcet, Vicodin).

According to aspects of the present invention, a method includes administering to a subject the TNF-α inhibitory agent after the subject has been exposed to a noise or blast. For example, a method according to aspects of the invention may include administering to the subject the TNF-α inhibitory agent within about 1, 2, 3, 4, 5, 6, 7, 8, 9, 10, 11, 12, 13, 14, 15, 16, 17, 18, 19, 20, 21, 22, 23, or 24 hours, within about 1, 2, 3, 4, 5, 6, 7, 8, 9, or 10 days, within about 1, 2, 3, 4, 5, 6, 7, or 8 weeks, or within about 1 month after the subject has been exposed to a noise or blast (e.g., a noise or blast associated with an increased risk of a hearing disorder associated with maladaptive neuroplasticity, reduction of inhibition, shift of excitation-to-inhibition balance, changes in central gain, and/or changes in neural sensitivity).

In some instances, a subject in need of prevention and/or treatment of a hearing disorder associated with maladaptive neuroplasticity, reduction of inhibition, shift of excitation-to-inhibition balance, changes in central gain, and/or changes in neural sensitivity may be taking certain medications that can cause a hearing disorder associated with maladaptive neuroplasticity, reduction of inhibition, shift of excitation-to-inhibition balance, changes in central gain, and/or changes in neural sensitivity or exposed to certain chemicals that can cause a hearing disorder associated with maladaptive neuroplasticity, reduction of inhibition, shift of excitation-to-inhibition balance, changes in central gain, and/or changes in neural sensitivity, such drugs and chemicals including, e.g., but not limited to vapors, solvents (e.g. cyclohexane, dichloromethane, hexane (gasoline), lindane (Kwell), methyl-chloride, methyl-n-butyl-ketone, perchlor-ethylene, Styrene, tetrachlor-ethane, toluol, trichloroethylene), antibiotics (e.g., minoglycosides (see previous section), amphotericin B, chloramphenicol (Chloromycetin), minocycline (Minocin), polymyxine B, sulfonamides (Septra, Bactrim), vancomycin (Vancocin)), anti-neoplastics (e.g., carboplatinum (Paraplatin), methotrexate (Rheumatrex), nitrogen mustard (Mustagen), vinblastin (Velban)), diuretics (e.g., furosemide (Lasix), hydrochlorthiazide (Hydrodiuril), methylchlorthizide (Enduron)), cardiac medications (e.g., celiprolol, flecainide (Tambocar), lidocaine, metoprolol (Lopressor), procainamide (Pronestyl), propranolol (Inderal), quinidine (Quinaglute, Quinidex)), psychopharmacologic agents (e.g., amitryptiline (Elavil), benzodiazepine class (e.g., alprazolam (Xanax), clorazepate (Tranxene), chlordiazepoxide (Librium), diazepam (Valium), flurazepam (Dalmane), lorazepam (Ativan), midazolam (Versed), oxazepam (Serax), prozepam (Centrax), quazepam (Doral), temazepam (Restoril), triazolam (Halcion), bupropion (Welbutrin), carbamzepine (Tegretol), diclofensine, doxepin (Sinequin), desiprimine (Norpramin), fluoxetin (Prozac), imipramine (Tofranil), lithium, melitracen, molindon (Moban), paroxetin, phenelzin (Nardil), protriptilin (Vivactil), trazodon (Desyrel), zimeldin), NSAIDS (see above), asprin, acematacine, benorilate, benoxaprofen, carprofen, diclofenac (Voltaren), diflunisal (Dolobid), fenoprofen (Nalfon), feprazon, ibuprofen (Motrin, Advil, Nuprin), indomethacin (Indocin), isoxicam, ketoprofen (Orudis), methyl salicylates (BenGay), naproxen (Naprosyn, Anaprox, Aleve), D-Penicilliamin, phenylbutazone (Butazolidine), piroxicam (Feldene), proglumetacin, proquazon, rofecoxib (Vioxx), salicylates, sulindac (Clinoril), tolmetin (Tolectin), zomepirac, glucocorticosteroids (e.g., prednisolone (Prednisone), ACTH (adrenocorticotrophic hormone) (Acthar)), anesthetics (e.g., bupivacain, tetracain, lidocaine (Novacaine)), antimalarials (e.g., chloroquine (Aralen), hydroxychloroquine (Plaquinil)), thalidomide (Thalomid), alcohol, arsenum, caffeine, lead, marijuana, nicotine, mercury, and auronofin (gold, Ridaura).

Subjects having a hearing disorder associated with maladaptive neuroplasticity, reduction of inhibition, shift of excitation-to-inhibition balance, changes in central gain, and/or changes in neural sensitivity and in need of treatment include but are not limited to those subjects that acquired a hearing disorder associated with maladaptive neuroplasticity, reduction of inhibition, shift of excitation-to-inhibition balance, changes in central gain, and/or changes in neural sensitivity through any of the above described risk factors. For example, subjects that may be treated according to the inventive methods may be those subjects having a hearing disorder associated with maladaptive neuroplasticity, reduction of inhibition, shift of excitation-to-inhibition balance, changes in central gain, and/or changes in neural sensitivity caused by noise exposure, blast exposure, aging, disease, drug or chemical exposure, and the like. In some instances, subjects in need of treatment according to the methods described herein may be those subjects whose hearing disorder associated with maladaptive neuroplasticity, reduction of inhibition, shift of excitation-to-inhibition balance, changes in central gain, and/or changes in neural sensitivity is not caused by drug or chemical exposure or not a drug-induced hearing disorder, including, e.g., a salicylate-induced hearing disorder associated with maladaptive neuroplasticity, reduction of inhibition, shift of excitation-to-inhibition balance, changes in central gain, and/or changes in neural sensitivity.

In addition to administration of a TNF-α inhibitory agent according to the methods described herein, subjects may also receive additional agents or therapies directed to treating a hearing disorder associated with maladaptive neuroplasticity, reduction of inhibition, shift of excitation-to-inhibition balance, changes in central gain, and/or changes in neural sensitivity in the form of combination therapy. Such additional agents and therapies include but are not limited to, e.g., conventional hearing disorder therapies and conventional hearing disorder drug therapies. Conventional hearing disorder treatments may vary but include and are not limited to, e.g., counseling, sensory masking, sensory/cognitive training, mineral supplements (e.g., magnesium or zinc), herbal supplements (e.g., ginkgo biloba), homeopathic remedies, B vitamin supplement, acupuncture, cranio-sacral therapy, magnetic therapy, hyperbaric oxygen, hypnosis, sound amplification (e.g., hearing aids), biofeedback, cochlear implants/electrical stimulation, cognitive behavioral therapy, mindfulness based stress reduction, sound therapy, TMJ treatment, transcranial magnetic stimulation (TMS) and repetitive transcranial stimulation (rTMS).

In some instances, the methods described herein may include combination therapy with conventional drugs for treating a hearing disorder including but not limited to acamprosate, caroverine, memantine, AM-101, neramexane, gacyclidine, alprazolam, diazepam, clonazepam, vigabatrin, tiagabine, lidocaine, potassium channel modulators, carbamazepine, sodium valproate/valproic acid, gabapentin, trimipramine, nortriptyline, paroxetine, trazodone, vestipitant/paroxetine combination therapy, sertraline, misoprostol, atorvastatin, nimodipine, furosemide, amino-oxyacetic acid, scopolamine, cyclandelate, sulpiride, vardenafil, ginkgo biloba, melatonin, and zinc supplements, the structures, functions, and treatment modalities of which are described, e.g., in Salvi et al. (2009) Drugs Future 34(5):381-400, the disclosure of which is incorporated herein by reference.

In some instances, the method of treatment may include combination therapy with one or more ion channel inhibitors or enhancers, including but not limited to Calcium (Ca²⁺) channel blockers, Chloride (Cl⁻) channel blockers, Potassium (K⁺) channel blockers, Sodium (Na⁺) channel blockers, Zinc (Zn²⁺)-activated channel antagonists, Calcium (Ca²⁺) channel activators, Chloride (Cl⁻) channel activators, Potassium (K⁺) channel activators, Sodium (Na⁺) channel activators, Zinc (Zn²⁺)-activated channel activators, and the like. In some instances, the method of treatment may include combination therapy with one or more modulator of ligand gated channels that affect ion influx or efflux including but not limited to, e.g., 5-HT3 receptor antagonists, AMPA receptor antagonists, Kainate receptor antagonists, nACh receptor antagonists, NMDA receptor antagonists, P2X receptor antagonists, and the like. In other instances, the method of treatment may include combination therapy with one or more enhancer of GABA signaling, including but not limited to theanine, acamprosate, methaqualone, muscimol, picamilon, progabide, tiagabine, baclofen, 1,4-Butanediol, GBL (γ-Butyrolactone), GHB (γ-Hydroxybutyric acid), GHV (γ-Hydroxyvaleric acid), GVL (γ-Valerolactone), lesogaberan, phenibut, (Z)-4-Amino-2-butenoic acid, (+)-cis-2-Aminomethylcyclopropane carboxylic acid, N4-Chloroacetylcytosine arabinoside, GABOB (γ-Amino-beta-hydroxybutyric acid), progabide, and the like. In other instances, the method of treatment may include combination therapy with one or more enhancer of glycine synapses, including but not limited to alanine, cycloserine, dimethylglycine, ethanol, glycine, hypotaurine, methylglycine (sarcosine), milacemide, serine, taurine, trimethylglycine (betaine), and the like. In other instances, the method of treatment may include combination therapy with one or more inhibitor of glutamate synapses, including but not limited to bicuculline, brucine, caffeine, picrotoxin, strychnine, tutin, and the like.

In some instances, a TNF-α inhibitory agent of the subject method may be administered directly, e.g., surgically or by injection, to within the blood brain barrier (BBB). In other instances, the TNF-α inhibitory agent may be formulated to cross the BBB thus making direct administration unnecessary. In certain circumstances, neither direct administration within the BBB nor functionalization of the TNF-α inhibitory agent to cross the BBB is necessary due to permeabilization of the BBB. Permeabilization of the BBB may result as a consequence of the specific condition or incidence from which a subject's hearing disorder is a result or may be purposefully caused as a means of administering the TNF-α inhibitory agent. In some instances, exposure to trauma, e.g., blast exposure, may permeabilize the BBB allowing delivery across the BBB of a TNF-α inhibitory agent that is not functionalized to cross the BBB nor is directly delivered within the BBB. Conditions where the BBB of a subject is permissive to delivery of a TNF-α inhibitory agent including TNF-α inhibitory agents that have not been functionalized to cross the BBB may be determined by the ordinary skilled medical practitioner upon observation of the subject.

In certain embodiments, inhibition of TNF-α or TNF-α signaling is achieved through the inhibition of one or more endogenous host genes or one or more regulatory elements of one or more endogenous host genes within the genome of the subject to be treated. For example, in some instances, an endogenous gene locus or allele of TNF-α is modified within the genome of the subject in a manner effective to inhibit TNF-α expression or TNF-α signaling. In other instances, an endogenous regulatory element is modified, e.g., inhibited, repressed, enhanced, or deleted, within the genome of the subject in a manner effective to inhibit TNF-α expression or TNF-α signaling. In accordance with such methods, a construct may be inserted into the genome of a subject in a site specific or in a non-specific manner as such methods are known in the art, including but not limited to, e.g., virus mediated integration, transposon mediated integration, homologous recombination, zinc-finger nuclease mediated integration, CRISPR mediated integration, and the like. Methods of introducing constructs for both transient and stable modification are well-known and described in, e.g., Strachan & Read (1999) Human Molecular Genetics. 2nd edition. New York: Wiley-Liss, the disclosure of which is incorporated herein by reference. Such modification of cells and/or organisms as is described herein may be performed in vivo, e.g., through the modification of the cells within their endogenous tissues within an organism, or ex vivo, e.g., through the in vitro modification of cells and the subsequent re-introduction of the cells into the organism.

In other instances, an endogenous gene locus or allele of TNF-α is unmodified and the genome of the subject is modified at in a manner effective to inhibit TNF-α expression or TNF-α signaling. For example, in some instances, inhibition of TNF-α, TNF-α expression or TNF-α signaling may be achieved through the transient, e.g., extrachromosomal, or stable, e.g., genomic integration, introduction of one or more constructs that expresses an inhibitor or repressor of TNF-α, TNF-α expression or TNF-α signaling including but not limited to a dominate negative TNF-α, dominate negative TNF-α pathway member, or other TNF-α inhibitory agent.

Genome modification of the subject may be transient, e.g., temporary, or non-transient, e.g., irreversible or stable, and may be systemic, e.g., affecting all or most cells of an organism, local, e.g., restricted to a particular area, tissue specific, e.g., restricted to a particular tissue of an organism, or cell specific, e.g., restricted to a particular cell type or a particular group of cell types. Methods for systemic, e.g., through the use of constitutively active promoters, and tissue or cell specific, e.g., through the use of tissue specific or cell specific promoters, are well known in the art. In some instances, the modified locus may be stably modified such that the modification lasts for the life of modified cell or the life of the modified subject. In some instances, the modified locus may be transiently modified such that the modification is reversible, e.g., through activation of a mechanism that reverses the modification, or the modification is self-reversing, e.g., through mechanisms induced by the modification itself or through cellular mechanisms that silence or reverse the modification. In some instances, irreversible modification may be achieved through the use of unidirectional modification. In other instances, reversible modification may be achieved through the use of bidirectional modification. Any convenient method for reversible gene modification or regulatory element modification may be utilized including but not limited to systems based on homologous recombination including, e.g., Cre/Lox, Flp-Frt, R-RS, and the like, which are described in, e.g., Wang et al. (2011) Plant Cell Rep 30(3): 267-285, the disclosure of which is incorporated herein by reference.

In other instances, transient gene therapy used in the methods described herein is achieved through the use of an exogenous gene or other expressible TNF-α inhibitory agent that does not integrate into the genome of the subject. Examples of such methods of transient exogenous gene or expressible TNF-α inhibitory agent transfer will vary and generally involve introduction of the gene or agent on an extrachromosomal construct or fragment thereof including not limited to, e.g., an artificial chromosome, a BAC, a YAC, a plasmid, plasmid DNA, viral DNA, viral RNA, a viral genome, minicircle DNA, dsRNA, siRNA, shRNA, an interfering oligonucleotide, and the like. In some instances, the introduced transient exogenous gene or expressible TNF-α inhibitory agent may be inducible, e.g., through the use of an inducible expression system including but not limited to expression systems utilizing an inducible promoter and/or a transactivator.

In some instances, the methods described herein include assessing and/or diagnosing a hearing disorder associated with maladaptive neuroplasticity, reduction of inhibition, shift of excitation-to-inhibition balance, changes in central gain, and/or changes in neural sensitivity in the subject. Any convenient hearing disorder assessment may find use in the methods described herein. Such hearing disorder assessments include but are limited to audiometric evaluation, audiometry, psychoacoustic evaluation, otoscopy, tympanometry, otoacoustic emission testing, tinnitus pitch matching, tinnitus pitch masking, loudness sensitivity level testing, residual inhibition testing, and the like. In some instances, clinical evaluation of a hearing disorder may include physical assessments and/or imaging including but not limited to otoscope evaluation, MRI, CT scan, and the like. Methods for diagnosing and evaluating a hearing disorder include, but are not limited to, a tuning fork test and an Audiometer test, in which an audiologist presents a range of sounds of various tones or words and asks the patient to indicate whether a sound can be heard. In both humans patients and animals, acoustically (electrically, for cochlear implantees, EABR) evoked auditory brainstem responses (ABR) refer to evoked potentials generated by brief clicks or tone pips transmitted from an acoustic transducer in the form of an insert earphone or headphone (or electrical pulses for EABR in cochlear implant patients). The elicited waveform response is measured by surface electrodes typically placed at the vertex of the scalp and ear lobes. The amplitude (microvoltage) of the signal is averaged and charted against time. In animals, cellular biology techniques such as histology are used to reveal whether hair cells, their supporting cells and other microstructures in the inner ears are affected by injury. At a molecular level, gene expression and signaling pathways are also assessed to determine hearing-related changes. Methods for diagnosing and evaluating tinnitus are described in, e.g., Crummer et al. (2004) Am Earn Physician 69(1):120-126 and Salvi et al. (2009) Drugs Future 34(5):381-400, the disclosures of which are incorporated herein by reference.

In some instances, hearing disorder assessments are performed before, during or following treatment. In certain embodiments, hearing disorder treatment may be modified, for example, the dose or dose frequency of an administered TNF-α inhibitory agent may be adjusted according to the results of a hearing disorder assessment. In some instances, administration of a TNF-α inhibitory agent is begun or stopped based on the results of a hearing disorder assessment.

### Pharmaceutical Compositions and Formulations

As mentioned above, an effective amount of the TNF-α inhibitory agent is administered to the subject, where “effective amount” means a dosage sufficient to produce a desired result. In some embodiments, the desired result is at least a reduction in a hearing disorder associated with maladaptive neuroplasticity, reduction of inhibition, shift of excitation-to-inhibition balance, changes in central gain, and/or changes in neural sensitivity of a subject as compared to a control. In some embodiments, the desired result is a decrease in TNF-α activity or TNF-α signaling as compared to a control.

Typically, the compositions of the instant invention will contain from less than 1% to about 95% of the active ingredient, preferably about 10% to about 50%. Generally, between about 100 mg and 500 mg will be administered to a child and between about 500 mg and 5 grams will be administered to an adult.

In some instances, the dosage of a TNF-α inhibitory agent to be used in a human subject may be based on an effective dose of the agent as determined through pre-clinical testing, e.g., animal trials. For example, in some instances, a dosage of a TNF-α inhibitory agent, e.g., a dosage of thalidomide, found to be an effective dose in animal studies, e.g., rodent studies including mouse studies or rat studies, may be converted to a human equivalent dose (HED) for use in humans. Conversion of an animal dose to a HED may, in some instances, be performed using a conversion table (see, e.g., Table 1 below) and/or an algorithm provided by the U.S. Department of Health and Human Services, Food and Drug Administration, Center for Drug Evaluation and Research (CDER) in, e.g., Guidance for Industry: Estimating the Maximum Safe Starting Dose in Initial Clinical Trials for Therapeutics in Adult Healthy Volunteers (2005) Food and Drug Administration, 5600 Fishers Lane, Rockville, Md. 20857; (available at www(dot)fda(dot)gov/cder/guidance/index(dot)htm, the disclosure of which is incorporated herein by reference).

TABLE 1
Conversion of Animal Doses to Human Equivalent
Doses Based on Body Surface Area

| | | To Convert Animal | |
|---|---|---|---|
| | To Convert Animal | Dose in mg/kg to | |
| | Dose in mg/kg to | HED^{a} in mg/kg, Either: | |
| | Dose in mg/m², | Divide Animal | Multiply Animal |
| Species | Multiply by kₘ | Dose By | Dose By |
| Human | 37 | — | — |
| Child (20 kg)^{b} | 25 | — | — |
| Mouse | 3 | 12.3 | 0.08 |
| Hamster | 5 | 7.4 | 0.13 |
| Rat | 6 | 6.2 | 0.16 |
| Ferret | 7 | 5.3 | 0.19 |
| Guinea pig | 8 | 4.6 | 0.22 |
| Rabbit | 12 | 3.1 | 0.32 |
| Dog | 20 | 1.8 | 0.54 |
| Primates: | | | |
| Monkeys^{c} | 12 | 3.1 | 0.32 |
| Marmoset | 6 | 6.2 | 0.16 |
| Squirrel | 7 | 5.3 | 0.19 |
| monkey | | | |
| Baboon | 20 | 1.8 | 0.54 |
| Micro-pig | 27 | 1.4 | 0.73 |
| Mini-pig | 35 | 1.1 | 0.95 |

^{a}Assumes 60 kg human. For species not listed or for weights outside the standard ranges, HED can be calculated from the following formula: HED = animal dose in mg/kg × (animal weight in kg/human weight in kg)0.33.
^{b}This km value is provided for reference only since healthy children will rarely be volunteers for phase 1 trials.
^{c}For example, cynomolgus, rhesus, and stumptail.

Administration may be performed by injection, nanoinjection, injection array, injection to a localized area, infusion, transfusion, oral administration, topical administration, transdermal administration, transmucosal administration, inhalation, acoustic assisted administration, and the like. The frequency of administration will be determined by the care given based on patient responsiveness. Other effective dosages can be readily determined by one of ordinary skill in the art through routine trials establishing dose response curves.

In various methods of the invention, the active agent(s) may be administered to the subject using any convenient means capable of resulting in the desired inhibition of TNF-α activity or TNF-α signaling. Thus, the agent can be incorporated into a variety of formulations for therapeutic administration. More particularly, the agents of the present invention can be formulated into pharmaceutical compositions by combination with appropriate, pharmaceutically acceptable carriers or diluents, and the agents may be formulated into preparations in solid, semi-solid, liquid or gaseous forms, such as tablets, capsules, powders, granules, ointments, solutions, suppositories, injections, inhalants and aerosols.

As such, administration of the agents can be achieved in various ways, including oral, buccal, rectal, parenteral, intraperitoneal, intradermal, transdermal, intratracheal, etc., administration.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and/or flavoring agents.

Suitable excipient vehicles are, for example, water, saline, dextrose, glycerol, ethanol, or the like, and combinations thereof. In addition, if desired, the vehicle may contain minor amounts of auxiliary substances such as wetting or emulsifying agents or pH buffering agents. Actual methods of preparing such dosage forms are known, or will be apparent, to those skilled in the art. See, e.g., Remington's Pharmaceutical Sciences, Mack Publishing Company, Easton, Pa., 17th edition, 1985. The composition or formulation to be administered will, in any event, contain a quantity of the TNF-α inhibitory agent adequate to achieve the desired state in the subject being treated.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The agents can be utilized in an aerosol formulation to be administered via inhalation or for direct administration in aerosol form to the affected area. The compounds of the present invention can be formulated into pressurized acceptable propellants such as dichlorodifluoromethane, propane, nitrogen and the like.

In some instances, the active agent is configured to cross the blood brain barrier. For example, the active agent may be conjugated to a moiety that confers upon the active agent the ability to cross the blood brain barrier. Such a configuration allows for the targeting of the active agent to tissues within the blood brain barrier. In some embodiments, the subject moiety may be a peptide, e.g., vasoactive intestinal peptide analog (VIPa) or a cell-penetrating peptide. Suitable peptides that facilitate crossing of the blood brain barrier include, but are not limited to positively charged peptides with amphipathic characteristics, such as MAP, pAntp, Transportan, SBP, FBP, TAT₄₈₋₆₀, SynB1, SynB3 and the like.

In some embodiments, the subject moiety may be a polymer. Suitable polymers that facilitate crossing of the blood brain barrier include, but are not limited to, surfactants such as polysorbate (e.g., Tween® 20, 40, 60 and 80); poloxamers such as Pluronic® F 68; and the like. In some embodiments, an active agent is conjugated to a polysorbate such as, e.g., Tween® 80 (which is Polyoxyethylene-80-sorbitan monooleate), Tween® 40 (which is Polyoxyethylene sorbitan monopalmitate); Tween® 60 (which is Polyoxyethylene sorbitan monostearate); Tween® (which is Polyoxyethylene-20-sorbitan monolaurate); polyoxyethylene 20 sorbitan monopalmitate; polyoxyethylene 20 sorbitan monostearate; polyoxyethylene 20 sorbitan monooleate; etc. Also suitable for use are water soluble polymers, including, e.g., polyether, for example, polyalkylene oxides such as polyethylene glycol (“PEG”), polyethylene oxide (“PEO”), polyethylene oxide-co-polypropylene oxide (“PPO”), co-polyethylene oxide block or random copolymers, and polyvinyl alcohol (“PVA”); poly(vinyl pyrrolidinone) (“PVP”); poly(amino acids); dextran, and proteins such as albumin. Block co-polymers are suitable for use, e.g., a polyethylene oxide-polypropylene oxide-polyethylene-oxide (PEO-PPO-PEO) triblock co-polymer (e.g., Pluronic® F68); and the like; see, e.g., U.S. Pat. No. 6,923,986. Other methods for crossing the blood brain barrier are discussed in various publications, including, e.g., Chen & Liu (2012) Advanced Drug Delivery Reviews 64:640-665.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may include the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the subject.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Where the agent is a polypeptide, polynucleotide, analog or mimetic thereof, e.g. antisense composition, it may be introduced into tissues or host cells by any number of routes, including viral infection, microinjection, or fusion of vesicles. Jet injection may also be used for intramuscular administration, as described by Furth et au (1992), Anal Biochem 205:365-368. The DNA may be coated onto gold microparticles, and delivered intradermally by a particle bombardment device, or “gene gun” as described in the literature (see, for example, Tang et al. (1992), Nature 356:152-154), where gold microprojectiles are coated with the therapeutic DNA and then bombarded into skin cells.

Those of skill will readily appreciate that dose levels can vary as a function of the specific compound, the severity of the symptoms and the susceptibility of the subject to side effects. Preferred dosages for a given compound are readily determinable by those of skill in the art by a variety of means.

By treatment is meant at least an amelioration of the symptoms associated with the pathological condition afflicting the subject, where amelioration is used in a broad sense to refer to at least a reduction in the magnitude of a parameter, e.g. symptom, associated with the pathological condition being treated, such as hearing deficit. As such, treatment also includes situations where the pathological condition, or at least symptoms associated therewith, are completely inhibited, e.g. prevented from happening, or stopped, e.g. terminated, such that the subject no longer suffers from the pathological condition, or at least the symptoms that characterize the pathological condition.

Kits with unit doses of the active agent, usually in oral or injectable doses, are provided. In such kits, in addition to the containers containing the unit doses will be an informational package insert describing the use and attendant benefits of the drugs in treating pathological condition of interest. Preferred compounds and unit doses are those described herein above.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Methods

The following methods relate to the results provided in the Examples as presented herein and elsewhere where indicated.

### Noise Exposure and Auditory Brainstem Response (ABR)

All experimental procedures were reviewed and approved by UC Berkeley Animal Care and Use Committee. TNF-α knockout (KO) mice and corresponding C75Bl/6 wild-type (WT) mice were originally purchased from the Jackson Laboratory and were bred in a UC Berkeley animal facility. Animals were anesthetized with ketamine (100 mg/kg, IP) and xylazine (10 mg/kg, IP) and maintained at 36.5° C. with a homeothermic heating pad (Harvard Apparatus). Unilateral noise-induced hearing loss (NIHL) experiments were performed in a sound attenuation chamber by playing a continuous pure tone of 8 kHz at 112 dB SPL through a calibrated custom-made piezo earphone speaker to the left ear of the mouse for 2 hours, while the right ear was protected with sound attenuating clay. The sound level was calibrated with a Bruel and Kjaer 4135 condenser microphone (Nærum, Denmark) before and after the NIHL.

Hearing thresholds were assessed using auditory brainstem responses (ABR). ABR signals were recorded using the BioSigRP software on a TDT RX5 Sys3 recording rig. Tone pips (3-ms full-cycle sine waves at 4, 8, 16 and 32 kHz at 5-dB intensity steps from 0 to 70 dB) were delivered to the ears at a rate of 19 times per second through a calibrated TDT earphone, and 500 recordings were averaged to generate each ABR trace. ABR signals were recorded with three electrodes subcutaneously inserted behind the ear ipsilateral to the speaker, at the vertex of the head, and at the back of the body near the tail. The sound level that activated a minimal discernable response was defined as the auditory threshold for the particular frequency for each ear.

Behavioral Test of Tinnitus with a Gap Detection Task

Tinnitus was assessed using the gap detection paradigm. The gap detection task measures the acoustic startle response elicited by a brief white noise pulse and its suppression by a preceding silent gap embedded in a background sound. This paradigm has recently been confirmed to detect tinnitus in human subjects. Mice were placed in a small box, which rested atop a piezoelectric sensor within a sound attenuation chamber. Sounds were played through an open field speaker (FOSTEX FT17H) fixed above the small box. Each trial began with a carrier pure tone (frequency pseudo-randomly selected from 5, 7, 10, 14, 20, 28, or 45 kHz, all at 75 dB SPL), played for 10-20 s. In uncued trials, the carrier tone was followed by a startle stimulus—a 50 ms white noise burst at 102 dB SPL. In cued trials, the startle stimulus was preceded by a 50 ms silence, 100 ms prior to its onset. In each testing session, the animal performed a total of 500 trials (50% cued and 50% uncued). After each session, the startle response ratio was calculated, which is defined as the average startle amplitude to the cued trials divided by the average amplitude of the uncued trials. The startle response ratio signifies a silent-gap induced reduction of the startle response. For example, a startle response ratio of 0.6 indicates a 40% reduction of the startle amplitude for the cued trials. A startle response ratio of 1 suggests that the animal failed to detect the silent gap.

To assess an animal's ability to perform an auditory task, separate from its ability to detect a silent gap, the pre-pulse inhibition (PPI) task was administered to a subset of the mice before and after NIHL. The physical setup for the PPI task was identical to that of the gap detection. However, the trial structure differed in that carrier tone was absent and a white noise burst was cued by a 50-ms pure tone pulse (frequency pseudo-randomly selected from 5, 7, 10, 14, 20, 28, or 45 kHz, all at 75 dB SPL). In short, the PPI task tests an animal's ability to detect a pure tone pulse in silence, while the gap detection task measures an animal's ability to detect a silent gap in a continuous pure tone.

Mice were first acclimated to the testing chamber and trained until the behavior stabilized across two days. On average, 1000 trials were given prior to the first test session. Individual animals' performance was compared before and after the experimental manipulation. An increase of gap ratio accompanied by i) normal ABR for the intact ear and ii) normal PPI behavior were assumed to indicate tinnitus. Because both the gap detection task and the PPI task require normal hearing and hearing sensitivity above 32 kHz was highly variable across animals, only trials with carrier frequencies between 5 and 20 kHz were included in the final analysis.

### Injection of Recombinant TNF-α in the Auditory Cortex

Mice were anaesthetized with ketamine (100 mg/kg IP) and xylazine (10 mg/kg IP). Injection was done stereotactically to the right auditory cortex. A burr hole was made on the temporal ridge 1.75 mm anterior to the transverse suture. A pulled glass micropipette filled with recombinant mouse TNF-α (66.6 ng/μl in 1% mouse albumin fraction V) or 1% mouse albumin fraction V solution was lowered to 500 am below the pial surface and 1.5 μl solution was injected at 100 nl/min by pressure injection (Stoelting Quintessential Injector, Wood Dale, Ill., USA). The micropipette was then retracted 250 μm and an additional 1.5 μl of virus solution was injected. To minimize leaking, the micropipette was left in place for 8 min after each injection. In total, the experimental group received a dose of 200 ng of recombinant TNF-α to right auditory cortex. After injection, the skin was sutured and the animals were returned to their home cages after regaining movement. For postoperative pain management, animals received subcutaneous injection of buprenorphine (0.05 mg/kg, SQ) and meloxicam (2 mg/kg, SQ).

Measuring mRNA Levels with RT-PCR

After behavioral testing, animals were euthanized with isoflurane. Brain tissue was collected from the right and left auditory cortices based on anatomical landmarks by an experienced experimenter. A coronal slice of approximately 1 mm thickness (estimated stereotaxic coordinates: −2 mm to −3 mm bregma) was made using the dorsal-ventral extent of the hippocampus as landmarks. The auditory cortex at each side was then hemisected and isolated by making two orthogonal cuts to the cortical surface at 1 mm and 2 mm dorsal to the lingual gyms. Subcortical structures were removed and two 1 mm cubes of cortical tissue, one from each side, were collected. These samples presumably included the primary auditory cortex and possibly other fields of the auditory cortex.

Reverse transcription polymerase chain reaction (RT-PCR) was conducted by an experimenter who was blind to the experimental conditions. Total RNA samples were prepared from the tissue with RNA Wiz (Ambion) according to the manufacturer's instructions. The total RNA obtained (˜3 μg) was reverse-transcribed using a first-strand cDNA synthesis kit (BD Biosciences, Palo Alto, Calif.). The PCR mixture (50 μl) contained 10×Taq buffer, 0.3 U Taq polymerase (Perkin-Elmer), 2.5 μM of dNTPs, 5 pmol of each set of primers, and 50 ng of cDNA from the auditory cortex as template. PCR reactions were performed under the following cycling conditions: an initial denaturation at 94° C. for 5 min followed by 25-40 cycles of denaturation at 94° C. for 30 s, annealing at 63° C. for 30 s, and elongation at 72° C. for 1 min with a final elongation step at 72° C. for 10 min. A 10 μl sample of each PCR reaction was removed after 25 cycles, while the remaining mixture underwent 5 more cycles of amplification. The extent of amplification was chosen empirically to avoid saturation of the amplified bands. The 18S rRNA gene was used as an internal standard (QuantumRNA, Ambion). To quantify PCR products, each sample was run in a 1.5% agarose gel and stained with ethidium bromide. Band intensity was measured with an Alphaimager (Alpha Innotech Corp.) using the Alphaease (v3.3b) program.

### Electrophysiological Recording Procedure

The primary auditory cortex (AI) in naïve and sound-exposed KO and WT mice was mapped. Mice were anesthetized with ketamine (100 mg/kg, IP) and xylazine (10 mg/kg, IP), and placed on a homeothermic heating pad at 36.5° C. (Harvard Apparatus) in a sound attenuation chamber. The head was secured with a custom head-holder that left the ears unobstructed. The right auditory cortex was exposed and kept under a layer of silicone oil to prevent desiccation. Neural responses were recorded using tungsten microelectrodes (FHC) at a depth of 380-420 μm below the cortical surface, presumably from the thalamorecipient layer. Responses to 25-ms tone pips of 41 frequencies (4 to 75 kHz, 0.1 octave spacing) and eight sound pressure levels (10-80 dB, 10-dB steps) were recorded to reconstruct the frequency-intensity receptive field. A TDT coupler model electrostatic speaker was used to present all acoustic stimuli and each frequency×intensity combination was repeated 3 times. Both ears were stimulated in isolation to record contralateral and ipsilateral receptive fields at each recorded site.

Multi-unit activity was evenly sampled from the primary auditory cortex (AI), which could be identified by its tonotopic orientation—higher frequencies are represented more rostrally and slightly more dorsally—and location relative to cranial anatomical landmarks—AI was found consistently underneath the caudal half of the temporal-parietal bone suture. The border of AI was defined by unresponsive sites or sites whose characteristic frequencies (CFs) were incongruent with the AI tontopic gradient. Because KOs tended to have incomplete representations of low and high frequencies, those representations near the rostral and caudal ends of AI in both WTs and KOs were carefully searched for while maintaining the same sampling density. After monaural NIHL, cortical responses to the contralateral ear became weaker, and therefore AI was defined by the ipsilateral ear responses or the location relative to anatomical landmarks.

### Data Analysis

The receptive fields and response properties were computed using custom-made programs. First, the peri-stimulus time histogram (PSTH) was generated from responses to all 1032 (43 frequencies×8 intensities×3 repetitions) tone pips, with 1-ms bin size. The mean firing rate was calculated for each bin and smoothed with a 5-point mean filter. The multiunit firing rate in the 50-ms window prior to stimulus onset was taken as the mean spontaneous firing rate. Peak latency was defined as the time to the peak PSTH response between 7 and 50 ms after the stimulus onset. The response window was defined as the period encompassing the PSTH peak, in which the mean firing rate in every bin was higher than baseline firing rate. The onset latency was defined at onset of the response window. The tone-evoked response was measured as the maximum firing rate within the response window. Spikes that occurred within the response window were counted to reconstruct the receptive field.

The frequency-intensity receptive field (RF) was determined using a smoothing and thresholding algorithm. The response magnitude was plotted in the frequency-intensity space, and smoothed with a 3×3 mean filter (see, e.g., Yang et al. Cereb Cortex (2014) 24(7):1956-65, the disclosure of which is incorporated herein by reference). It was then thresholded at 28% of the maximum value of the smoothed response magnitude. The largest contiguous response area was determined to be the receptive field. The raw responses in the suprathreshold area was defined as the isolated receptive field. RF size was computed as the number of responsive frequency-intensity pairs in the isolated receptive field. The threshold of the neuron was the lowest sound level that elicited responses in the isolated receptive field, and the characteristic frequency (CF) was defined as the frequency that elicited responses at the threshold intensity. Manual ratings were carried out by an experienced rater blind to experimental condition. The maximum RF response was the maximum number of spikes activated by a single frequency-intensity combination. The mean RF response was the mean number of spikes for all frequency-dB combinations within the receptive field. Since each frequency-intensity combination was repeated 3 times, the average of those 3 responses was taken. The receptive field size was the number of frequency-intensity combinations within the receptive field.

Receptive field and map properties were analyzed using a three-way ANOVA with factors of genotype (WT or KO), experience (naïve or NIHL), and stimulation side (left or right). The statistical significance of differences between pairs of treatment means was assessed using Tukey's HSD (honest significant difference) multiple comparisons test.

### Statistically Significant Differences

Throughout the figures, a single asterisk (*) refers to a p-value less than 0.05, a double asterisk (**) refers to a p-value less than 0.01, and a triple asterisk (***) refers to a p-value less than 0.001.

### Example 1: NIHL Causes Tinnitus in WT but not TNF-α KO Mice

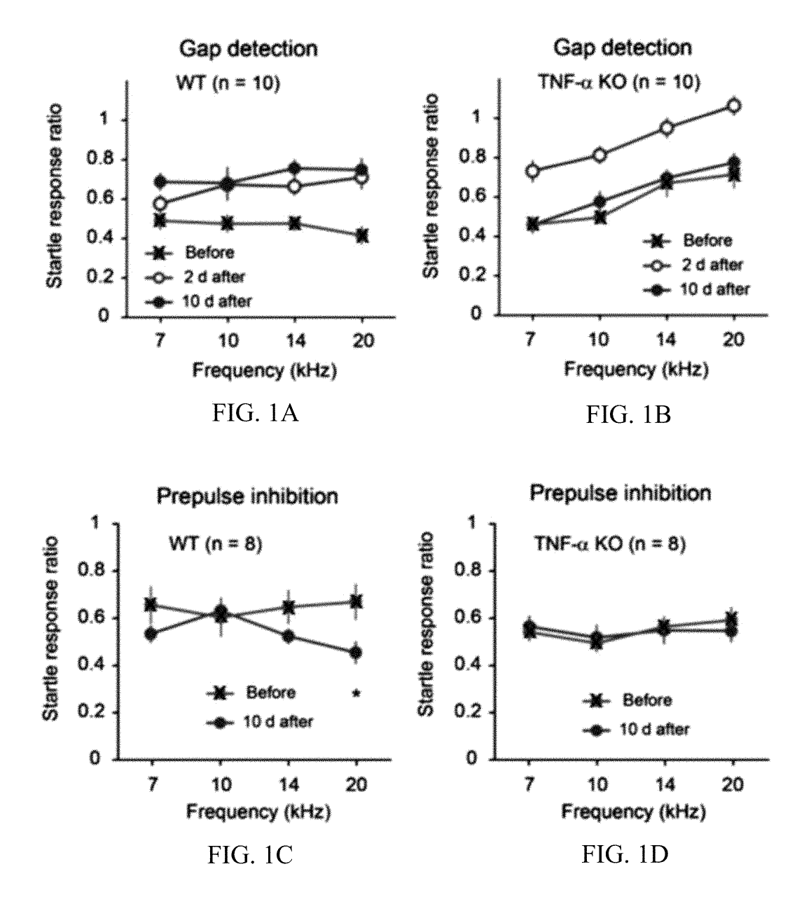

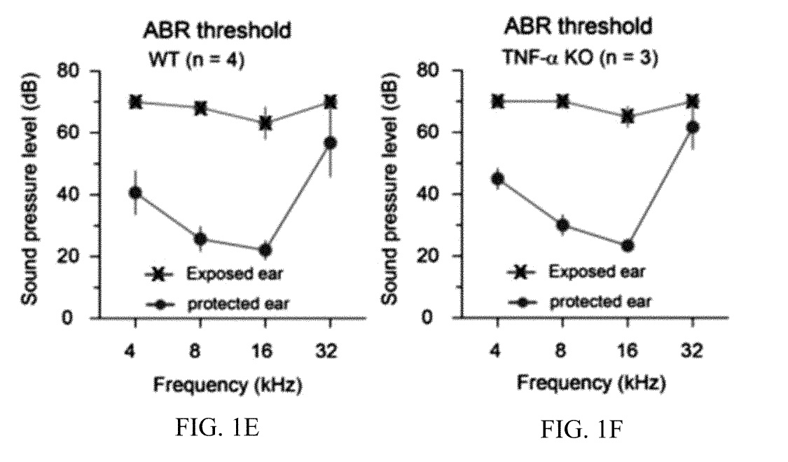

The left ear of anesthetized WT and KO mice were exposed to a 112-dB 8-kHz tone for 2 hours while the right ears of the mice were protected with sound attenuating clay. Behavioral evidence of NIHL-induced tinnitus was assessed by comparing gap detection performances before and after NIHL. Gap detection was impaired in both WT and KO mice 2 days after the sound exposure (FIGS. 1A-1B). However, by 10 days after sound exposure, gap detection performance of the KO mice had improved to the pre-exposure level (FIG. 1B). By contrast, gap detection performance of the WT mice remained impaired (FIG. 1A). A genotype-by-session 2-way ANOVA revealed significant effects for genotypes (F_{1,234}=15.37, p<0.0001), sessions (F_{2,216}=40.76, p<0.0001) and genotype-by-session interaction (F_{2,216}=17.96, p<0.0001), indicating that the effect of sound exposure on gap detection performance was different between WT and KO mice.

FIGS. 1A-1B demonstrate that TNF-α knockout mice do not develop chronic tinnitus following NIHL as seen in wild type mice. These results suggest that any therapeutic agent that decreases TNF-α activity (e.g., TNF-α inhibitors) will generally have a beneficial therapeutic effect in subjects with a hearing condition.

To determine whether the impaired gap detection in the WT mice was due to impairment in acoustic startle reflex, prepulse inhibition of startle reflex was examined in a subset of the animals before and 10 days after the sound exposure (FIG. 1C). A 2-way ANOVA indicates that there were no significant effects for WT and KO genotypes (F_{1,124}=2.44, p=0.12), sessions (F_{1,124}=3.87, p=0.052) or their interaction (F_{1,124}=3.44, p<0.066) (FIGS. 1C-1D). Sound exposure-induced ABR threshold shift was also evaluated (FIGS. 1E-1F). WT and KO mice showed similar amounts of threshold increase in the exposed ear compared to the protected ear (genotype-by-side-by-frequency 3-way ANOVA, genotype effect, F_{1,40}=1.80, p=0.19; genotype-by-side interaction, F_{1,40}=0.15, p=0.70; genotype-by-frequency interaction, F_{3,40} 0.11, p=0.96; 3-way interaction, F_{3,40}=0.15, p=0.93). The sound exposure procedure used in the present study does not cause threshold shift in the protected ear. These results demonstrate that WT, but not TNF-α KO mice, exhibit behavioral evidence of tinnitus 10 days after unilateral exposure to intense sound.

### Example 2: Binaural Plasticity Following Unilateral Hearing Loss is Impaired in TNF-α KO Mice

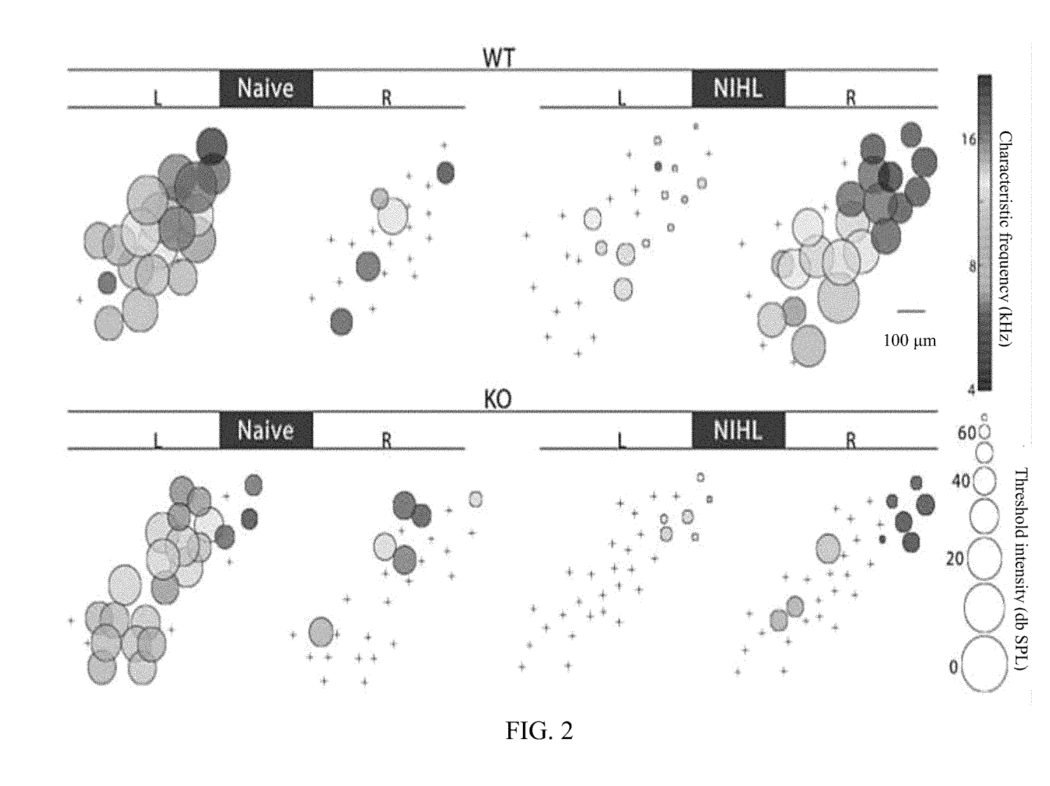

FIG. 2 provides example contralateral (L) and ipsilateral (R) maps for WT and KO in naïve and NIHL animals demonstrating that contralateral responses are nearly eliminated following NIHL in WT and KO, however only WT animals show strong augmentation of the ipsilateral map. Each circle represents the multi-unit recording from one site, with characteristic frequency and threshold intensity represented by color and radius, respectively. Unresponsive sites are marked by a +. In naïve WT and KO animals, contralateral maps in naïve animals have low thresholds and few non-responsive sites, while ipsilateral maps have few responsive sites. Contralateral responses are nearly eliminated following NIHL in WT and KO, however only WT animals show strong augmentation of the ipsilateral map.

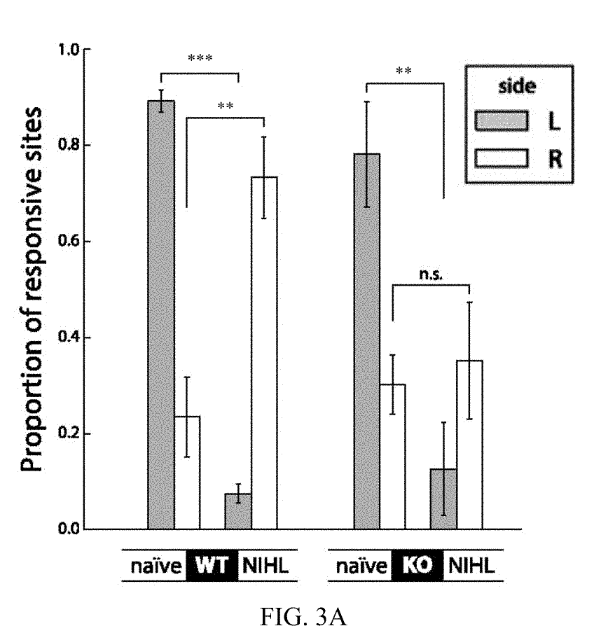

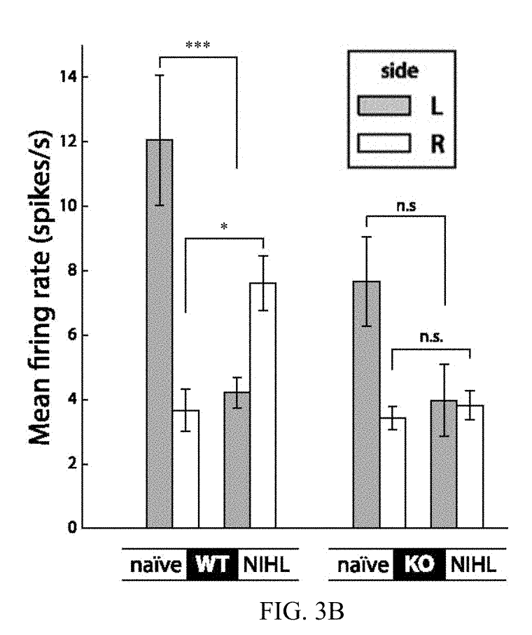

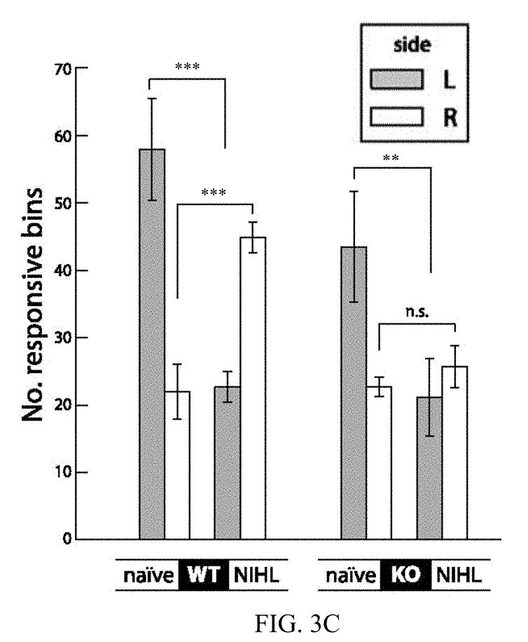

FIGS. 3A-3C demonstrate the absence of ipsilateral response development in KO post-NIHL. FIG. 3A shows the proportion of analyzed neurons responsive to ipsilateral and contralateral sound stimulations. FIG. 3B shows firing rate of neurons in response to sound stimulation. FIG. 3C shows the size of the receptive field in number of bins (frequency-intensity combinations).

To examine the electrophysiological changes in primary auditory cortex following unilateral NIHL, the lesioned and intact ear were independently stimulated while recording multi-unit activity from auditory cortex contralateral to the lesioned ear. Naïve WT and KO animals displayed strong, tonotopically-organized RFs in response to contralateral stimulation (FIG. 2). Unilateral NIHL led to a drastic reduction in the proportion of units responsive to the lesioned ear in both genotypes, however only in WT mice did NIHL result in a significant increase in the proportion of units responsive to the spared ear in ipsilateral cortex (Naïve vs NIHL, WT-Left: p<0.001, KO-Left: p=0.0011, WT-Right: p=0.012, KO-Right: p=0.999, Tukey's HSD, FIG. 3A). Evoked firing rates were lower in KO animals (WT-Left-naive vs KO-Left-naive, p=0.0190, Tukey's HSD, FIG. 3B). Evoked firing rate and RF size showed similar patterns of changes following NIHL, i.e., decreases in both genotypes for contralateral stimulation, and increases only in WT animals for ipsilateral stimulation (Naive vs NIHL, mean evoked firing rate: WT-Left: p 0:001, KO-Left: p=0:166, WT-Right: p=0:045, KO-Right: p=1:00, FIG. 3B; RF size: WT-Left: p 0:001, KO-Left: p=0:0170, WT-Right: p=0:007, KO-Right: p=0:999, Tukey's HSD, FIG. 3C).

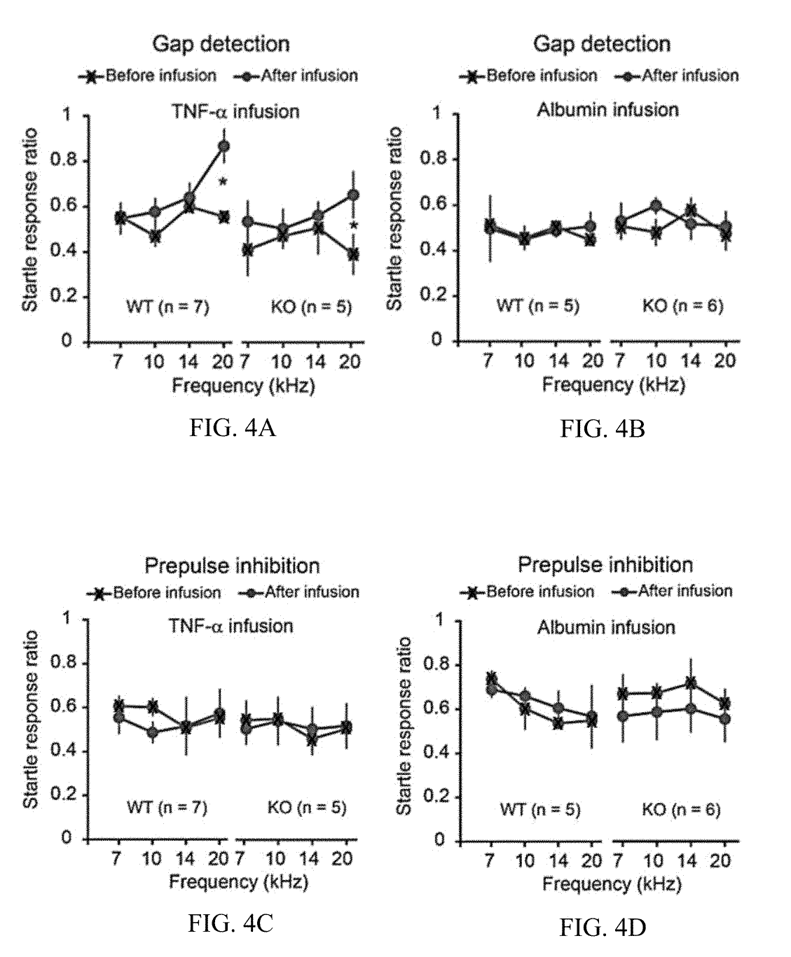

FIGS. 4A-4B demonstrate that TNF-α is sufficient to induced tinnitus. FIG. 4A shows that auditory cortical infusion of mouse recombinant TNF-α results in behavioral signs of tinnitus both WT and TNF-α KO mice, as indicated by impaired gap detection performance. FIG. 4B shows that infusion of mouse albumin as a control did not result in tinnitus. FIGS. 4C-4D show that prepulse inhibition was not altered by infusion of TNF-α or albumin.

### Example 3: Cortical Infusion of Recombinant TNF-α Results in Tinnitus

To test whether TNF-α is sufficient to cause tinnitus symptoms, mouse recombinant TNF-α was infused into the right hemisphere auditory cortex of normal-hearing WT and TNF-α KO mice. Control WT and KO mice were infused with carrier solution containing artificial cerebrospinal fluid and mouse albumin. Gap detection and PPI performance was examined in three daily sessions prior to the injection and only the third session was used as the baseline performance. Mice were tested again after 3 days of post-surgical recovery. Gap detection performance was analyzed with a 4-way ANOVA on genotype (WT vs. KO), treatment (before vs. after infusion), drug (TNF-α vs. albumin) and frequency of the background tone. There were main effects of treatment (F_{1,152}=8.619, p=0.0038) and drug (F_{1,152}=4.476, p=0.032). There was also treatment×drug interaction (F_{1,152}=5.730, p=0.018), indicating that TNF-α and albumin changed gap detection performance differently. However, the interaction was independent of genotype (treatment×drug×genotype interaction, F_{1,152}=0.007, p=0.94) suggesting that TNF-α infusion had similar effects on both WT and KO mice. Posthoc t-test indicates that TNF-α significantly impaired gap detection at 20 kHz (WT: t₁₂=4.19, p=0.0013; KO: t₁₂=2.45, p=0.035), but not at other frequencies.

A similar 4-way ANOVA on PPI failed to show significant treatment×drug interaction (F_{1,152}=0.391, p=0.53) indicating that TNF-α did not alter PPI performance (FIG. 4).

### Example 4: TNF-α KO Mice do not Show Salicylate-Induced Tinnitus

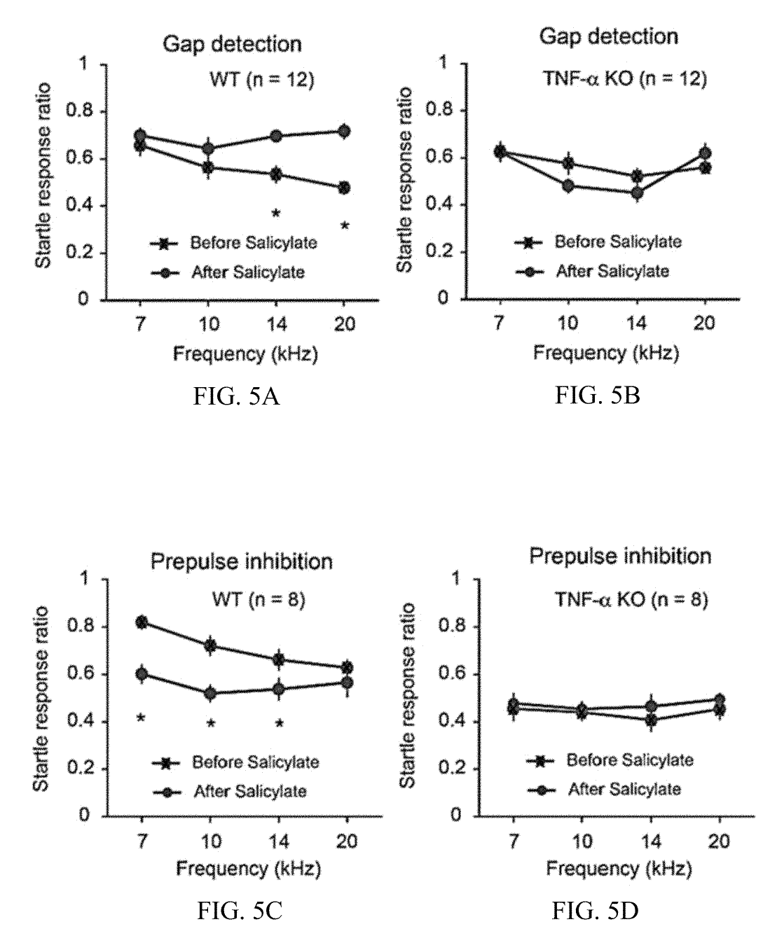

Salicylate has been shown to increase TNF-α expression in the inferior colliculus. Experiments were performed to examine whether TNF-α is required for salicylate-induced tinnitus using TNF-α KO mice. Systemic injection of 300 mg/kg salicylate resulted in robust behavioral manifestation of tinnitus 30 min later in wildtype mice (FIG. 5A; treatment×frequency 2-way ANOVA, treatment effect, F_{1,88}=24.28, p<0.0001; interaction, F_{3,88}=2.749, p=0.048). However, TNF-α KO mice did not show tinnitus after administration of the same dose of salicylate (FIG. 5B; treatment×frequency 2-way ANOVA, treatment effect, F_{1,88}=0.96, p=0.33; interaction, F_{3,88}=1.685, p=0.18). FIGS. 5C-5D show that systemic salicylate injection affected PPI performance in WT but not KO mice.

### Example 5: Blast Exposure Results in Traumatic Brain Injury Induced Tinnitus

A rat model of blast-induced tinnitus was developed to investigate the role of TNF-α in a traumatic brain injury (TBI) blast exposure induced tinnitus. Rats were anesthetized with isoflurane (0.75-1% in a 2:1 N₂O:O₂ gas mixture), placed on supportive netting, and secured with a locking device. All animals had one ear occluded with an earplug before they were blast-exposed in a shock-tube assembly (ORA, Inc.).

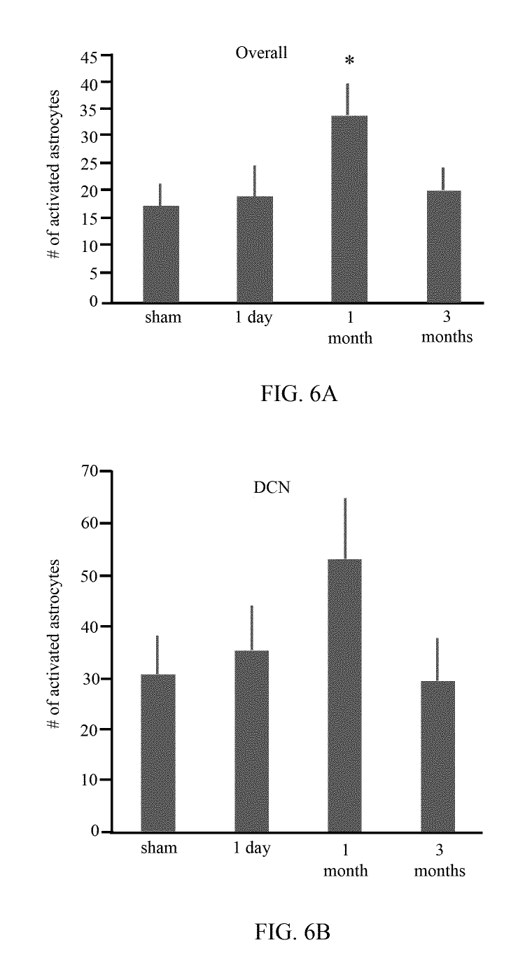

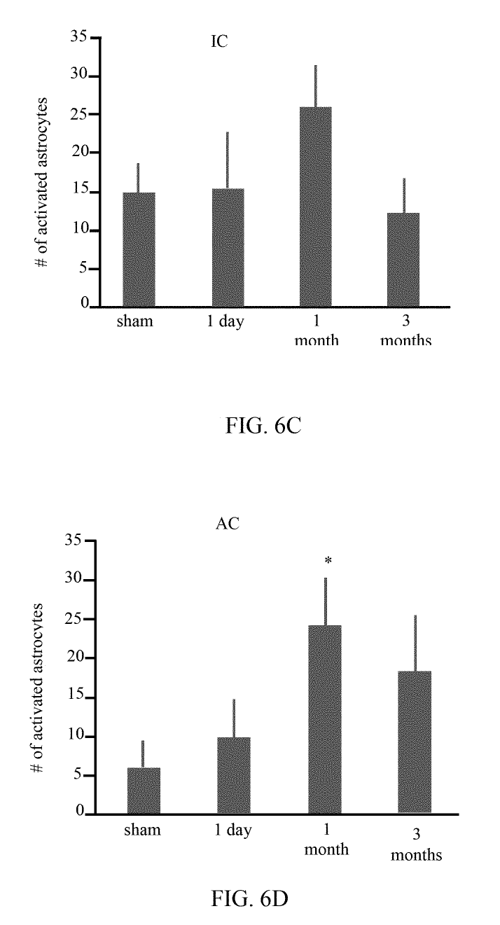

Magnetic resonance diffusion tensor imaging of the animals subjected to the blast exposure revealed evidence of brain trauma in the auditory pathway. Results revealed significant astrocyte activation in the auditory cortex (AC) and axonal degeneration and deformation in all major auditory brain regions (FIGS. 6A-6D). These results validate the blast-induced TBI model.

FIGS. 6A-6D demonstrate that blast exposure results in traumatic brain injuries. FIGS. 6A-D show the number of astrocytes in sham control and blast-exposed rats in the dorsal cochlear nucleus (DCN; FIG. 6B), inferior colliculus (IC; FIG. 6C), auditory cortex (AC; FIG. 6D) and all three regions combined (FIG. 6A). At 1 month after blast exposure, rats displayed a significant increase in the number of activated astrocytes in the AC and for all three regions combined (*, p<0.05). Intense glial fibrillary acidic protein (GFAP) staining was observed 1 month after blast exposure in the AC of blast-exposed rat as compared to sham control indicating increased GFAP expression. Blast-exposure resulted in swollen axons and axons with vacuoles and retraction balls as observed by axonal staining with silver impregnation.

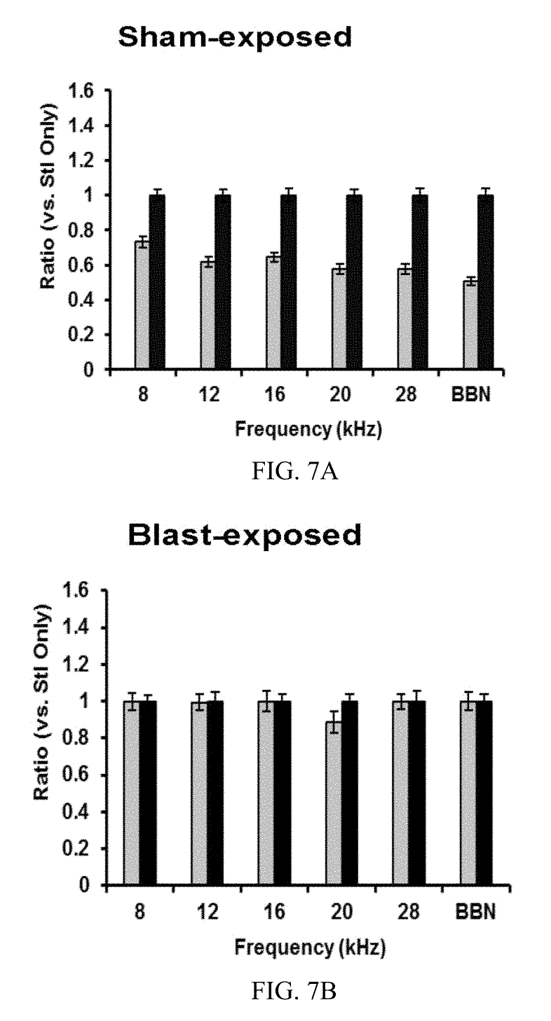

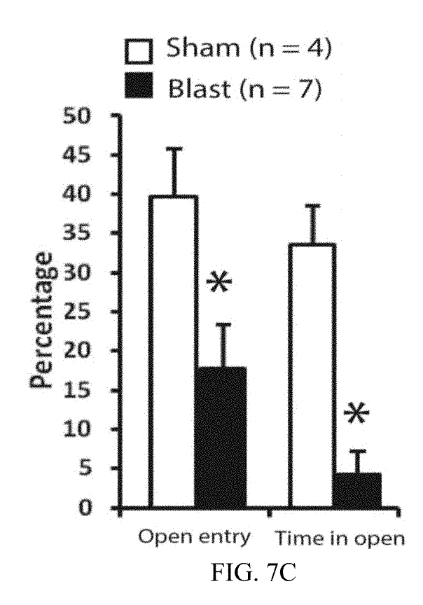

Behavioral evidence of blast-induced tinnitus was tested using a gap detection test as described above. Three 22-psi blasts given at 15-min intervals induced tinnitus, as revealed by impaired gap detection (FIG. 7B). Blast-exposed rats also spent less time in, and made fewer entries into, the open arms of an elevated plus maze (FIG. 7C), showing a higher anxiety level. The results are consistent with findings in blast-exposed humans and indicate that the blast model is suitable for studying mechanisms and treatment of blast-induced tinnitus and anxiety.

FIGS. 7A-C demonstrate blast-induced tinnitus and anxiety in the rat model of blast-induced tinnitus described above. FIG. 7A shows gap detection results indicating gap-induced suppression of the startle response (grey bars) in a control tinnitus(−) rat at 8, 12, 16, 20, and 28 kHz. The black bars represent startle only responses. FIG. 7B shows that gap detection is absent (grey bars) in a blast-exposed, tinnitus(+) rats, suggesting the presence of tinnitus at 8, 12, 16, 20 and 28 kHz (BBN, broad band noise). FIG. 7C shows that blast-exposed rats made fewer entries into, and spent less time in, the open arms of an elevated plus maze than did sham-exposed rats, indicating a higher level of anxiety. Asterisks (*) indicate statistical significance at p<0.05.

### Example 6: TNF-α Inhibitor Reduces Blast-Induced Tinnitus

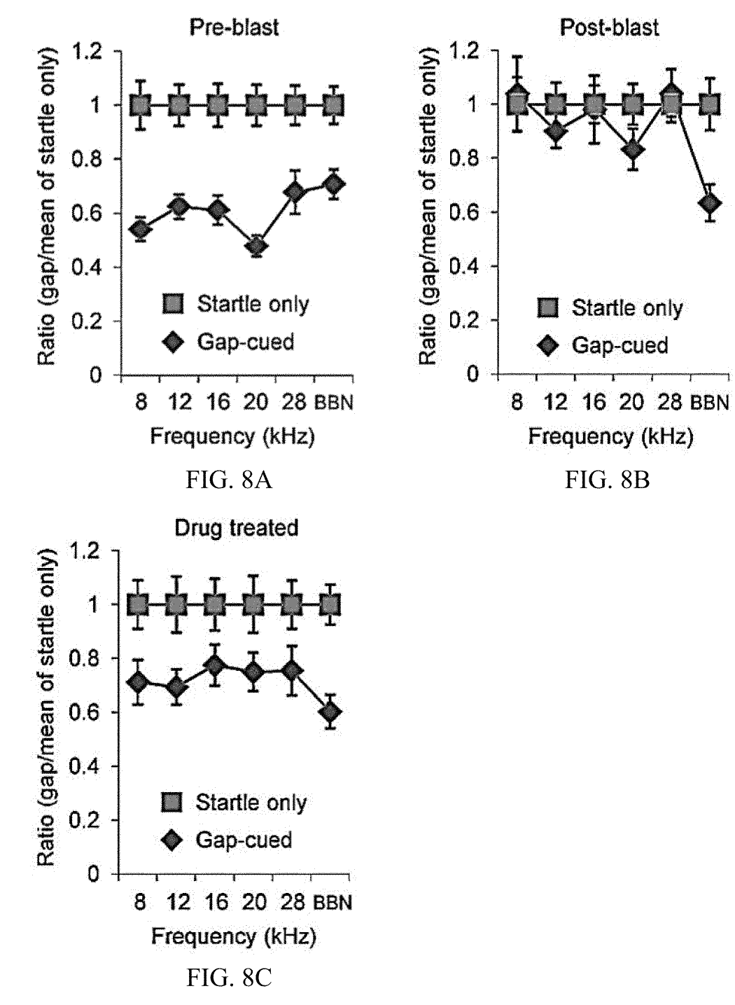

To demonstrate the involvement of TNF-α in blast-induced tinnitus, thalidomide, a TNF-α inhibitor, was injected into blast-exposed rats daily, for 5 days. Significant suppression of tinnitus was observed as revealed by improved gap detection (FIG. 8C). Untreated rats did not exhibit such improvements (FIG. 8B).

FIGS. 8A-C demonstrate the robust therapeutic effects of blocking TNF-α on blast-induced tinnitus. FIG. 8A shows that no tinnitus was present prior to blast exposure, as revealed by robust gap detection compared to startle-only response (Pre-blast, p<0.05, n=5). FIG. 8B shows that robust behavioral evidence of tinnitus was induced at many frequencies except for BBN, as revealed by significantly compromised gap detection after blast exposure (p>0.05, n=5). FIG. 8C shows that blast-induced tinnitus was significantly suppressed by administering thalidomide, a TNF-α inhibitor, for 5 days (drug-treated, p<0.05).

### Example 7: Administration of Thalidomide Reduces Noise Induced Tinnitus

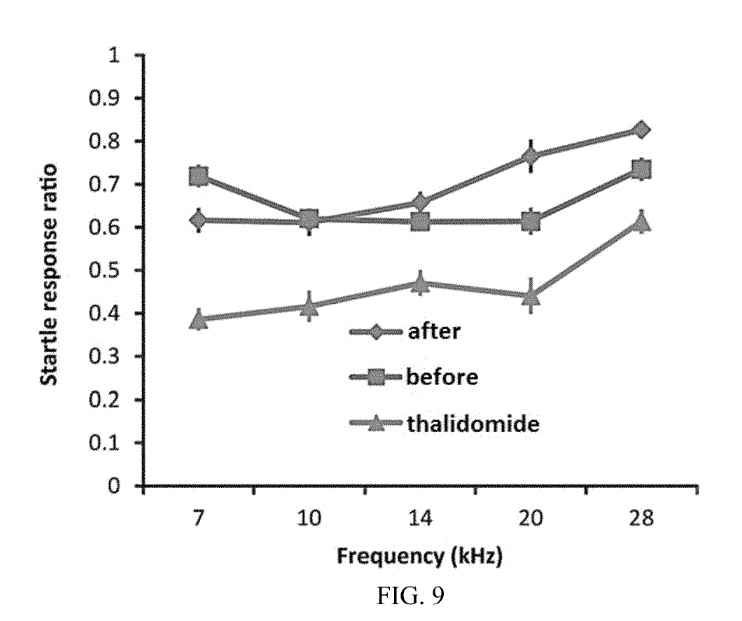

Eight C57BL/6 mice were tested with the gap detection task, described above, before and after undergoing NIHL. After noise exposure, gap detection performance was significantly impaired at 20 and 28 kHz, indicative of tinnitus at those frequencies (FIG. 9). Afterward, animals received daily injection of 100 mg/kg (IP) thalidomide for three days and were tested for tinnitus. The results indicate that thalidomide dramatically improved gap detection performance over a wide frequency range, completely reversing the impairment attributed to hearing loss-induced tinnitus (FIG. 9) consistent with abrogation of TNF-α in noise-induced tinnitus.

### Example 8: Administration of TNF-α Inhibitors for the Amelioration of Blast-Induced Tinnitus Secondary to TBI

Three TNF-α inhibitors, (etanercept, 3,6′-dithiothalidomide, and SSR150106) are administered and the pharmacological efficacy on inhibiting blast-induced TNF-α expression and therapeutic efficacies on blast-induced acute and chronic (˜3 month) tinnitus are evaluated. How etanercept, 3,6′-dithiothalidomide and SSR150106 affect blast-induced TNF-α expression is also evaluated.

To test pharmacological efficacies, rats are exposed to three blasts to induce tinnitus and TBI. Then, cochlear and brain tissues implicated in tinnitus etiology—including the DCN, IC, and AC—are sampled at different time points to determine the TNF-α mRNA and protein levels using Western blot and quantitative RT-PCR methods. Separate groups of rats will be given one of the three TNF-α inhibitors after blast exposure to determine their effects on reversing blast-induced changes in TNF-α expression.

To test the effects of blast exposure and TNF-α inhibitors on TNF-α expression in peripheral and central auditory pathways one hundred and eighty Sprague-Dawley rats are randomly assigned to 6 groups. Five groups undergo blast exposure with one ear protected, and the 6^{th} group undergoes a sham blast exposure procedure but is not exposed to blasts. Group 1 receives daily intraperitoneal (IP) administration of etanercept. Group 2 receives daily IP injection of 3,6′-dithiothalidomide. Group 3 receives daily IP injections of SSR150106. Drug doses are listed below. Group 4 is injected with saline as a control. Group 5, as a tinnitus-positive control, does not receive treatment. The sixth, non-blasted group serves as tinnitus-negative controls. Tissue samples are collected from the cochlea, DCN, IC, and AC of ten rats per group at 10, 30, and 90 days after the blast exposure. TNF-α inhibitors and control treatments are given for 5 days before tissue samples are collected (i.e., starting 5, 25, and 85 days after blast exposure). Real-time quantitative RT-PCR and Western blot are used to determine (a) the mRNA and protein levels of TNF-α increase in blast-exposed rats and (b) the pharmacological efficacies of the TNF-α inhibitors. Ten rats are used for each treatment and each time point.

TNF-α Inhibitors.

Etanercept is obtained from Wyeth Laboratories, Philadelphia, Pa. SSR150106 is provided by Sanofi. 3,6′-Dithiothalidomide is synthesized by CheminPharma (www(dot)cheminpharma(dot)com) according to Baratz et al. (2011) J Neurochem. 118(6):1032-42 and Luo et al. (2008) Synthesis. 21:3415-3422, the disclosures of which are incorporated herein by reference, to greater than 99.8% chemical purity. Effective doses of the drugs for rodent models of TBI (etanercept at 5 mg/kg; 3,6-Dithiothalidomide at 28 mg/kg; SSR150106 at 90 μg/kg) are initially used and subsequently modified according to subject response.

Blast Exposure.

The blast exposure procedure is essentially that of Mao et al. (2012) J Neurotrauma. 29(2):430-44, the disclosure of which is incorporated herein by reference. To induce chronic tinnitus and realistically reflect multiple blasts in war theaters, each rat undergoes three blast exposures at 15-min intervals using a shock-tube assembly (ORA, Inc.). Blast exposure at this level does not affect rats' eating and drinking behaviors. Rats are anesthetized with isoflurane (0.75-1% in a 2:1 N₂O:O₂ gas mixture). The right ear is occluded with an earplug and sealed with mineral oil. The shock tube, housed at the Wayne State University Bioengineering Center, is custom-built with a maximum working pressure of 100 psi (˜700 kPa). The device consists of a pressure chamber connected to a 20′ long, 12″ diameter hollow tube. Releasing the pressure in the chamber generates a single shock wave. The terminal end of the tube can be configured as open or capped. Keeping the tube open ended simulates a free-field shock wave; capping the end of the tube generates a more complex waveform. In this research, a single-blast, open-tube configuration is used. Parametric changes in shock-wave duration can be made by changing the length of the high-pressure chamber; the peak pressure can be varied by changing the thickness of the mylar membrane that separates the pressure chamber from the tube.

The experimental setup is configured with instruments to monitor the pressure waveform using piezoelectric sensors, with one sensor placed axial to the blast pressure source (137A22 Free-Field ICP Blast Pressure Sensor, PCB Piezotronics) and the other positioned perpendicular and threaded into the tubing to capture details of the induced pressure wave (1022A06 ICP Dynamic Pressure Sensor, PCB Piezotronics). An analog-to-digital data acquisition system (DASH 8HF, Astro-Med, Inc.) is in place to acquire/monitor data. A high-speed video camera (HG100K, Kodak), which captures up to 3000 frames/s, is placed at the open end of the tube to record the effects of the shock wave on the animal's orientation and movement, prior to, during, and after delivery of the pressure wave.

Behavioral Test for Tinnitus.

This procedure used as a behavioral assay for tinnitus is essentially that reported by Turner et al. (2006) Behav Neurosci. 120(1):188-95, Mao et al. (2011) J Neurotrauma. 29(2):430-44, Lobarinas et al, (2013) Hear Res., and Pace et al. (2013) PLoS One. 8(9):e75011, the disclosures of which are incorporated herein by reference. Each rat is acclimated to the custom-made restrainer for 2 hours/day for 4 days, so that they remain comfortable in the restrainer during subsequent gap detection and PPI tests. The gap detection test is carried out using a commercial system (Hamilton-Kinder Behavioral Testing Systems, Poway, Calif.). In a testing chamber, a piezoelectric transducer is attached underneath a restrainer platform to measure the startle force. Test stimuli are calibrated before each test session using a Newton Impulse Calibrator and a sound pressure level meter. For the gap detection procedure, each rat is tested in the chamber with a continuous background sound. The sound is 2,000-Hz wide bandpass noise centered on one of five frequencies (8, 12, 16, 28, 32 kHz) or broadband noises (8-32 kHz range). The background sound is presented at 60 dB sound pressure level (SPL). The startling noise burst (115 dB SPL, 50-ms duration) is presented through a second speaker. For rats with normal hearing, a silent gap in the background sound signals the forthcoming startle stimulus and inhibits the subsequent startle response. Rats with tinnitus have difficulty detecting the gap when the background sound is qualitatively similar to their tinnitus, resulting in less inhibition of their startle response compared with normal-tinnitus rats. 16 trials are performed for each background sound in each test session—8 gap-cued and 8 no-gap trials. The ratio of responses between the gap and no-gap conditions indicates how well the animal can hear the gap. A ratio close to 1 indicates that the rat cannot detect the gap. A smaller ratio indicates better gap detection.

The PPI test procedure is similar to that of the gap detection test, except that no background noise is given. Instead, a 60-dB, 50-ms prepulse sound is presented from 90 ms before the onset of the startle stimulus. The prepulse sound serves to signal the forthcoming startle stimulus and inhibit the startle response. The ratio of the startle responses in prepulse vs. no-prepulse trials is indicative of how well the animal detects the sound. If an animal loses hearing or is otherwise hearing-impaired at certain frequencies, the startle ratio is higher for those frequencies. The sounds used for the PPI test is identical to those in gap detection (8, 12, 16, 28, and 32 kHz, and broad-band noise, shaped with 2-ms rise/fall ramps). Each test lasts approximately 30 minutes.

Western Blot.

Rats are euthanized with an overdose of isoflurane. Tissues are collected from the AC, IC, and DCN based on cranial and brain landmarks. The isolated tissues are homogenized in lysis buffer containing 50 mM Tris-HCl (pH 7.4), 150 mM NaCl, 1% Nonidet P-40, and protease inhibitor cocktail (Roche). After gentle rotation for 3 h at 4° C., homogenates are centrifuged at 14,000×g for 60 min at 4° C. and the supernatants collected. Protein lysate (100 μg) is dissolved in SDS sample buffer containing 5% β-mercaptoethanol and heated to 95° C. for 5 minutes. Equal amounts of proteins are loaded on 10% SDS-PAGE gels and transferred to nitrocellulose membranes. Membranes are blocked in 1× Western Blocking Reagent (Roche) in 0.1% PBS-Tween 20 (PTN) for 1 h at 4° C. and incubated with a 1:500 dilution of the anti-TNF-α or anti-α-tubulin primary antibody in PTN overnight at 4° C. Afterwards, the blots are washed with 0.1% PTN and incubated with horseradish peroxidase-conjugated goat anti-rabbit or anti-rat antibodies (1:5,000) (Jackson ImmunoResearch) in PTN for 2 h at 4° C. Immunoreactive bands are observed using an enhanced chemiluminescence system (NEN). TNF-α band intensities are normalized to the band intensities of α-tubulin, which is used as a loading control. Membrane-bound pro-TNF-α and soluble TNF-α are differentiated by their size and quantified separately.

qRT-PCR.

Total RNA samples are prepared from the tissues with RNA Wiz (Ambion). The total RNA obtained (3 μg) is reverse-transcribed using a first-strand cDNA synthesis kit (BD Biosciences, Palo Alto, Calif.). qRT-PCR is performed using an ABI CYBR Green PCR Kit (Qiagen). TNF-α-specific fragments are amplified. PCR reactions are done under the following cycling conditions: an initial denaturation at 94° C. for 3 min followed by 25-40 cycles at 95° C. for 45 sec, annealing at 53° C. for 45 sec, and elongation at 72° C. for 1 min with a final elongation step at 72° C. for 10 min. The 18S rRNA gene is used as an internal standard (QuantumRNA, Ambion).

Immunohistochemical Staining for Glial Fibrillary Acidic Protein (GFAP) to Analyze Gliosis.

This procedure is modified from that presented by Yang et al. (2011) Proc Natl Acad Sci USA. 108(36):14974-9 and Shibuki et al. (1996) Neuron. 16(3):587-99, the disclosures of which are incorporated herein by reference. After sham or blast exposure and drug or saline treatment, each rat is euthanized by lethal dose of isoflurane (5% v/v) and perfused transcardially with 4% paraformaldehyde in 0.1M PBS (pH 7.4). The brain is removed, post-fixed, and subsequently cryoprotected (30% sucrose in 0.1M PBS, pH 7.4). Next, 50-μm thick frozen (−22° C.) serial coronal sections encompassing the DCN, IC or AC are cut and collected in 1× phosphate buffered saline (PBS). DCN sections are collected between −10.68 and −11.76 from the bregma, IC −7.80 and −9.24, and AC −4.08 and −6.84.

For quantitative analysis of gliosis in DCN, IC and AC regions, 5 representative sections per animal from each region are incubated in citrate buffer (pH 6.0) at 90° C. for 1 hour followed by immersion in 0.3% hydrogen peroxide to quench endogenous peroxidase activity. The sections are then incubated overnight in a mouse anti-GFAP (Glial fibrillary acidic protein) antibody (NE1015, EMD Chemicals, Gibbstown, N.J.) diluted in 2% normal goat serum (Vector Laboratories, Burlingame, Calif.) in 1% bovine serum albumin. The sections are then be incubated in biotinylated anti-mouse IgG (Vector Laboratories, Burlingame, Calif.) followed by exposure to Vectastain Elite ABC reagent and chromogen development by diaminobenzidine. In control incubations, normal goat serum is substituted for the primary antibody.

GFAP-reactive astrocytes are counted microscopically. Ten digital images (×20 magnification) from each section encompassing bilateral regions of the DCN, IC, or AC are obtained. Then the total number of identifiable astrocytes in each digital image is counted by a blinded observer using the cell counter function in ImageJ (rsb(dot)info(dot)nih(dot)gov/ij/). The average number of astrocytes per group and region are calculated and statistically compared for group-wise differences using one-way analysis of variance or t-test with SPSS. Unbiased stereological techniques are employed in sample selection and cell counting.

Silver Staining for Degenerating Axons.

For qualitative analysis of the extent of blast-induced axonal injury in sections encompassing the DCN, IC, and AC, a separate set of 5 representative sections from each region per rat are subjected to a silver impregnation technique. The sections are immersed for 3 min in pretreatment solution (equal volumes of 9% sodium hydroxide and 15% hydroxylamine), washed in 0.5% acetic acid (3×3 min), incubated in an impregnation solution (5 mg/ml ferric nitrate and 100 mg/ml silver nitrate) for 30 min, washed in 1% citric acid (4×2 min), and washed in 0.5% acetic acid for 5 min. Then they are placed in developer solution until they turn pale gray. After sufficient development, they are removed and washed thoroughly in 0.5% acetic acid (3×10 min), rinsed in distilled water, mounted on a slide, cover-slipped and examined under a light microscope for degenerating axons.

Gliosis and axonal degeneration is compared between the two sides to distinguish shockwave effects (occurring in both sides) from hearing loss effects (affecting mainly exposed ear but not the protected ear).

Elevated Plus Maze Test for Anxiety.

The anxiety level of blast-exposed rats is measured with the elevated plus maze (Tracoustics Inc., Austin, Tex.). The light level in the open arms is set at 1.5 lux and in closed arms at 0.09 lux. A camcorder is mounted on the ceiling above the maze to record rats' behaviors on the apparatus. Prior to testing, rats are handled 2 minutes/day for 5 days by the same experimenter conducting the tests. Rats are transported to the testing room and acclimated to the dim light for 4 hours before testing. Each rat is placed on an open arm facing away from the center of the maze. The rat's behavior is recorded for 5 min. After the test, the rat is returned to its cage and the maze cleaned with 70% alcohol followed by 5 min of air drying. Each rat is tested for one trial only. Two “blind” and experienced experimenters score the videos independently and the average score is taken for each measurement. Anxiety level is quantified as the percentage of time the rat spends on the open arms within the 5-min time frame and the percentage of entries the rat makes into the open arms.

Patch Clamp Electrophysiological Recording.

Standard patch-clamp recording methods are used.

Multi-Channel Chronic Recording.

Chronic electrode arrays are implanted in the left DCN, right IC and right AC under isoflurane anesthesia. All arrays are dipped in 3% DiI solution to label the tracks of implantation. To implant in the DCN, a craniotomy is performed and a 16 (2×8) microwire array is lowered 100-150 μm below the DCN surface. To implant in the IC, a two-shank 32-Channel NeuroNexus probe (C2×16-5 mm 100-403) is inserted in a coronal plane along a dorsolateral to ventromedial trajectory at a 30-45° angle relative to the parasagittal plane. To implant in the primary auditory cortex (AI), a 16 (2×8) microwire array is implanted 2.7 to 5.8 mm posterior to the bregma, and ˜0.8-1.0 mm from the cortical surface. The AI is defined by its short response latency (8-20 ms) and its continuous tonotopy. Finally, arrays are secured to the skull with dental acrylic. The rat's body temperature is maintained at 37° C. Immediately after surgery, electrophysiological recording from the AC, IC and DCN will be conducted to document the physiological status. Further experiments are conducted following a ˜10-day recovery period from surgery. Spontaneous and sound-evoked activity is recorded in awake rats before, during and after blast exposure. Neural signals are preamplified and bandpass-filtered (300-3,000 Hz), and thresholded (1.5 times the root mean square level). Finally, the neural output is fed into a 128-channel auditory workstation (RZ-2, OpenEx software, TDT). Spontaneous activity and frequency tuning curves (FTCs) are monitored daily from before to up to 3 months after blast impact. FTCs are used to demonstrate frequency representations of each recording electrode/channel. Stimulus-driven activity in response to noise and tone bursts (100 ms duration, 1 burst/sec, 0-80 dB SPL) is recorded to establish post-stimulus time histograms for response patterns. (i) Single- and multi-unit spontaneous activity. Spontaneous activity is recorded from and compared between blast- and sham-exposed rats, and between drug- and saline-treated rats. (ii) Stimulus-driven activity and FTCs. FTCs are acquired to determine tonotopic frequency representations and plastic reorganization. Tones (50 ms in duration, 2-44 kHz, 0-85 dB SPL, incremental steps of 5 dB) are delivered to the left ear by a Fostex T90a super tweeter. PSTHs are generated for temporal classification using characteristic frequency (CF) tone and BBN bursts. Tone and noise bursts (50 ms duration, 1/s, 2.5 ms rise/fall times) at 10 dB above threshold are repeated 100 times at 0-85 dB SPL in 7-dB increments.

Statistical Tests and Power Analysis.

ANOVA is performed to evaluate statistical significance. The sample sizes in the proposed experiments were chosen to ensure sufficient statistical power for robust results. Initial results indicate that the ratio of the gap-cued startle response over the uncued startle response has a standard deviation of σ≈0.13. The mean size of the effect of blast-induced tinnitus on the ratio is δ≈0.20. Comparison is typically made between two groups (k=2, saline vs. drug) using ANOVA. With each group having 10 animals and the size of type I error set at <0.05, the noncentrality is Φ(1, 28)=(δ/σ) sqr(n/(2k))=2.4, corresponding to a statistical power >0.8.

To test therapeutic efficacies of different TNF-α inhibitors on blast-induced tinnitus, TBI and anxiety, after blast exposure, rats are tested behaviorally for evidence of tinnitus, and the three TNF-α inhibitors are tested for therapeutic effects on the induced tinnitus. The TNF-α inhibitors are also further tested for blast-induced TBI and anxiety.

The effects of etanercept, 3,6′-dithiothalidomide, and SSR150106 on blast-induced acute and chronic tinnitus are tested in a total of 180 rats (i.e., 60 rats/drug). The following describes the experiments testing the effects of a single drug on blast-induced tinnitus. Sixty rats are randomly assigned to 6 groups—3 groups receive drug treatment, and 3 corresponding control groups receive saline/vehicle injections. The experimenters are “blind” to the treatment status of the rats. All rats undergo a gap detection test to obtain stable baseline data. Afterward, the 1^{st} drug-treated group (tinnitus prevention) receives up to 5 consecutive days of drug administration (single injection daily). During that time, the gap detection task is performed daily to determine the effects of the drug on gap detection behavior. After the last gap detection test, rats undergo blast exposure. The drug administration continues for 5 more days after blast exposure. This group of rats is tested for tinnitus 10 and 20 days after the end of drug administration to determine whether the drug prevented the induction or expression of tinnitus. If and when the drug alters gap detection behavior in rats with normal hearing, a group of TNF-α KO mice is introduced as a control for potential off-target effects. The 2^{nd} drug-treated group (acute tinnitus) of rats receive blast exposure first and then start receiving the drug 10 days after the exposure; the drug is administered once daily for 5 days. Starting from the last day of drug administration, rats are tested for tinnitus every other day for 10 days. The 3^{rd} drug-treated group (chronic tinnitus) undergo blast exposure, and then at 90 after the exposure, the rats are given daily injections of the drug for 5 days. Starting from the last day of drug administration, rats are tested for tinnitus for a period of 20 days to determine to what extent the drug abolishes chronic tinnitus that had been induced 3 months prior to drug treatment and the duration of the drug effects. Drug is given up to 1 month if tinnitus persists after 5 days of treatment.

The effects of TNF-α inhibitors on blast-induced TBI are evaluated in thirty rats. The rats are randomly assigned to a drug, a saline, or a sham-blasted group. The drug-treated and saline-treated groups undergo blast exposure. Rats receive daily drug or saline injection essentially for five days starting essentially on the day of blast exposure. The sham-blasted group also receives saline injections. All rats are then euthanized and their brains processed by immunohistochemistry and silver staining (see above) for examination of gliosis and axonal damage.

The effects of TNF-α inhibitors on blast-induced anxiety are evaluated in thirty rats randomly assigned into three groups sham—blasted/no-injection, drug, and saline. Baseline anxiety levels are assessed with the elevated plus maze test. Then, the drug- and saline-treated groups undergo blast exposure and anxiety levels are measured to quantify blast-induced anxiety behavior. Rats receive daily drug or saline injections for essentially five days starting essentially on the day of blast exposure. Afterward, all rats are evaluated for anxiety level with an elevated plus maze test.

To characterize how TNF-α inhibitors regulate synaptic transmission and neuronal activity in the auditory pathway of rats with blast-induced tinnitus and how pharmacological modulation of TNF-α activity affects blast-induced changes in synaptic transmission and neuronal activity, both in vitro and in vivo synaptic transmission and neuronal activity levels are determined. Blast-exposed rats receive the selected TNF-α inhibitor. Synaptic transmission in vitro with patch-clamp recording from auditory cortical slices is examined. Neural activity is also recorded in vivo from the DCN, IC, and AC in awake and behaving rats to investigate spontaneous activity, burst firing, neuronal synchrony, and sensory map reorganization. The results elucidate the mechanisms underlying TNF-α-mediated treatment for blast-induced tinnitus.

The effects of blast exposure and TNF-α inhibitors on excitatory and inhibitory synapses and membrane excitability of auditory cortical neurons, in vitro, is evaluated in brain slices from eighty Sprague-Dawley rats randomly assigned to 8 groups. Three groups receive identical drug treatment and three groups receive saline daily starting at 5, 25, or 85 days after the blasts, for a period of 5 days. A separate drug group and saline group do not undergo blast exposure but are given TNF-α inhibitor or saline for 5 days, and serve as naïve/tinnitus-negative controls. One day after the final drug administration, rats are euthanized, brain slices are prepared from bilateral auditory cortices, and synaptic and membrane properties of cortical pyramidal neurons are examined by patch-clamp electrophysiology as described.

The effects of blast exposure and TNF-α inhibitors on spontaneous and evoked activity of neurons in the auditory pathway, in vivo, is evaluated in thirty rats randomly assigned to 3 groups. Two groups undergo blast exposure, and the 3^{rd} group undergoes sham exposure. All rats are implanted with three sets of recording electrodes in three brain regions DCN, IC and AC. Spontaneous and sound-evoked neural activity is recorded simultaneously from DCN, IC and AC over a period of 3 months. Starting at essentially 30 days after the blast exposure, rats in one of the blast-exposed groups receive daily administration of the selected TNF-α inhibitor for essentially 5 days. Rats in the remaining blast-exposed group and the sham-exposed group receive daily saline injections. Neural activity levels are recorded and compared with the pre-drug treatment level, between the drug- and the saline-treated groups, and between the blast- and sham-exposed groups. Drug administration is, in some instances, extended up to 1 month depending on its effects on tinnitus and the putative neural correlates of tinnitus and subject response.

### Example 9: TNF-α Silencing for the Amelioration of Tinnitus

One hundred and twenty mice are randomly assigned into eight groups. Tinnitus is measured with the gap detection test, and hearing and general auditory processing are examined with prepulse inhibition. All animals undergo gap detection and the PPI test to establish baseline behavior measures. Seven groups are then noise-exposed with one ear protected to induce tinnitus, and the remaining group serves as a naive/tinnitus negative control. Gap detection and PPI are tested again after noise exposure to confirm the presence of tinnitus. Afterward, one of the noise-exposed groups receives a microinjection into the right auditory cortex of a lentivirus carrying a shRNA against TNF-α to silence its expression. A second noise-exposed group receives a microinjection of a lentivirus that contains scrambled sequences as a control group. For comparison, a third exposed group receives an infusion of etanercept into the right cerebroventricle via an osmotic pump. A fourth exposed group receives an infusion of mouse albumin fraction V as control. For comparison, a fifth exposed group receives a daily IP injection of thalidomide (100 mg/kg). A sixth exposed group is injected with a vehicle solution as a control group. The seventh exposed group serves as noise-exposed/tinnitus positive control. Gap detection and PPI tests are performed to determine to what extent the treatments alleviate blast-induced tinnitus.

Brain tissues of the treated mice are analyzed by Western blot and RT-PCR to evaluate to what extent the treatments block brain TNF-α function.

Post-transcriptional gene silencing used to knockdown TNF-α expression is achieved using a lentivirus expressing an shRNA (Santa Cruz Biotechnology, Inc.). A lentivirus expressing a scrambled sequence is used as a control. After noise exposure, animals are anaesthetized with ketamine (100 mg/kg IP) and xylazine (10 mg/kg IP), and virus is injected into the right auditory cortex. A burr hole is made on the temporal ridge 1.75 mm anterior from the junction between the temporal ridge and the transverse suture to expose the primary auditory cortex. A micropipette filled with the virus solution is lowered down 500 μm from the pial surface, and 1 μl virus solution is injected at 100 nl/min by pressure injection (Stoelting Quintessential Injector, Wood Dale, Ill., USA). The micropipette is then retracted 250 μm and an additional 1 μl virus solution is injected. The micropipette is left in place for 8 min at the end of each injection to minimize leaking. After injection, the skin is sutured, and the animals are returned to their home cages after regaining movement.

Intracerebroventricular infusion of Etanercept is achieved by a mini osmotic pump (0.5 μl/h, 1 week) and infusion cannula (Alza), assembled as directed, and filled either with vehicle of synthetic CSF and mouse albumin (Fraction V, 1 mg/ml; MP Biomedical), or Etanercept dissolved in vehicle solution. The cannula is implanted in the right auditory cortex. Etanercept at a dose of 5 mg/kg administered through the I.P. route attenuates TBI. In some instances, direct infusion, e.g., into the brain, of Etanercept is preferred. The osmotic pump is implanted immediately after noise exposure and 0.5 mg/kg/24 h is infused into the right cerebroventricle for 5 days.

Behavioral tests for tinnitus and behavioral tests for hearing functions and startle reflex are performed as described herein.

### Example 10: 3,6′-Dithiothalidomide Partially and Significantly Suppresses Blast-Induced TNF-α Expression at the Protein Level

Animal Subjects

40 adult male Sprague Dawley rats (100-120 day old at the beginning of experimentation) were purchased from Charles River Laboratories (Kingston, Va.).

TNF-α Inhibitory Agent

The therapeutic drug used was 3,6′-dithiothalidomide, which was synthesized according to a published procedure to greater than 99.8% chemical purity (Baratz et al., 2011; W. Luo et al., 2008). The drug was freshly prepared prior to each study. 3,6′-dithiothalidomide was prepared as a suspension in 1% carboxymethyl cellulose to provide a final concentration of 28 or 56 mg/kg (0.1 mL/10 g and 0.1 mL/100 g body weight injection in mice and rats, respectively), and was administered intraperitoneally (i.p.). These concentrations of 3,6′-dithiothalidomide are equimolar to 25 and 50 mg/kg of thalidomide.

Blast Exposure

Each of the rats was subjected to blast exposure in the left ear using a shock tube (ORA Inc.) located at the Wayne State University Bioengineering Center. The blast exposure of each rat was performed under anesthesia with a mixture of air (1 liter/min) and isoflurane (5% v/v). The rat was placed on supportive netting with a metal surround and secured on a pole with its head facing the oncoming shockwave. During blast exposure, the rat's right ear was occluded with a silicone earplug (Mack's; McKeon Products, Warren, Mich.), followed by application of mineral oil to seal any remaining openings. The average energy under 10 kHz measured at 22 psi was equivalent to 150 kPa or 197.5 dB SPL. After blast exposure, each rat was transferred to a polycarbonate cage.

Administration of TNF-α Inhibitory Agent and Analysis

Two exposed groups received either 56 mg/kg 3,6′-dithiothalidomide i.p. or vehicle for two days starting on the same day of the blast exposure. Another two exposed groups received 56 mg/kg 3,6′-dithiothalidomide i.p. or vehicle treatment for five days starting on the day of the blast exposure. Two more exposed groups received 56 mg/kg 3,6′-dithiothalidomide i.p. or vehicle treatment for five days starting on the fifth day after blast exposure. On the last day of the drug/vehicle injection, brain samples were collected. ELISA and Western blot was performed to determine the effects of blast and 3,6′-dithiothalidomide treatment on TNF-α protein levels.

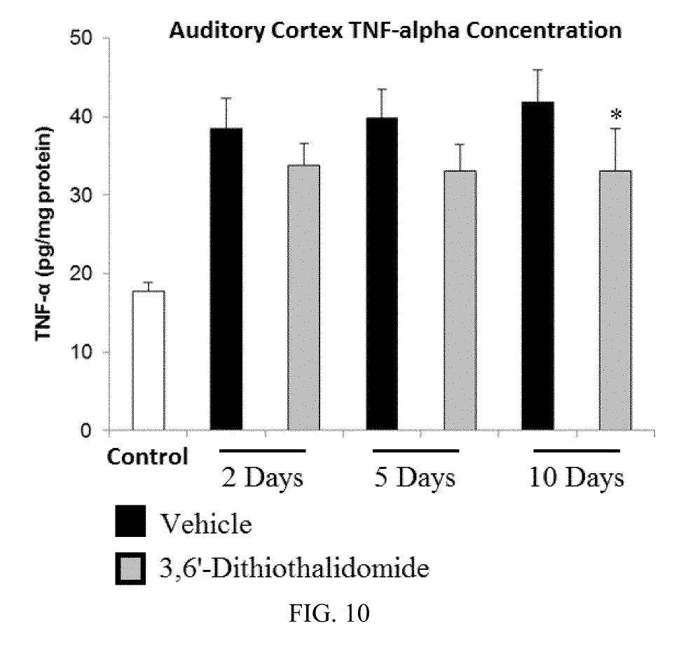

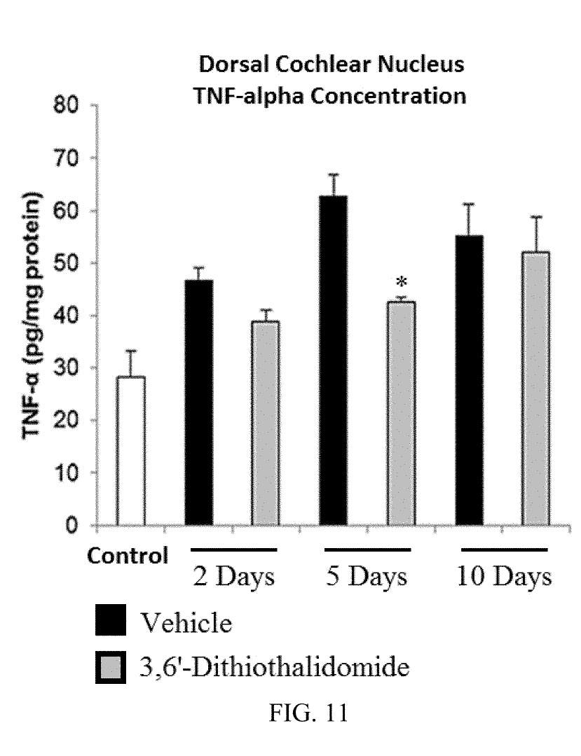

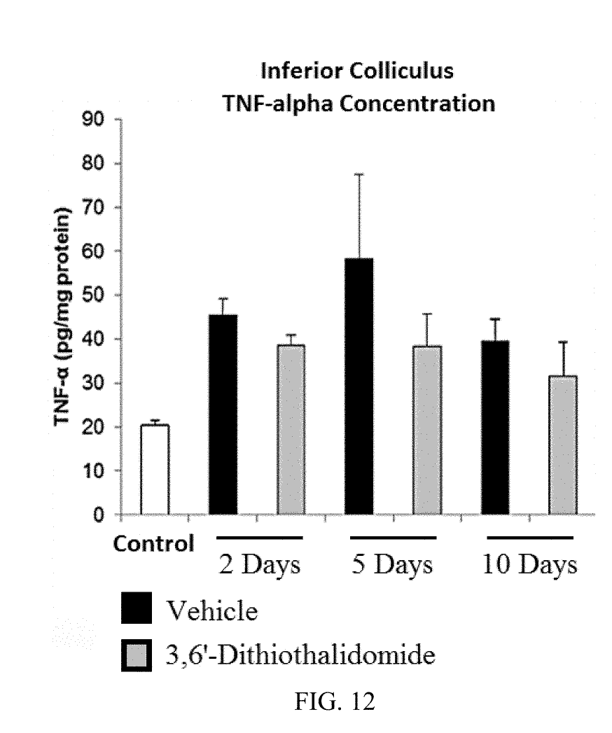

TNF-α protein levels increased significantly in the AI, DCN and IC 2, 5 and 10 days after blast exposure (FIGS. 10-13). Administration of 3,6′-dithiothalidomide partially reduced TNF-α protein levels (FIGS. 10-13). The reduction reached statistical significance in the AC and DCN on the fifth day after blast exposure (FIGS. 10 and 11; statistical significance at P<0.05 indicated by asterisks).

FIG. 10 demonstrates that 3,6′-dithiothalidomide treatment reduced TNF-α protein levels in blast-exposed rat AC as measured with ELISA. An overall significant difference between the drug and vehicle groups was observed. The reduction in TNF-α protein levels displayed statistical significance (P<0.05) in the individual 3,6′-dithiothalidomide group that was treated for five days starting on the fifth day after blast exposure (10 Days) relative to the corresponding vehicle-treated group.

FIG. 11 demonstrates that 3,6′-dithiothalidomide treatment reduced TNF-α protein levels in blast-exposed rat DCN as measured with ELISA. An overall significant difference between the drug and vehicle groups was observed. The reduction in TNF-α protein levels displayed statistical significance (P<0.05) in the individual 3,6′-dithiothalidomide group that was treated for five days starting on the day of blast exposure (5 Days) relative to the corresponding vehicle-treated group.

FIG. 12 demonstrates that 3,6′-dithiothalidomide treatment reduced TNF-α protein levels in blast-exposed rat IC as measured with ELISA. This effect, however, did not achieve statistical significance.

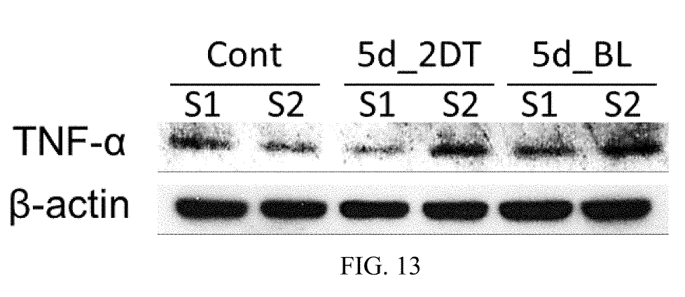

FIG. 13 displays a Western blot analysis of rat AC for control rats (Cont), rats receiving 3,6′-dithiothalidomide for five days starting on the day of blast exposure (5d_2 DT), and rats receiving vehicle for five days starting on the day of blast exposure (5d_BL).

The transcriptional/mRNA increase was transient only in the first two days, but the translational/post-translational increase in the protein level lasted much longer, suggesting that there was a translational/posttranslational increase in the TNF-α protein level. The present results indicate that 3,6′-dithiothalidomide cannot block such an increase. The effects of 3,6′-dithiothalidomide on the TNF-α protein level is likely secondary, for example, to its effects on blocking TNF-α mRNA increase. Never-the-less, a significant reduction in TNF-α protein levels in the AC and DCN was observed.

### Example 11: 3,6′-Dithiothalidomide Significantly Reduces Blast-Induced Tinnitus

Thirty two rats from Example 10 were used. Behavioral tinnitus testing, including gap detection and PPI testing, was conducted with Hamilton-Kinder startle-reflex hardware and software. During behavioral testing, each rat was placed in an individual noise-attenuation chamber to perform gap and PPI testing. In the gap procedure, each rat was presented with a constant 60-dB SPL background noise composed of 2,000 Hz bandpass signals at 6-8, 10-12, 14-16, 18-20, or 26-28 kHz or broadband noise (BBN; 2-30 kHz). A 115 dB SPL, 50 ms noise-burst was used as the startle stimulus to induce the acoustic startle reflex. The background noise session contained two conditions, one condition was the startle only condition in which rat was presented with the startle stimulus alone, and the other condition was the gap condition in which a rat was presented with a startle stimulus preceded by a silent gap embedded within the background noise. Silent gaps were 40 ms in duration with a lead interval of 90 ms to the startle stimulus. The startle reflex of rats was measured in response to 3 conditions: 1) background noise alone, 2) startle only, and 3) gap. Detection of the gap results in a reduction in the startle amplitude. Development of tinnitus in a specific frequency band impairs gap detection in the corresponding gap-carrier band, and thus, the startle amplitude reaches amplitudes elicited without the gap. For the PPI procedure, the parameters were almost the same as with gap except that no background noise or silent gap was used. The startle amplitude of rats in response to the two conditions was measured: the startle-only condition and a prepulse followed by the startle stimulus. In the latter condition, a 60-dB SPL, 40-msec prepulse was introduced 90 msec before the startle stimulus. The acoustic startle reflex of the rat decreased in response to the prepulse except when the rat had hearing loss at a frequency similar to the prepulse.

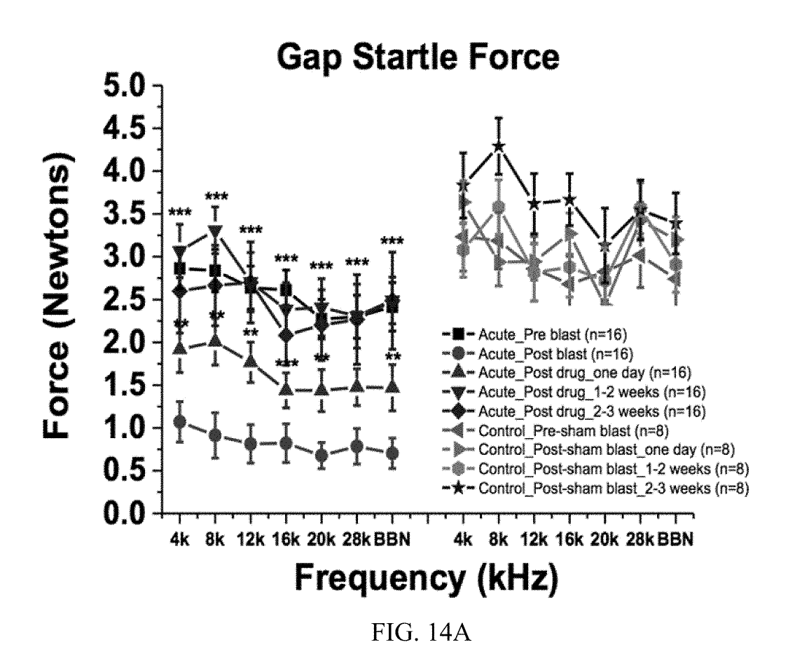

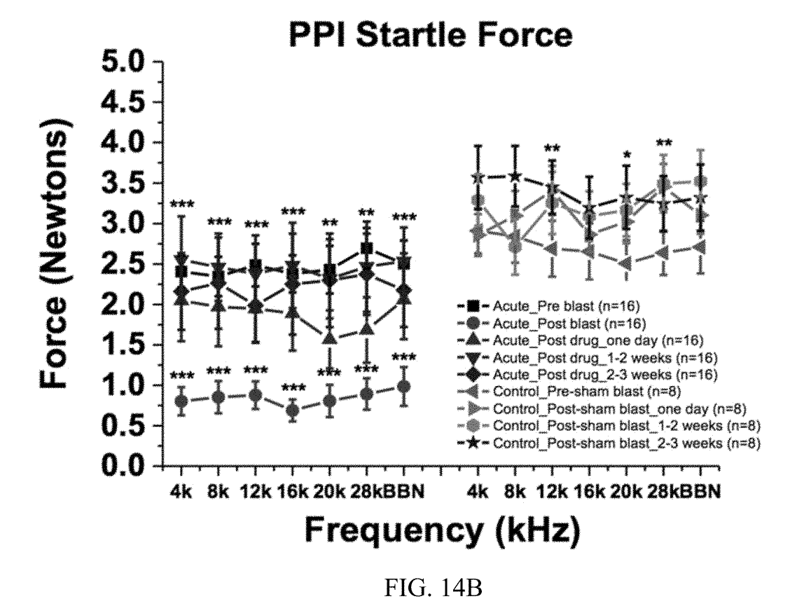

FIG. 14A shows that the startle force of the startle-only condition for the gap test exhibited a significant decrease at all frequencies in acute tinnitus rats (Acute_Post blast). After treatment with 3,6′-dithiothalidomide (56 mg/kg i.p.), the startle force recovered significantly compared to post blast (Acute_Post drug). Asterisks over the “Acute_Post blast” data points indicate differences relative to iso-frequency “Acute_Pre blast” data corresponding to p<0.001 for each pair of points (***). Asterisks over the “Acute_Post drug_one day” data points indicate differences relative to iso-frequency “Acute_Post blast” data with ** corresponding to p<0.01 and *** corresponding to p<0.001. No significant changes were observed in control rats (Control_Pre-sham blast and Control_Post-sham blast).

FIG. 14B shows that the startle force of the startle-only condition for the PPI test exhibited a significant decrease at all frequencies in acute tinnitus rats (Acute_Post blast). After treatment with 3,6′-dithiothalidomide (56 mg/kg i.p.), the startle force recovered significantly compared to post blast (Acute_Post drug). Asterisks over the “Acute_Post blast” data points indicate differences relative to iso-frequency “Acute_Pre blast” data corresponding to p<0.001 for each pair of points (***). Asterisks over the “Acute_Post drug_one day” data points indicate differences relative to iso-frequency “Acute_Post blast” data with ** corresponding to p<0.01 and *** corresponding to p<0.001. No significant changes were observed in control rats (Control_Pre-sham blast and Control_Post-sham blast).

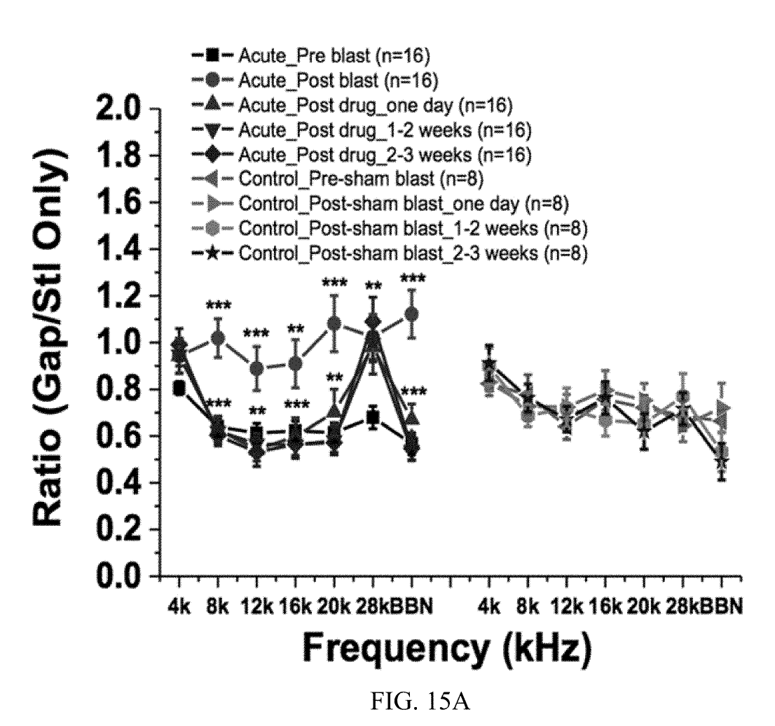

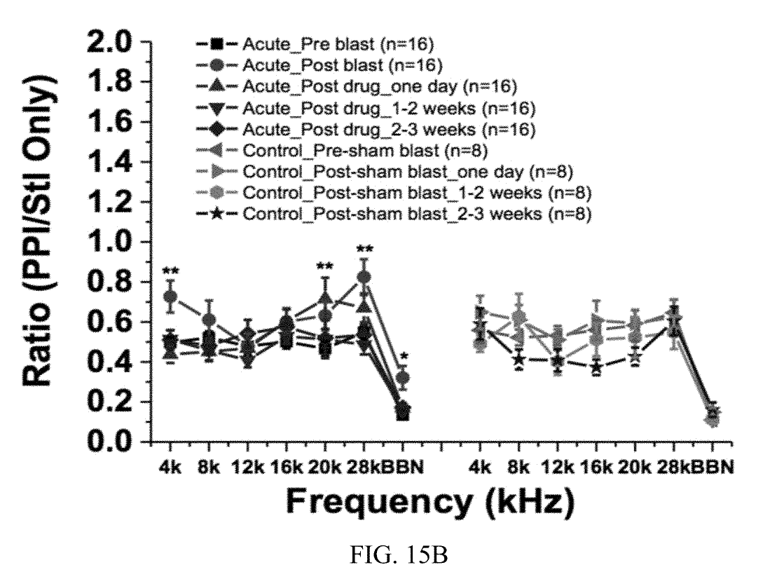

FIG. 15A depicts gap ratio values (gap/startle-only response, “Gap/Stl Only”) measured from 3,6′-dithiothalidomide acute tinnitus rats and control rats. Blast rats showed significant deficits in the gap test at 7 days after blast exposure (Acute_Post blast) and recovered significantly after 5 days of treatment with 3,6′-dithiothalidomide (Acute_Post drug). Asterisks over the “Acute_Post blast” data points indicate differences relative to iso-frequency “Acute_Pre blast” data with ** corresponding to p<0.01 and *** corresponding to p<0.001. Asterisks over the “Acute_Post drug_one day” data points indicate differences relative to iso-frequency “Acute_Post blast” data with ** corresponding to p<0.01 and *** corresponding to p<0.001. No significant changes were observed in control rats (Control_Pre-sham blast and Control_Post-sham blast).

FIG. 15B depicts PPI ratio values (PPI/startle-only response, “PPI Stl Only”) measured from 3,6′-dithiothalidomide-acute tinnitus rats and control rats. Blast rats showed significant deficits in the gap test at 7 days after blast exposure (Acute_Post blast) and recovered significantly after 5 days of treatment with 3,6′-dithiothalidomide (Acute_Post drug). Asterisks over the “Acute_Post blast” data points indicate differences relative to iso-frequency “Acute_Pre blast” data with * corresponding to p<0.05 and ** corresponding to p<0.01. No significant changes were observed in control rats (Control_Pre-sham blast and Control_Post-sham blast).

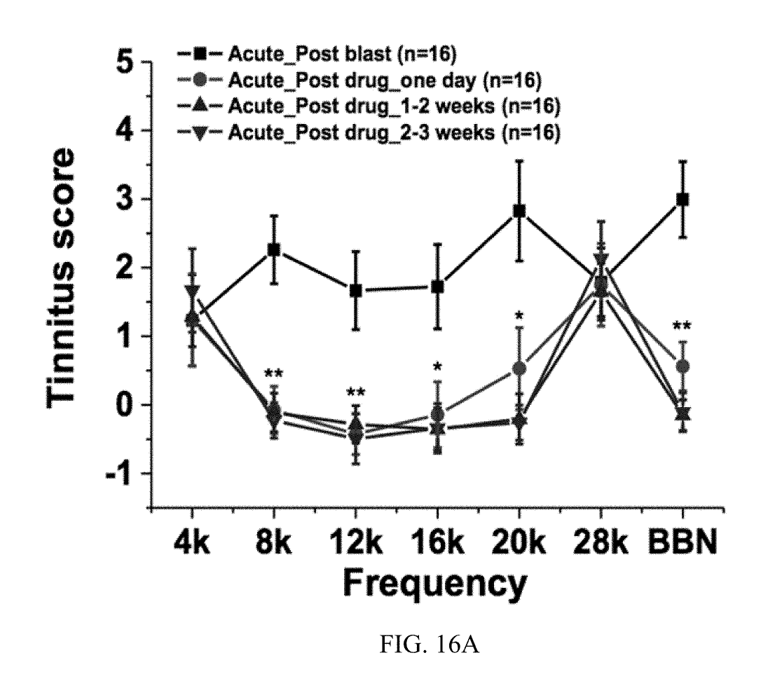

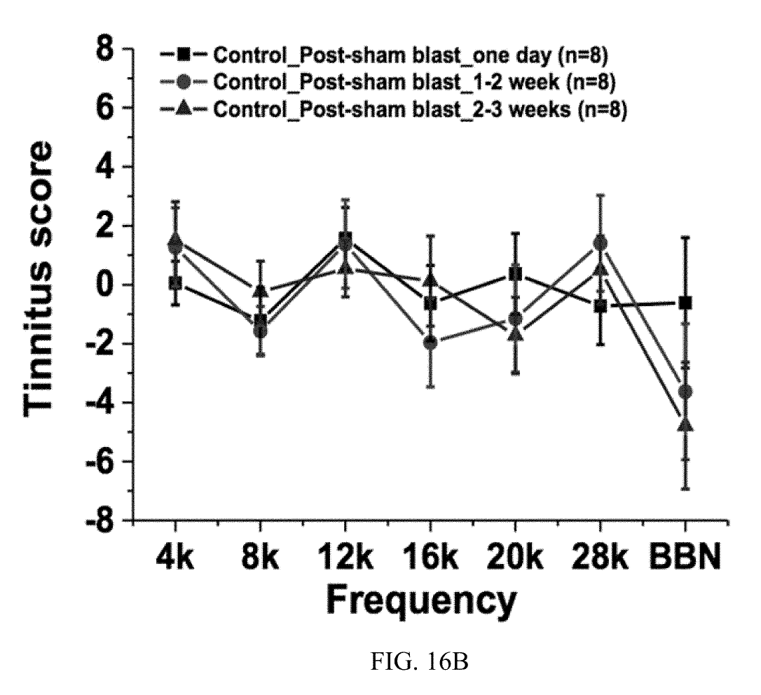

FIG. 16A depicts tinnitus scores measured from acute tinnitus rats and control rats. Blast rats displayed tinnitus 7 days after blast exposure (Acute_Post blast) and recovered significantly after 5 days of treatment with 3,6′-dithiothalidomide (Acute_Post drug). FIG. 16B demonstrates that no significant changes were observed in control rats (Control_Post-sham blast).

### Example 12: 3,6′-Dithiothalidomide Attenuates Blast Exposure-Induced GABA Release

Two groups of 7 rats each were exposed to blast shockwaves as described above, and an additional group of 7 rats was sham-exposed. One of the exposed groups was administered 3,6′-dithiothalidomide for five days starting on the day of the exposure, and the other exposed group was administered vehicle. Brain slides were prepared from the AC of the rats 10-20 days after the blast exposure. Pyramidal neurons were recorded using the patch clamp method. Miniature synaptic currents were recorded to assess the functional states of the excitatory and inhibitory synaptic transmission. Neurons were filled with biocytin, which was stained with immunocytochemistry to reveal the morphology of the recorded neurons.

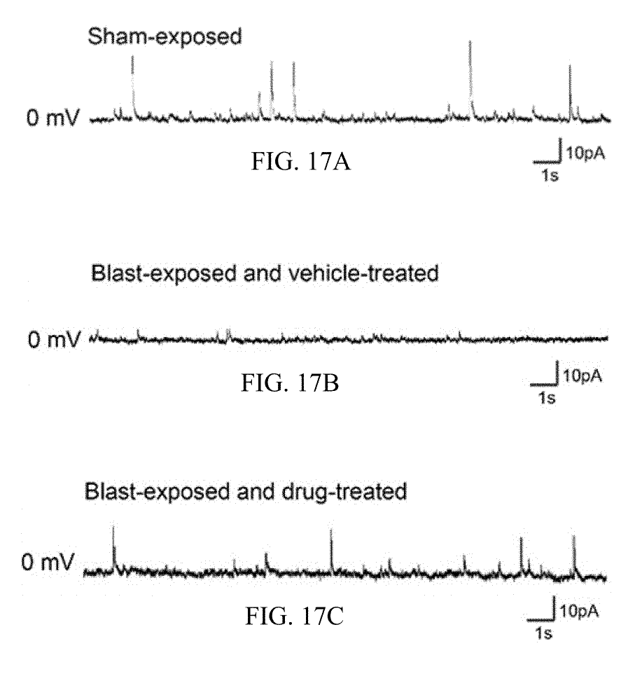

FIGS. 17A-17C show recording traces of auditory cortical neurons displaying miniature inhibitory postsynaptic currents (mIPSCs). The blast-exposed, vehicle-treated rats displayed reduced mIPSCs (FIG. 17B) relative to sham-exposed rats (FIG. 17A). The blast-exposed, 3,6′-dithiothalidomide-treated rats displayed less reduction in mIPSCs (FIG. 17C) relative to blast-exposed, vehicle-treated rats (FIG. 17B).

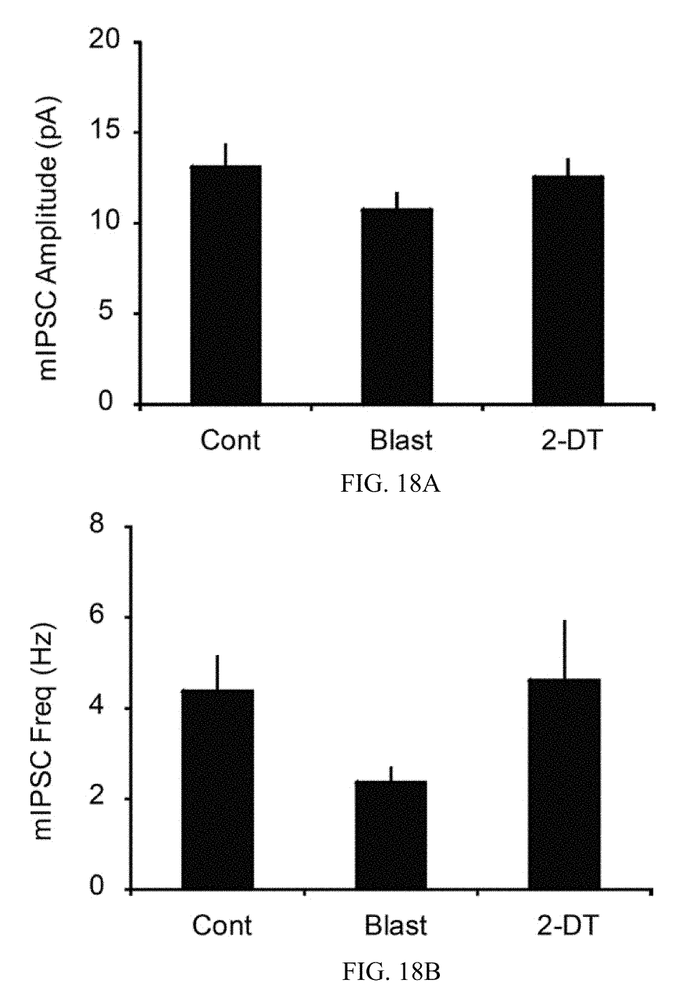

FIGS. 18A-18B show that the administration of 3,6′-dithiothalidomide (56 mg/kg, b.w., i.p.) rescues the blast-induced reduction of inhibitory synaptic transmission. FIG. 18A shows that blast-exposure did not significantly change mIPSC amplitudes in vehicle-treated rats (“Blast”) or 3,6′-dithiothalidomide-treated rats (2-DT) relative to sham-blast controls (“Cont”). The frequency of mIPSCs, however, was significantly reduced by blast exposure (FIG. 18B), indicative of a reduced probability of GABA release. Daily injection of 3,6′-dithiothalidomide completely abolished the blast-induced reduction (FIG. 18B).

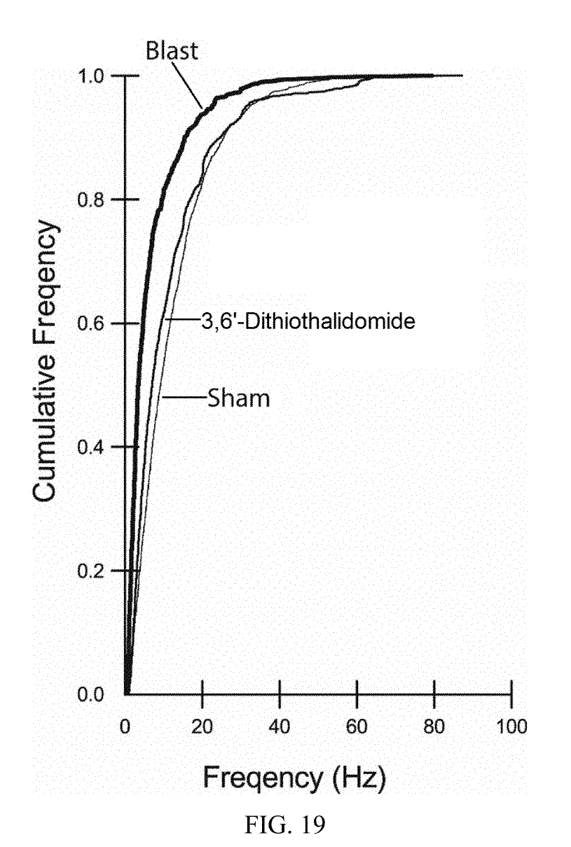

FIG. 19 shows the cumulative distribution of mIPSC frequency in sham-blast control rats (“Control”), vehicle-treated rats (“Blast”), and 3,6′-dithiothalidomide-treated rats. Blast exposure resulted in a shift to lower mIPSC frequencies relative to sham-blast controls (Kolmogorov-Smirnov Test, p<0.001), and the administration of 3,6′-dithiothalidomide (56 mg/kg, b.w., i.p.) reversed this effect (p<0.001).

### Example 13: 3,6′-Dithiothalidomide Abolishes Neural Correlates of Tinnitus in the DCN and Reduces them in the AC

Rats were exposed or sham-exposed to a blast shockwave. To determine whether TNF-α is involved in blast-induced tinnitus, the rats were injected once daily for 5 days with 3,6′-dithiothalidomide starting 7 days after blast exposure. Behavioral testing and ABR recordings started 1 day after drug injection and lasted for three weeks.

Neurophysiological recordings of the left DCN, right IC and right AC were performed 3 weeks after drug or vehicle injection. Each rat was anesthetized with a mixture of air (1 liter/min) and isoflurane (5% v/v) and then secured in a stereotaxic frame with hollow ear bars (model 1530; David Kopf Instruments, Tujunga, Calif.). A mixture of air (1 liter/min) and isoflurane (1.75-2.5% v/v) was used to maintain anesthesia during surgery and recordings. In order to maintain the rat's body temperature at 37° C., a thermostat-controlled blanket (Harvard Apparatus, Holliston, Mass.) was used during the procedure. For electrophysiological recording, electrode arrays were inserted into the DCN, IC, and AC. Prior to insertion, the electrode probe was dipped into a 3% DiI solution (1,10-dioctadecyl-3,3,30,30-tetramethylindocarbocyanine perchlorate; Invitrogen, Carlsbad, Calif.) prepared with dimethylformamide to label the electrode insertion tracks. By using a micromanipulator (model 1460-61; David Kopf Instruments), an eight-shank, 32-channel electrode probe (NeuroNexus Technologies, Ann Arbor, Mich.) was inserted into the DCN and the probe was inserted into a depth of 150-200 mm below the DCN surface, corresponding to the fusiform cell layer. For the IC, a two-shank 32-Channel (NeuroNexus) probe was inserted 20° off of the sagittal plane and 30° off of the horizontal plane through the occipital cortex into the IC. To implant in the AC, a 32 (4×8) microwire array was implanted 2.7 to 5.8 mm posterior to the bregma, and ˜0.8-1.0 mm from the cortical surface. After probe placement, the brain was covered with agarose to avoid tissue swelling and drying. The probe connector was connected to a real-time signal processing system (RZ2, TDT) with a 25 kHz sampling rate and a 100-3000 Hz bandpass filter. Spontaneous activity, frequency tuning curves (FTCs), and acoustic stimulation with tone and noise bursts (BBN) was recorded. Spontaneous single- and multi-unit spikes were recorded twice; one 5 min prior to and one 5 min after frequency tuning curve (FTC) construction. Each spontaneous recording period lasted 5 min. FTCs were obtained to determine the frequency representation of each implanted electrode in the DCN, IC and AC using tone sweeps (50 ms in duration, 2-44 kHz, incremental steps of 2 dB, sound level range of 0-85 dB SPL).

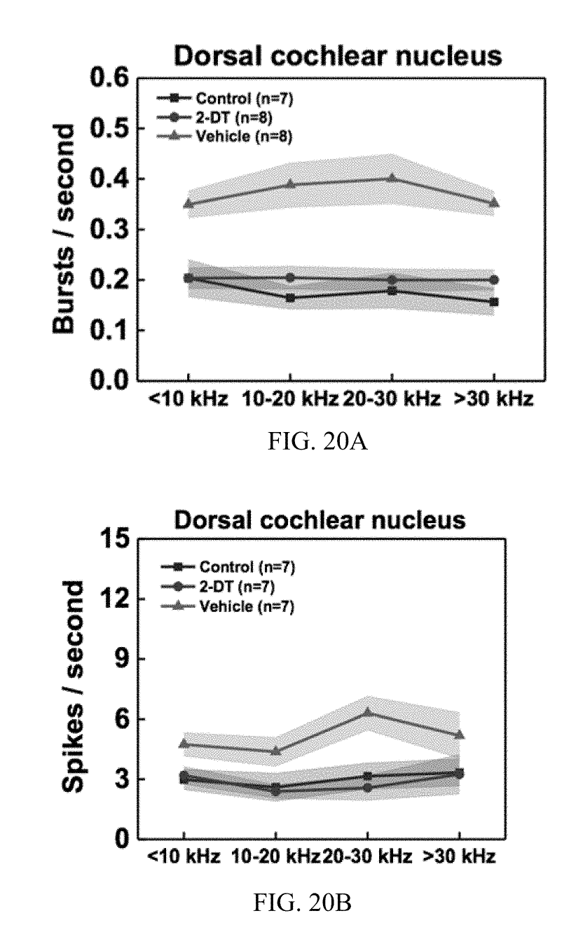

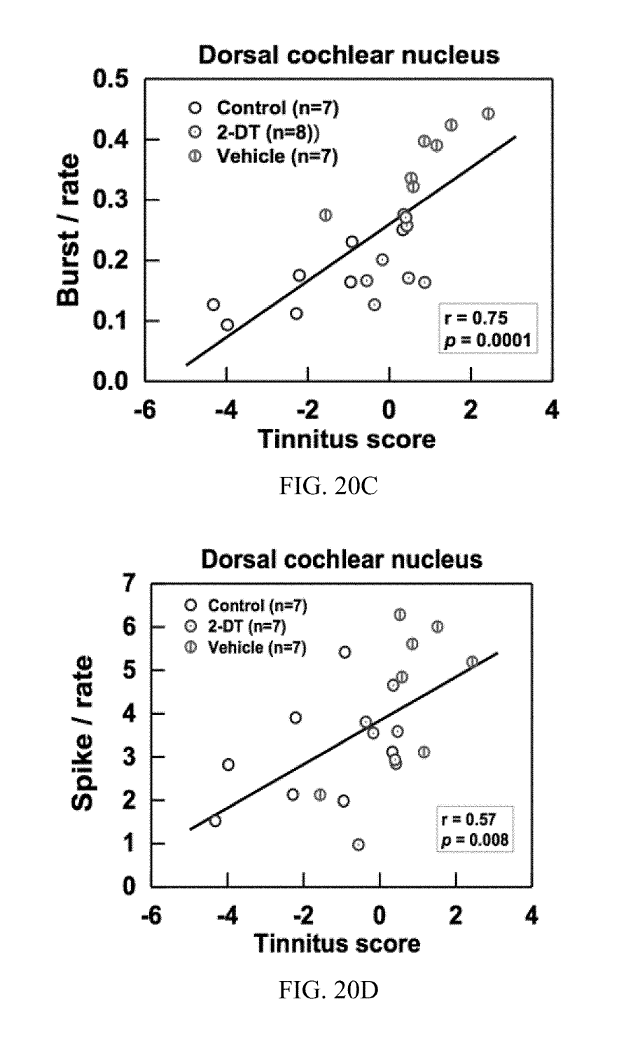

FIGS. 20A-20C depict comparisons of neural activity in the DCN of blast-exposed, 3,6′-dithiothalidomide-treated rats (2-DT), blast-exposed, vehicle-treated rats (Vehicle), and sham-blast control rats (Control).

FIG. 20A shows that the spontaneous bursting rate in the DCN increased following blast exposure (FIG. 20A, Vehicle and Control). Treatment with 3,6′-dithiothalidomide (56 mg/kg, b.w., i.p.) reduced bursting rates in blast-exposed animals to the control level (FIG. 20A, 2-DT and Control). FIG. 20B shows that the spontaneous firing rate in the DCN increased following blast exposure (FIG. 20B, Vehicle and Control). Treatment with 3,6′-dithiothalidomide (56 mg/kg, b.w., i.p.) reduced firing rates in blast-exposed animals to the control level (FIG. 20B, 2-DT and Control). The shaded areas in FIGS. 20A and 20B represent the 95% confidence level.

FIG. 20C and FIG. 20D show Pearson correlation results indicating that the spontaneous burst rate and spontaneous firing rate, respectively, were significantly correlated with the tinnitus score measured in each individual animal. These results indicate that spontaneous burst and firing rates in DCN are neural correlates of tinnitus and that 3,6′-dithiothalidomide treatment after blast exposure abolishes these neural correlates of tinnitus.

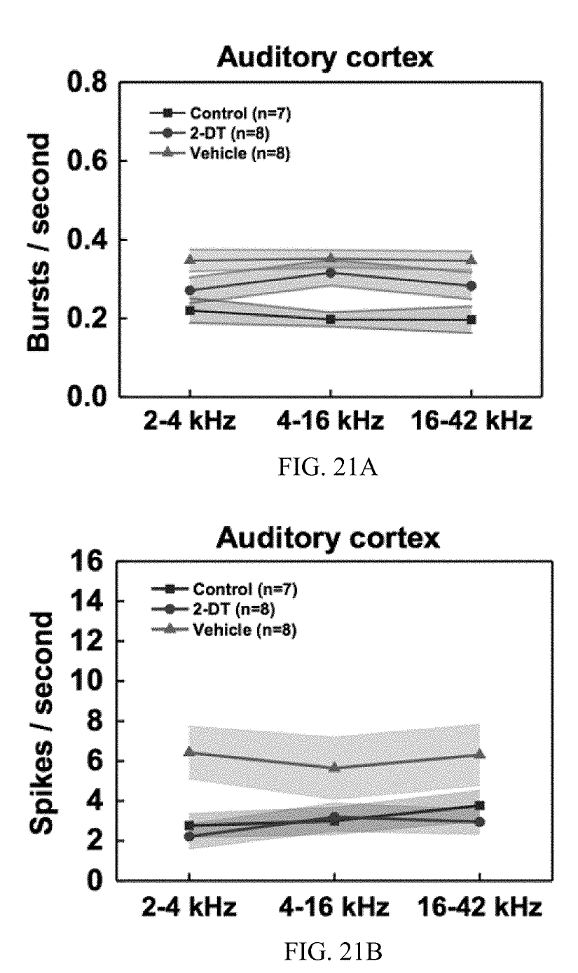

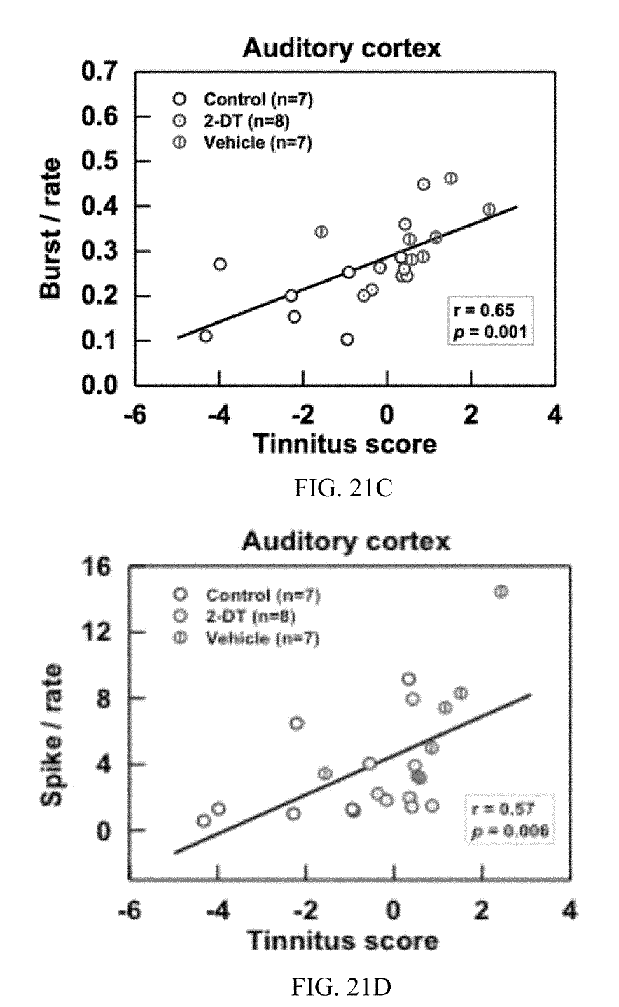

FIG. 21A shows that the spontaneous bursting rate in the AC increased following blast exposure (FIG. 21A, Vehicle and Control). Treatment with 3,6′-dithiothalidomide (56 mg/kg, b.w., i.p.) reduced bursting rates in blast-exposed animals relative to vehicle (FIG. 21A, 2-DT and Vehicle). FIG. 21B shows that the spontaneous firing rate in the AC increased following blast exposure (FIG. 21B, Vehicle and Control). Treatment with 3,6′-dithiothalidomide (56 mg/kg, b.w., i.p.) reduced firing rates in blast-exposed animals to the control level (FIG. 21B, 2-DT and Control). The shaded areas in FIGS. 21A and 21B represent the 95% confidence level.

FIG. 21C and FIG. 21D show Pearson correlation results indicating that the spontaneous burst rate and spontaneous firing rate, respectively, were significantly correlated with the tinnitus score measured in each individual animal. These results indicate that spontaneous burst and firing rates in AC are neural correlates of tinnitus and that 3,6′-dithiothalidomide treatment after blast exposure significantly reduces or abolishes these neural correlates of tinnitus.

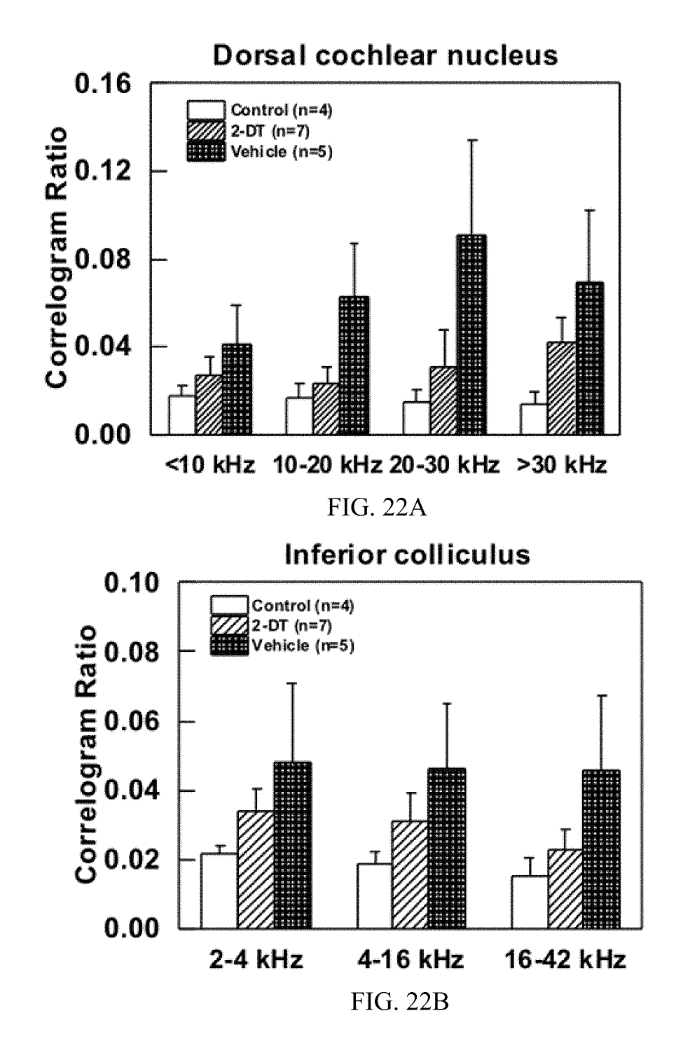

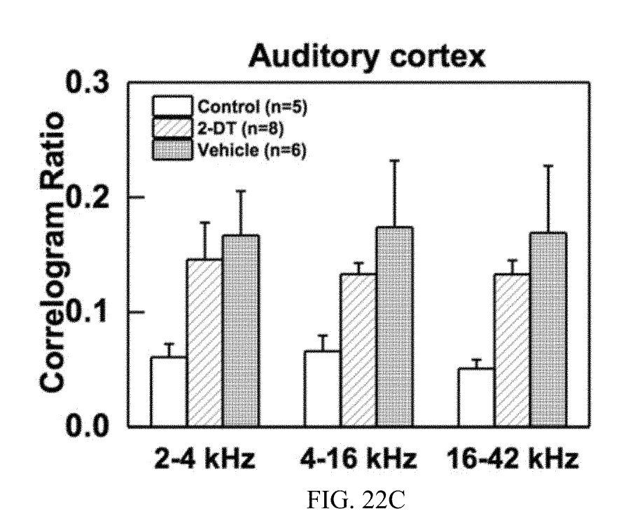

FIGS. 22A, 22B, and 22C show comparisons of neural synchrony as assessed by correlogram ratios at various frequency ranges in DCN, IC, and AC, respectively. Compared to sham blast-exposed controls (Control), blast-exposed rats displayed significantly higher cross correlation between recorded neurons (Vehicle), indicating increased neuronal firing synchrony, which has also been considered a neural correlate of tinnitus. The increase in neuronal firing synchrony was observed in all three brain regions investigated: DCN, IC, and AC. Treatment with 3,6′-dithiothalidomide (56 mg/kg, b.w., i.p.) significantly reduced neuronal firing synchrony in blast-exposed animals (2-DT). Error bars in FIGS. 22A-22C depict standard deviation.

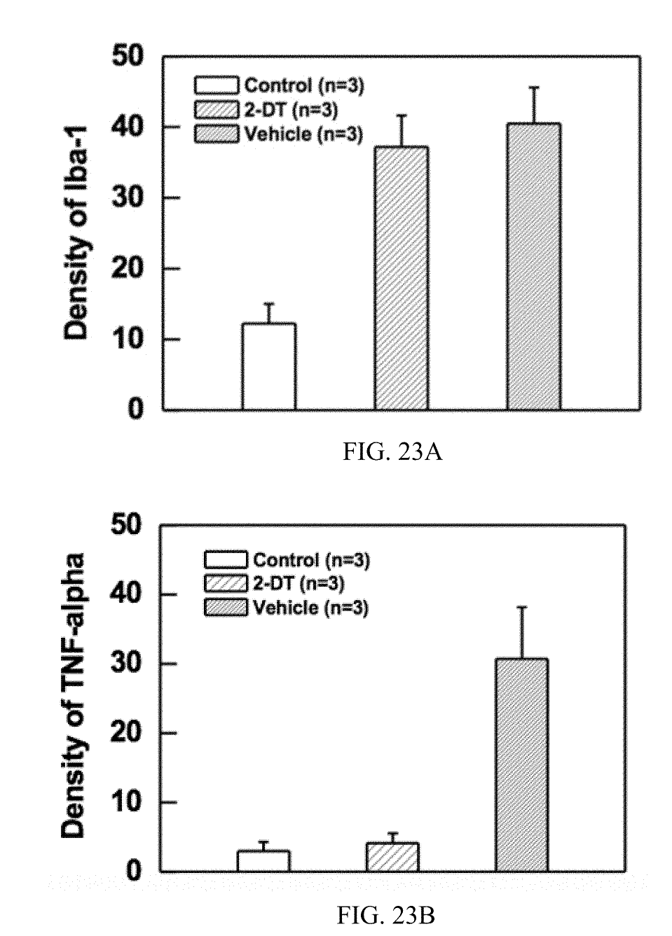

FIGS. 23A and 23B depict densitometry measurements of ionized calcium-binding adapter molecule 1 (Iba-1) and TNF-α expression, respectively, as observed by fluorescence microscopy of immunohistochemistry-stained microglia. FIG. 23A is a graph of densitometry measurements from fluorescence microscopy images demonstrating that blast-induced microglial activation (Vehicle) is not blocked by 3,6′-dithiothalidomide treatment (2-DT) as evidenced by Iba-I staining. FIG. 23B is a graph of densitometry measurements from fluorescence microscopy images demonstrating that blast-induced microglial TNF-α expression (Vehicle) is blocked by 3,6′-dithiothalidomide treatment (2-DT). Error bars in FIGS. 23A-23B depict standard deviation.

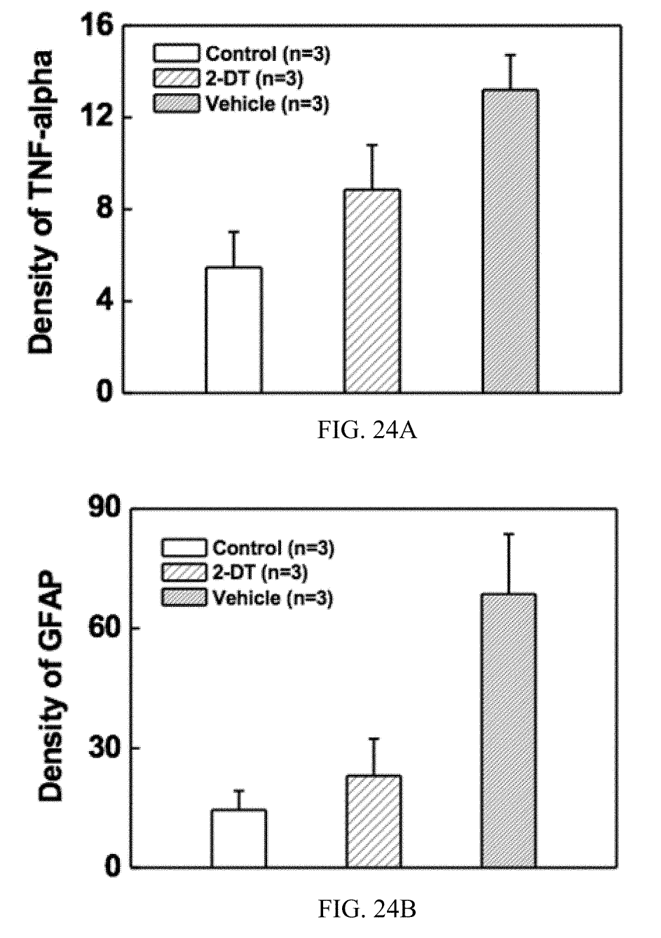

FIGS. 24A and 24B depict densitometry measurements of glial fibrillary acidic protein (GFAP) and TNF-α expression, respectively, as observed by fluorescence microscopy of immunohistochemistry-stained AC astrocytes. FIG. 24A is a graph of densitometry measurements from fluorescence microscopy images demonstrating that blast-induced astrocyte TNF-α expression (Vehicle) is partially blocked by 3,6′-dithiothalidomide treatment (2-DT). The decrease in astrocyte TNF-α expression was relatively smaller than the decrease in microglial TNF-α expression (compare FIG. 24A with FIG. 23B). FIG. 24B is a graph of densitometry measurements from fluorescence microscopy images demonstrating that blast-induced astrocyte GFAP expression (Vehicle) is blocked by 3,6′-dithiothalidomide treatment (2-DT). Error bars in FIGS. 24A-24B depict standard deviation.

### Example 14: Administration of 3,6′-Dithiothalidomide Prevents Noise-Induced Tinnitus

A group of C75BL6 mice underwent 4 days of gap detection testing. On the fifth day, the mice were exposed to 123-dB noise in their left ears for two hours, which was followed immediately by the administration of 3,6′-dithiothalidomide at a dose of 28 mg/kg. The drug was administered daily for five days. On day 10 post noise exposure, gap detection tests were resumed to test the animals' performance. Impaired gap detection compared to prenoise exposure performance is behavioral evidence of tinnitus.

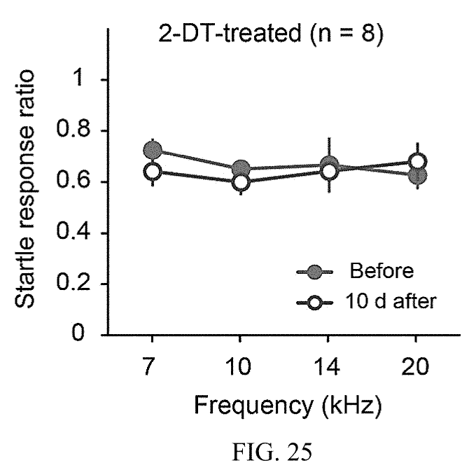

Gap detection performance was not significantly altered in animals that underwent noise-exposure and 3,6′-dithiothalidomide administration. FIG. 25 shows that the administration of 3,6′-dithiothalidomide (2-DT) at a dose of 28 mg/kg prevents noise-induced tinnitus in mice. This result suggest that 3,6′-dithiothalidomide prevents behavioral evidence of tinnitus in noise-exposed animals.

### Example 15: Administration of 3,6′-Dithiothalidomide Prevents the Noise-Induced Reduction of Cortical Inhibition

Three groups of C75BL6 mice were used. Two were exposed to 123-dB noise in the left ear for two hours. Animals in one of the two exposed group were administered 3,6′-dithiothalidomide. The drug was administered daily for five days. The remaining third group underwent the same procedure but without noise exposure or drug administration. On day 10 post-noise exposure, animals were euthanized and brain slices were prepared. Pyramidal neurons in the brain slices were recorded as described above. Miniature inhibitory and excitatory synaptic transmissions (mIPSC and mEPSC) were examined.

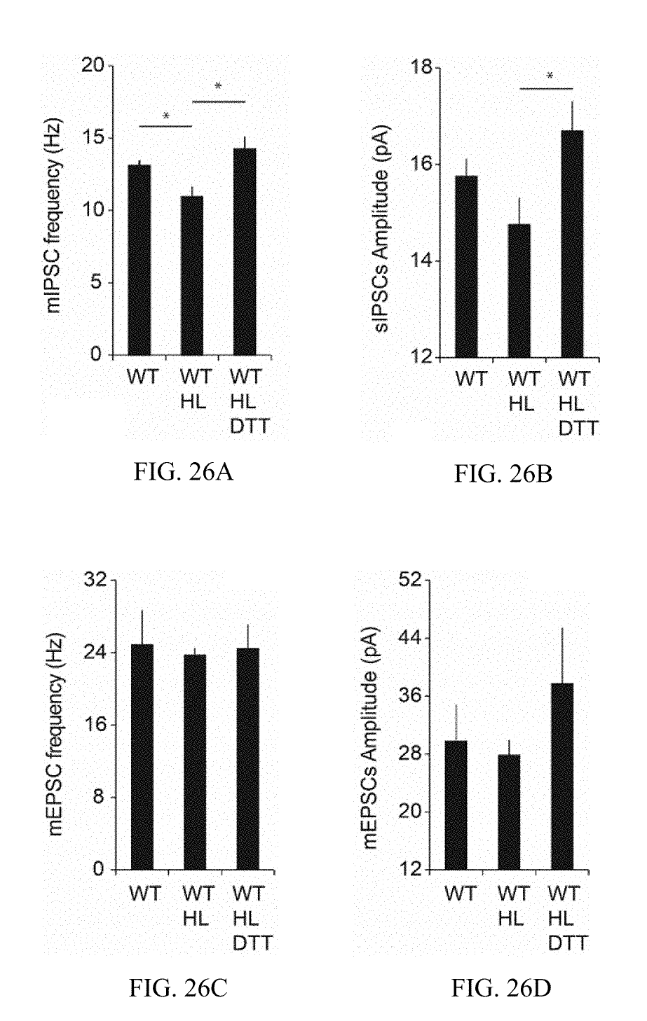

Noise exposure resulted in a significant reduction of the frequency of mIPSCs (FIG. 26A). This reduction was completely reversed by the administration of 3,6′-dithiothalidomide at a dose of 28 mg/kg (FIG. 26A). Other measurements were not altered by noise exposure.

FIG. 26A shows that exposure to 123-dB noise for two hours (“WT HL”) significantly reduces mIPSC frequency relative to control mice (“WT”), but the administration of 3,6′-dithiothalidomide at 28 mg/kg rescues the reduction in mIPSC frequency in noise-exposed mice (“WT HL DTT”).

FIG. 26B shows that exposure to 123-dB noise for two hours (“WT HL”) reduces mIPSC amplitude relative to control mice (“WT”), but the administration of 3,6′-dithiothalidomide at 28 mg/kg rescues the reduction in mIPSC amplitude in noise-exposed mice (“WT HL DTT”).

FIG. 26C shows that exposure to 123-dB noise for two hours has no effect on the mEPSC frequency of vehicle- or 3,6′-dithiothalidomide-treated mice (“WT HL” and “WT HL DTT,” respectively) relative to control mice (“WT”).

FIG. 26D shows that exposure to 123-dB noise for two hours has no significant effect on the mEPSC amplitude of vehicle- or 3,6′-dithiothalidomide-treated mice (“WT HL” and “WT HL DTT,” respectively) relative to control mice (“WT”).

### Example 16: Administration of Etanercept Prevents Blast-Induced Tinnitus

The experiments in Examples 10 and 11 were repeated with 5 mg/kg body weight i.p. etanercept as the TNF-α inhibitor instead of 3,6′-dithiothalidomide. Rats were blast-exposed as described in example 10, and injected with 5 mg/kg body weight i.p. etanercept or saline vehicle each day for 5 days starting 1 day after blast-exposure.

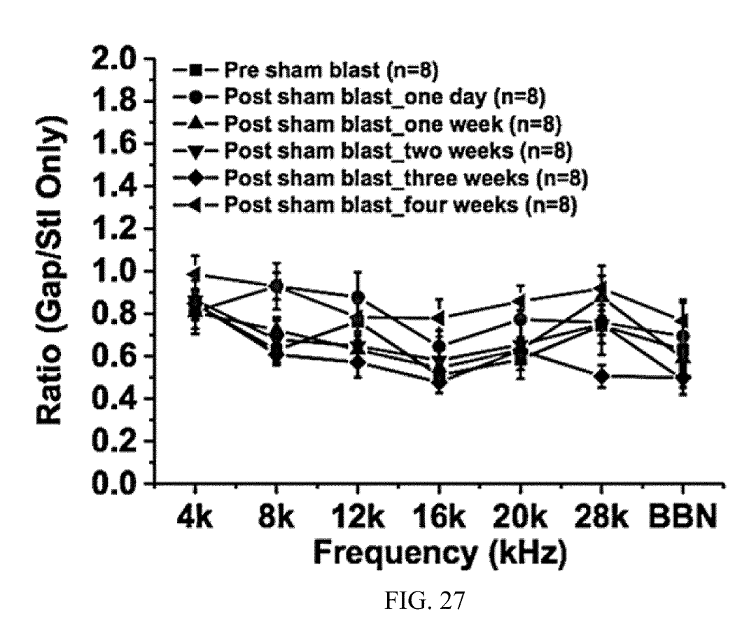

FIG. 27 depicts gap ratio values (gap/startle-only response, “Gap/SU Only”) measured in control rats. No significant changes were observed.

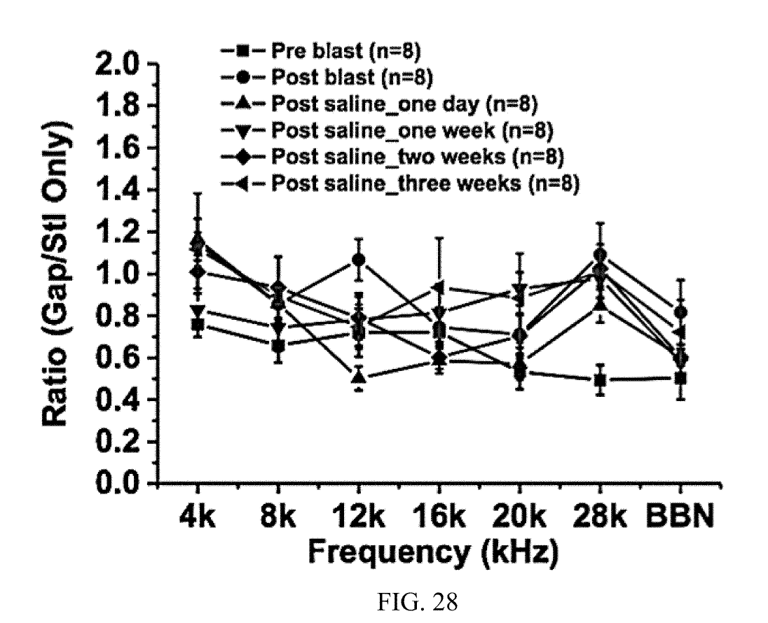

FIG. 28 depicts gap ratio values (gap/startle-only response, “Gap/Stl Only”) measured in vehicle-treated, blast-exposed rats. No therapeutic effects on tinnitus were observed. 28 kHz measurements, which correspond to the most common frequency of blast-induced tinnitus, displayed a marked deficit at all post-blast timepoints. Fluctuations in gap data were observed at low frequencies.

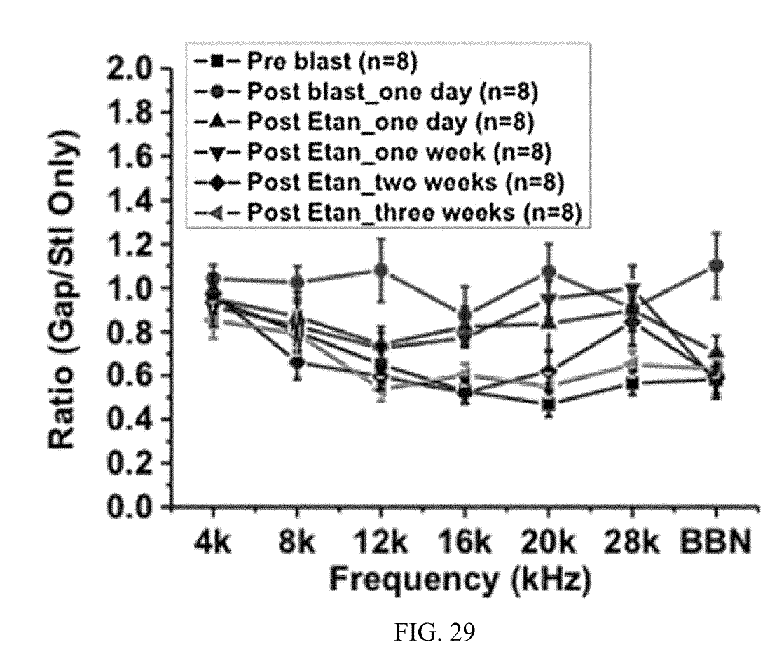

FIG. 29 depicts gap ratio values (gap/startle-only response, “Gap/Stl Only”) measured in pre-blast rats (Pre blast), blast-exposed rats one day after blast exposure (Post blast_one day), and etanercept-treated, blast-exposed rats (Post Etan). A significant therapeutic effect on tinnitus was observed at low frequencies and at 28 kHz at the two-week (Post Etan_two weeks) and three-week (post Etan_three weeks) timepoints. The therapeutic effect was less pronounced at the one-day (Post Etan_one day) and one-week (Post Etan_one week) timepoints, which suggests that etanercept has a delayed therapeutic effect relative to 3,6′-dithiothalidomide (compare FIG. 29 with FIG. 15A).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method of treating a hearing disorder associated with maladaptive neuroplasticity, reduction of inhibition, shift of excitation-to-inhibition balance, changes in central gain, and/or changes in neural sensitivity in a subject, the method comprising administering to the subject a tumor necrosis factor alpha (TNF-α) inhibitory agent in an amount effective to treat the subject for the hearing condition associated with maladaptive neuroplasticity, reduction of inhibition, shift of excitation-to-inhibition balance, changes in central gain, and/or changes in neural sensitivity.

2. The method according to claim 1, wherein the TNF-α inhibitory agent prevents TNF-α signaling or expression and is selected from the group consisting of a small molecule, a polypeptide, and a nucleic acid.

3. The method according to claim 1, wherein the TNF-α inhibitory agent directly binds TNF-α.

4. The method according to claim 1, wherein the TNF-α inhibitory agent directly binds a receptor for TNF-α.

5. The method according to claim 1, wherein the TNF-α inhibitory agent inhibits the expression of TNF-α mRNA.

6. The method according to claim 1, wherein the TNF-α inhibitory agent inhibits the translation of TNF-α protein.

7. The method according to claim 1, wherein the TNF-α inhibitory agent inhibits the release of TNF-α from cells.

8. The method according to claim 1, wherein the TNF-α inhibitory agent inhibits downstream signaling from a receptor for TNF-α.

9. The method according to claim 1, wherein the TNF-α inhibitory agent is an immune-modulatory drug.

10. The method according to claim 9, wherein the immune-modulatory drug is thalidomide, 3,6′-dithiothalidomide, or an analog of thalidomide or 3,6′-dithiothalidomide.

11. The method according to claim 1, further comprising administering to the subject a second agent selected from the group consisting of an ion channel inhibitor or enhancer, an enhancer of GABA signaling, an enhancer of glycine synapses, and an inhibitor of glutamate synapses.

12. The method according to claim 1, wherein the hearing disorder associated with maladaptive neuroplasticity, reduction of inhibition, shift of excitation-to-inhibition balance, changes in central gain, and/or changes in neural sensitivity to be treated in the subject is a result of one or more of: injury, an ototoxic drug or chemical agent, cochlear surgical insertion, aging, a genetic factor, infection, and autoimmune disease.

13. The method according to claim 1, wherein the hearing disorder associated with maladaptive neuroplasticity, reduction of inhibition, shift of excitation-to-inhibition balance, changes in central gain, and/or changes in neural sensitivity is a primary condition.

14. The method according to claim 1, wherein the hearing disorder is a secondary condition in the subject.

15. The method according to claim 1, wherein the hearing disorder associated with maladaptive neuroplasticity, reduction of inhibition, shift of excitation-to-inhibition balance, changes in central gain, and/or changes in neural sensitivity is a result of a traumatic brain injury (TBI).

16. The method according to claim 1, wherein the hearing disorder associated with maladaptive neuroplasticity, reduction of inhibition, shift of excitation-to-inhibition balance, changes in central gain, and/or changes in neural sensitivity to be treated in the subject is a result of noise and/or blast exposure.

17. The method of claim 16, wherein the TNF-α inhibitory agent is administered to the subject after the subject has been exposed to the noise or blast.

18. The method of claim 17, wherein the TNF-α inhibitory agent is administered to the subject at least once a day for at least two consecutive days starting within 24 hours to 10 days after the subject has been exposed to the noise or blast.

19. The method according to claim 1, wherein the hearing disorder associated with maladaptive neuroplasticity, reduction of inhibition, shift of excitation-to-inhibition balance, changes in central gain, and/or changes in neural sensitivity to be treated in the subject is a result of exposure to an ototoxic drug selected from the group consisting of: aminoglycoside, gentamycin, cisplatin, carboplatin, salicylate, quinine and combinations of any two or more thereof.

20. The method according to claim 1, wherein the hearing disorder associated with maladaptive neuroplasticity, reduction of inhibition, shift of excitation-to-inhibition balance, changes in central gain, and/or changes in neural sensitivity is selected from tinnitus, hyperacusis, and auditory processing deficit.

21. The method according to claim 1, wherein the subject does not present with hearing loss.

22. A method of treating a hearing disorder in a subject, the method comprising disrupting one or more alleles of a TNF-α signaling pathway gene in a cell of the subject in a manner effective to treat the subject for the hearing disorder.

23. The method according to claim 22, wherein the TNF-α signaling pathway gene is selected from the group consisting of TNF-α, a TNF-α receptor, and a downstream gene of the TNF-α signaling pathway.

24. The method according to claim 22, wherein the cell is a cell of the inner ear or the brain.

25. The method according to claim 1, wherein the TNF-α inhibitory agent is selected from the group consisting of: 3,6′-dithiothalidomide, etanercept, adalimumab, infliximab, SSR150106 and a combination of any two or more thereof.
